(19) United States
(12) Patent Application Publication
(10) Pub. No.: US 2025/0002586 A1
(43) Pub. Date: Jan. 2, 2025

(54) DUAL MHC-TARGETING T CELL ENGAGER

(71) Applicant: CDR-LIFE AG, Horgen (CH)

(72) Inventors: Leonardo BORRAS, Birmensdorf (CH); Stephanie JUNGMICHEL, Zürich (CH); Hannes MERTEN, Zürich (CH); Philipp Robert RICHLE, Zürich (CH); Fabian Bert SCHEIFELE, Mägenwil-Aargau (CH); Anna Maria SOBIERAJ, Zürich (CH)

(21) Appl. No.: 18/736,330

(22) Filed: Jun. 6, 2024

Related U.S. Application Data
(63) Continuation of application No. PCT/EP2022/085689, filed on Dec. 13, 2022.
(60) Provisional application No. 63/328,417, filed on Apr. 7, 2022.
(60) Provisional application No. 63/317,256, filed on Mar. 7, 2022.
(60) Provisional application No. 63/289,380, filed on Dec. 14, 2021.

(51) Int. Cl. C07K 16/28

(52) U.S. Cl. CPC C07K 16/2833; C07K 16/2809; C07K 2317/31; C07K 2317/35; C07K 2317/522; C07K 2317/55; C07K 2317/569; C07K 2317/622; C07K 2317/92

(57) ABSTRACT

Described herein are antigen binding proteins comprising a Fab domain which specifically binds to a cell surface protein of an immune cell, a first pMHC binding domain, and a second pMHC binding domain. Methods of treating cancer or a viral infection with the same are also described.

### RELATED APPLICATIONS

This application is a continuation of International Patent Application No. PCT/EP2022/085689, filed Dec. 13, 2022, which claims the benefit of U.S. Provisional Application Ser. No. 63/289,380, filed Dec. 14, 2021, U.S. Provisional Application Ser. No. 63/317,256, filed Mar. 7, 2022, and U.S. Provisional Application Ser. No. 63/328,417, filed Apr. 7, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jun. 6, 2024, is named 755089_CDR9-007PCCON_ST26.xml and is 111,572 bytes in size.

### FIELD OF THE DISCLOSURE

The present disclosure relates to highly potent T-cell engager antibody formats that bind to tumor peptide-MHC (pMHC) complexes with high specificity and further comprise a CD3 targeting moiety in a Fab format. Such pMHC T-cell engagers rely on bivalent pMHC binding and a monovalent CD3 binding in different affinities for cytokine release tunning. The bivalent targeting of pMHCs on cancer cells provides efficient T-cell mediated cancer cell killing despite very low levels of pMHC on the cell surface.

### BACKGROUND

Peptide-MHC complexes (pMHCs) derived from intracellular tumor associated antigens (TAAs) represent a large repertoire of novel targets for immunotherapy. pMHCs are present on the surface of virtually all nucleated cells and are constantly surveilled by T-cells. Upon pMHC binding by T-cell receptors (TCRs), infected and/or malignantly transformed cells are recognized and eliminated. Thus, intracellular tumor associated proteins presented as peptides on MHC class I molecules are attractive targets for immunotherapeutic approaches with promising data already emerging from clinical trials. pMHCs have been traditionally targeted by TCR-engineered T cells or soluble recombinant T-cell receptors (TCRs) fused to an anti-CD3 fragment. However, naturally occurring cancer reactive TCRs typically exhibit binding affinities between 0.1-500 μM for their pMHC targets. Therefore, they need substantial engineering efforts to endow them with the necessary binding affinity and biophysical properties to be developed as drugs which may compromise the required specificity to the pMHC target. Conversely, developing high-affinity soluble antibody molecules with high specificity to pMHCs derived from the intracellular tumor associated antigens, addresses the challenging low affinity of TCRs which require significant affinity enhancements.

### SUMMARY

The present disclosure relates to antigen binding proteins comprising a Fab domain which specifically binds to a cell surface protein of an immune cell, the Fab domain comprising a heavy chain and a light chain; at least a first pMHC binding domain operably linked to the heavy chain, wherein the first pMHC binding domain binds to first target peptide-MHC (pMHC) complex; and c) at least a second pMHC binding domain operably linked to the light chain, wherein the second pMHC binding domain binds to a second pMHC complex. Bivalent targeting of pMHCs with the bispecific antigen binding proteins of the invention results in increased cancer cell killing compared to their monovalent bispecific counterparts, while the overall specificity against cells bearing the same HLA allele but not expressing the target protein is not substantially affected.

The antigen binding proteins of the invention lack an Fc domain. The antigen binding proteins of the disclosure therefore are not recognized through Fc-receptors on effector cells, such as the Fc-receptor FcγRIII on macrophages and activated neutrophils, or inhibiting receptors such as FcγRIIb, and on FcγRIIa complexes on non-cytotoxic cells such as platelets and B-cells. For bispecific T-cell engagers, Fc-mediated immune functions are unwanted to avoid antigen-independent cytokine release syndrome (CRS) due to crosslinking of CD3 and Fcγ receptors followed by nonspecific activation of immune cells. Rather, the Fab domain of the antigen binding protein serves as a specific heterodimerization scaffold to which the additional pMHC binding domains are linked. The natural and efficient heterodimerization properties of the heavy chain (Fd fragment) and light chain (L) of a Fab fragment makes the Fab fragment a useful scaffold. Additional binding domains may be in several different formats, including, but not limited to, another Fab domain, a scFv, or an sdAb. Moreover, in certain contexts, an Fc-containing antigen binding protein may be disadvantageous due to increased half-life. An extended half-life may lead to increased toxicity from, among other things, excess cytokine release from immune cells. The extended half-life may also promote T cell exhaustion. The antigen binding proteins of the disclosure lacking an Fc domain may possess reduced cytotoxicity in part due to a reduce half-life relative to an Fc-containing antigen binding protein.

In one aspect, the disclosure provides an antigen binding protein comprising: a) a single Fab domain which specifically binds to a cell surface protein of an immune cell, the Fab domain comprising a heavy chain and a light chain; b) at least a first pMHC binding domain operably linked to the heavy chain, wherein the first pMHC binding domain binds to first target peptide-MHC (pMHC) complex; and c) at least a second pMHC binding domain operably linked to the light chain, wherein the second pMHC binding domain binds to a second pMHC complex, wherein antigen binding protein does not comprise an Fc domain.

In certain embodiments, the Fab domain heavy chain comprises a CH1 domain and a VH domain, and at least 5 amino acids of an antibody hinge region. In certain embodiments thereof, the Fab domain heavy chain comprises at most 10 amino acids of an antibody hinge region at the C-terminus of the CH1 domain. In certain embodiments, the Fab domain heavy chain comprises 5-10 amino acids of an antibody hinge region at the C-terminus of the CH1 domain. In certain embodiments, said at least 5 amino acids or said at most 10 amino acids of an antibody hinge region comprise the sequence EPKSC (SEQ ID NO.: 87). Additionally, the at least 5 amino acids, respectively the at most 10 amino acids of an antibody hinge region, may be followed by a GGGGS (SEQ ID NO.: 88) linker.

In certain embodiments, the Fab domain light chain comprises a CL domain and a VL domain. The CL domain may be followed by a linker, such as GGGGS (SEQ ID NO.:88).

In certain embodiments, the first target pMHC complex and the second target pMHC complex are the same. In certain embodiments, the first target pMHC complex and the second target pMHC complex are different.

In certain embodiments, the first pMHC binding domain is operably linked to the C-terminus of the heavy chain or the N-terminus of the heavy chain. In certain embodiments, the second pMHC binding domain is operably linked to the C-terminus of the heavy chain or the N-terminus of the heavy chain.

In certain embodiments, the first pMHC binding domain is operably linked to the C-terminus of the light chain or the N-terminus of the light chain. In certain embodiments, the second pMHC binding domain is operably linked to the C-terminus of the light chain or the N-terminus of the light chain.

In certain embodiments, the pMHC binding domain is a scFv or an sdAb. As described elsewhere herein, the pMHC binding domain may also be any one of a scFab, a diabody or a Fab.

In certain embodiments, the antigen binding protein comprises: 1) a first pMHC binding scFv linked to the C-terminus of the Fab domain heavy chain and a second pMHC binding scFv linked to the C-terminus of the Fab domain light chain; 2) a first pMHC binding scFv linked to the N-terminus of the Fab domain heavy chain and a second pMHC binding scFv linked to the N-terminus of the Fab domain light chain; 3) a first pMHC binding scFv linked to the N-terminus of the Fab domain heavy chain and a second pMHC binding scFv linked to the C-terminus of the Fab domain light chain; 4) a first pMHC binding scFv linked to the C-terminus of the Fab domain heavy chain and a second pMHC binding scFv linked to the N-terminus of the Fab domain light chain; 5) a first pMHC binding sdAb linked to the N-terminus of the Fab domain heavy chain and a second pMHC binding sdAb linked to the N-terminus of the Fab domain light chain; 6) a first pMHC binding sdAb linked to the C-terminus of the Fab domain heavy chain and a second pMHC binding sdAb linked to the C-terminus of the Fab domain light chain; 7) a first pMHC binding sdAb linked to the N-terminus of the Fab domain heavy chain and a second pMHC binding sdAb linked to the C-terminus of the Fab domain light chain; or 8) a first pMHC binding sdAb linked to the C-terminus of the Fab domain heavy chain and a second pMHC binding sdAb linked to the N-terminus of the Fab domain light chain.

In certain embodiments, the first pMHC binding domain and/or the second pMHC binding domain comprise a variable heavy chain having a polar amino acid at position 11, 89 and/or 108, according to Kabat numbering.

In certain embodiments, the Fab domain comprises a variable heavy chain having a non-polar amino acid at position 11, 89 and/or 108, according to Kabat numbering.

In certain embodiments, the variable heavy chain comprises: leucine (L) or serine (S) at amino acid position 11, according to Kabat numbering; valine (V), serine (S), or threonine (T) at amino acid position 89, according to Kabat numbering; and/or leucine (L), serine (S), or threonine (T) amino acid position 108, according to Kabat numbering.

In certain embodiments, the polar amino acid is serine (S) and/or threonine (T).

In certain embodiments, the variable heavy chain comprises serine (S) at amino acid position 11, serine (S) or threonine (T) at amino acid position 89, and serine (S) or threonine (T) at amino acid position 108, according to Kabat numbering.

In certain embodiments, the variable heavy chain comprises serine (S) at amino acid position 11, serine (S) at amino acid position 89, and serine (S) at amino acid position 108, according to Kabat numbering.

In certain embodiments, the Fab domain comprises a variable heavy chain having a serine (S) at position 113 deleted, according to Kabat numbering.

In certain embodiments, the first pMHC binding domain and/or the second pMHC binding domain comprise a variable heavy chain having a serine (S) at position 113 deleted, according to Kabat numbering.

In certain embodiments, the Fab domain comprises a variable heavy chain having a serine (S) at position 112 deleted and a serine (S) at position 113 deleted, according to Kabat numbering.

In certain embodiments, the first pMHC binding domain and/or the second pMHC binding domain comprise a variable heavy chain having a serine (S) at position 112 deleted and a serine (S) at position 113 deleted, according to Kabat numbering.

In certain embodiments, the antigen binding protein comprises an S113A, S113G, or S113T substitution, according to Kabat numbering.

In certain embodiments, the antigen binding protein comprises an S113A, S113G, or S113T substitution, and wherein S112 is deleted, according to Kabat numbering.

In certain embodiments, the antigen binding protein comprises an S112A, S112G, or S112T substitution, according to Kabat numbering.

In certain embodiments, the antigen binding protein comprises an S112A, S112G, or S112T substitution, and wherein S113 is deleted, according to Kabat numbering.

In certain embodiments, the target pMHC binding domain specifically targets an MHC restricted peptide derived of a tumor antigen or a viral antigen.

In certain embodiments, the cell surface protein of an immune cell is selected from the group consisting of CD3, TCRα, TCRβ, CD16, NKG2D, CD89, CD64, and CD32a. In certain embodiments, the cell surface protein of an immune cell is CD3.

In certain embodiments, the immune cell is selected from the group consisting of a T cell, a B cell, a natural killer (NK) cell, a natural killer T (NKT) cell, a neutrophil cell, a monocyte, and a macrophage. In certain embodiments, the immune cell is a T cell.

In certain embodiments, the Fab domain specifically binds to CD3 with a binding affinity (K_{D}) between about 1 nM to about 50 nM, optionally between about 20 nM to 50 nM, as determined by SPR.

In certain embodiments, the Fab domain specifically binds to CD3 with a binding affinity (K_{D}) of about 1 nM, of about 10 nM, or of about 50 nM, as determined by SPR.

In certain embodiments, the first pMHC binding domain and/or the second pMHC binding domain bind the target pMHC complex with a binding affinity (K_{D}) of about 100 pM to about 5 nM. In certain embodiments, the first pMHC binding domain and/or the second pMHC binding domain bind the target pMHC complex with a binding affinity (K_{D}) of about 500 pM to about 5 nM, to about 10 nM, or to about 20 nM.

In certain embodiments, the antigen binding protein comprises a molecular weight of about 75 kDa to about 100 kDa, or of about 75 kDa to about 105 kDa or 110 kDa.

In certain embodiments, the antigen binding protein has increased serum half-life relative to an antigen binding protein with a molecular weight of less than about 60 kDa.

Thus, in one aspect, the disclosure provides an antigen binding protein comprising: a) a single Fab domain which specifically binds CD3 on a T cell, the Fab domain comprising a heavy chain and a light chain; b) at least a first pMHC binding domain operably linked to the C-terminus of the heavy chain, wherein the first pMHC binding domain binds to first target peptide-MHC (pMHC) complex; and c) at least a second pMHC binding domain operably linked to the C-terminus of the light chain, wherein the second pMHC binding domain binds to a second pMHC complex, wherein antigen binding protein does not comprise an Fc domain.

In one aspect, the disclosure provides a method of reducing nonspecific T cell activation of a T cell engaging multispecific antigen binding protein, wherein the multispecific antigen binding protein comprises a first binding domain specifically targeting CD3 and a second binding domain specifically targeting a tumor antigen, wherein the multispecific antigen binding protein comprises at least one variable heavy chain, the method comprising the step of: a) substituting a variable heavy chain amino acid at position 11, 89, and/or 108, according to Kabat numbering, with a polar amino acid.

In certain embodiments, the method further comprises the step of: b) deleting a serine (S) at position 113, according to Kabat numbering.

In certain embodiments, the polar amino acid of step a) is serine (S) and/or threonine (T).

In certain embodiments, the heavy chain amino acid is substituted with serine (S) at heavy chain amino acid position 11, serine (S) or threonine (T) at heavy chain amino acid position 89, and/or serine (S) or threonine (T) at heavy chain amino acid position 108, according to Kabat numbering.

In certain embodiments, the heavy chain amino acid is substituted with serine (S) at heavy chain amino acid position 11, serine (S) at heavy chain amino acid position 89, and serine (S) at heavy chain amino acid position 108, according to Kabat numbering.

In certain embodiments, step b) further comprises the step of deleting a serine (S) at position 112, according to Kabat numbering.

In certain embodiments, the method further comprises adding alanine (A), glycine (G) or threonine (T) at Kabat amino position 112 or 113.

In certain embodiments, the method comprises adding alanine (A) at Kabat amino position 112 or 113.

In certain embodiments, the substitutions and/or deletions are made in the heavy chain of the second binding domain.

In certain embodiments, the multispecific antigen binding protein is monovalent, bivalent or multivalent.

In certain embodiments, the antigen binding protein which may be used in such method is a Fab-sdAb, Fab-(sdAb)₂, a Fab-scFv or a Fab-(scFv)₂, F(ab′)₂fragment, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomAB, quadroma, CrossMab, DuoBody, triabody and tetrabody, or MATCH.

In certain embodiments, the second binding domain specifically targets a pMHC. In certain embodiments, the multispecific antigen binding protein further comprises a third binding domain specifically targeting a pMHC. In certain embodiments, the second binding domain and the third binding domain specifically target the same pMHC or different pMHC.

In certain embodiments, the antigen binding protein comprises one binding domain specifically targeting CD3 and one binding domain specifically targeting a pMHC.

In certain embodiments, the antigen binding protein comprises one binding domain specifically targeting CD3 and two binding domains specifically targeting a pMHC.

In certain embodiments, the two binding domains specifically targeting a pMHC are the same. In some embodiments, the two pMHC binding domains comprise the same set of six CDR sequences. In some embodiments, the two pMHC binding domains comprise the same VL and VH sequences.

Thus, in certain embodiments, the antigen binding protein is a Fab-(scFv)₂, wherein the Fab targets CD3 and one or both scFv target a tumor antigen, in particular a pMHC complex, such as a MAGE-A4 derived peptide presenting HLA as outlined below. The substitutions and/or deletions described herein are made in the heavy chain of the scFvs.

In certain embodiments, the pMHC binding domain specifically targets a MHC restricted peptide derived of a tumor antigen or a viral antigen.

In certain embodiments, the binding affinity (K_{D}) for CD3 is between about 1 nM to about 50 nM, optionally between about 20 nM to 50 nM, as determined by SPR. In certain embodiments, the binding affinity (K_{D}) for CD3 is of about 1 nM, of about 10 nM, or of about 50 nM, as determined by SPR. In certain embodiments, the binding affinity (K_{D}) for CD3 is of about 1 nM, of about 10 nM, or of about 50 nM, as determined by SPR.

In certain embodiments, the binding affinity (K_{D}) for the pMHC is of about 100 pM to about 20 nM, such as about 500 pM to about 10 nM or about 500 pM to about 5 nM or about 500 pM to about 2 nM.

In one aspect, the disclosure provides a multispecific antigen binding protein obtainable by the method described above.

In one aspect, the disclosure provides an antigen binding protein comprising at least one first binding domain specific for CD3 and at least one second binding domain specific for a tumor antigen, each binding domain comprising at least one variable heavy chain, wherein at least one variable heavy chain comprises a polar amino acid at position 11, 89 and/or 108, according to Kabat numbering.

In certain embodiments, the variable heavy chain is of said second binding domain.

In certain embodiments, the polar amino acid is serine (S) and/or threonine (T).

In certain embodiments, the variable heavy chain comprises serine (S) at heavy chain amino acid position 11, serine (S) or threonine (T) at heavy chain amino acid position 89, and serine (S) or threonine (T) at heavy chain amino acid position 108, according to Kabat numbering.

In certain embodiments, the variable heavy chain comprises serine (S) at heavy chain amino acid position 11, serine (S) at heavy chain amino acid position 89, and serine (S) at heavy chain amino acid position 108, according to Kabat numbering.

In certain embodiments, the variable heavy chain has a serine (S) at position 113 deleted, according to Kabat numbering.

In certain embodiments, the variable heavy chain has serine (S) at position 112 and 113 deleted, according to Kabat numbering.

In certain embodiments, the antigen binding protein comprises alanine (A), glycine (G) or threonine (T) at position 112, according to Kabat numbering, in particular alanine (A).

In certain embodiments, the antigen binding protein comprises alanine (A), glycine (G) or threonine (T) at position 112, according to Kabat numbering, in particular alanine (A).

In certain embodiments, the tumor antigen is a pMHC.

In certain embodiments, the pMHC binding domain specifically targets a MHC restricted peptide derived of a tumor antigen or a viral antigen.

In certain embodiments, the antigen binding protein has an affinity (K_{D}) for CD3 of about 1 nM to about 50 nM, preferably between about 20 nM to 50 nM, as determined by SPR. In certain embodiments, the antigen binding protein has an affinity (K_{D}) for CD3 of about 1 nM, of about 10 nM, or of about 50 nM, as determined by SPR.

In certain embodiments, the first binding domain specific for CD3 is a Fab fragment.

In certain embodiments, the antigen binding protein comprises two or more pMHC binding domains.

In certain embodiments, the pMHC binding domain is a scFv or an sdAb.

In certain embodiments, the antigen binding protein has an affinity (K_{D}) for the pMHC of about 100 pM to about 20 nM, such as about 500 pM to about 10 nM or about 500 pM to about 5 nM.

In certain embodiments, the antigen binding protein is a Fab-sdAb, Fab-(sdAb)₂, a Fab-scFv or a Fab-(scFv)₂, F(ab′)₂fragment, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomAB, quadroma, CrossMab, DuoBody, triabody and tetrabody, or MATCH.

In one aspect, the disclosure provides a method for killing a target cell comprising a major histocompatibility complex (MHC) presenting a neoantigen, the method comprising: a) contacting a plurality of cells comprising immune cells and the target cell with the antigen binding protein described above, wherein said antigen binding protein specifically binds to the pMHC on the surface of the target cell and to CD3 on the surface of the immune cells; b) forming a specific binding complex through the antigen binding protein interactions with the target cells and the immune cells, thereby activating the immune cells; and c) killing the target cell with the activated immune cells.

In one aspect, the disclosure provides a composition comprising an antigen binding protein described herein.

In one aspect, the disclosure provides a method of treating cancer comprising the step of administering the composition described above to a patient in need thereof.

In one aspect, the disclosure provides a nucleic acid encoding an antigen binding protein described herein.

An expression vector comprising the nucleic acid described above.

In one aspect, the disclosure provides a host cell population comprising the expression vector described above.

In one aspect, the disclosure provides a kit comprising an antigen binding protein described herein.

In one aspect, the disclosure provides a method of manufacturing an antigen binding protein as described herein, comprising the steps of: (i) cultivating the host cell described above under conditions allowing expression of the antigen binding protein described above; (ii) recovering the antigen binding protein or bispecific antigen binding protein; and optionally (iii) further purifying and/or modifying and/or formulating the antigen binding protein or bispecific antigen binding protein.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts a schematic of antigen binding protein formats used in Example 3 of the disclosure.

FIGS. 2A-2D depict in vitro cell killing in osteosarcoma cells incubated with monovalent pMHC-targeting T cell engagers in formats 1 and 2 (FIG. 2A), and 3 and 4 (FIG. 2B).

FIG. 2C depicts in vitro cell killing in osteosarcoma cells incubated with bivalent pMHC-targeting T cell engagers in formats 5 and 6. FIG. 2D depicts a direct comparison of in vitro cell killing of osteosarcoma cells mediated by monovalent and bivalent pMHC-targeting T cell engagers in formats 3 and 6, respectively.

FIGS. 3A-3D depict percent cancer cell killing in osteosarcoma (FIG. 3A) cells or melanoma cells (FIG. 3B) incubated with a dual pMHC-targeting T cell engager (squares) compared to a single pMHC-targeting T cell engager (circles). MAGE-A4 & HLA-A*02:01 positive cell line U2OS (osteosarcoma) was incubated with human PBMCs at an E:T ratio of 10:1. Cancer cell killing was measured at various concentrations of the two antigen binding proteins with an LDH release assay after 48 hours. T cell activation was determined by quantification of CD69 and CD25 markers on the CD8 T cell population after 24 h using flow cytometry (C: osteosarcoma cells; D: melanoma cells).

FIG. 4 depicts a schematic of one embodiment of the bispecific antibody of the invention. The represented embodiment, a Fab-(scFv)₂, comprises an anti-CD3 Fab fragment and two single chain antibody fragments (scFv) which specifically bind target peptides presented on MHC complexes. The pMHC binding scFvs may be linked to the C-termini of the CH1- and CL-domains via a glycine-serine flexible linker.

FIG. 5 depicts percent cancer cell killing in osteosarcoma cells incubated with a dual pMHC-targeting T cell engager (circles) compared to a single pMHC-targeting T cell engager (triangles) in Fab-(scFv)₂ and Fab-scFv formats, respectively.

FIGS. 6A-6B depict percent cell survival in lung squamous cell carcinoma (FIG. 6A) and colorectal adenocarcinoma (FIG. 6B) cells incubated with two distinct pMHC-targeting T cell engagers in mono- and dual formats.

FIG. 7A and FIG. 7B depict graphs of in vitro cell killing with antigen binding proteins with MAGE-A4 binding arms comprising two identical VHHs (FIG. 7A) or scFvs (FIG. 7B) fused to CD3 binding Fabs with low (circle), mid (square) and high (triangle) affinities.

FIGS. 8A-8B depict cytokine release in antigen-positive osteosarcoma cells incubated with three different dual pMHC-targeting T cell engagers in Fab-VHH2 format. Each engager has a different level of binding affinity for CD3 (high, mid, and low). MAGE-A4 & HLA-A*02 positive cell lines were incubated with human PBMCs at an E:T ratio of 10:1. Cytokines IL-2 (FIG. 8A) and IFN gamma (FIG. 8B) were measured at various concentrations of the three antigen binding proteins.

FIG. 9 depicts in vitro cell killing of the dual pMHC T cell engager with low (41 nM) and high (0.1 nM) affinity to the cancer antigen MAGE-A4 and equal affinity to CD3.

FIG. 10 depicts a schematic of the exemplary bispecific antibody of the invention that binds to a T cell and two pMHC targets on a tumor cell (Anti-MAGE-A4 Dual engager) and a schematic of a comparator consisting of an affinity enhanced recombinant soluble T-cell receptor (sTCR) fused to an anti-CD3 fragment. The MAGE-A4 affinity indicated for the Fab-(scFv)₂ was measured in monovalent format.

FIG. 11A depicts in vitro T cell activation in TAP-deficient T2 cells loaded with HLA-A*02:01-restricted cancer target peptide MAGE-A4 and similar physiologically relevant off-target Si (GLADGRTHTV, SEQ ID NO.: 89) and S16 (GLYDGPVHEV, SEQ ID NO.: 90) peptides upon co-culture with dual pMHC-targeting T-cell engager or a sTCRxCD3 comparator and healthy donor PBMCs. FIG. 11B depicts IFN gamma release associated with T cell activation in T2 cells loaded with cancer target peptide MAGE-A4 and similar physiologically relevant off-target Sl and S16 peptides upon co-culture with dual pMHC-targeting T-cell engager and healthy donor PBMCs.

FIGS. 12A-12D show that dual pMHC-targeting T-cell engager demonstrates limited cross-reactivity towards antigen-negative cells in vitro. Percent cytotoxicity was determined for melanoma SK-MEL-30 cells (FIG. 12A), lung adenocarcinoma NCI-H441 cells (FIG. 12B), breast cancer MDA-MB-231 cells (FIG. 12C) and pancreatic carcinoma PANC-1 cells (FIG. 12D).

FIG. 13 depicts percent cancer cell killing in osteosarcoma cells and melanoma cells incubated with different concentrations of a dual pMHC-targeting T cell engager or an affinity enhanced recombinant sTCR T cell engager comparator shown in FIG. 10. MAGE-A4 & HLA-A*02:01 positive cell lines A375 (melanoma) and U2OS (osteosarcoma) were incubated with human PBMCs at an E:T ratio of 10:1. LDH release was measured as a marker of cancer cell killing at various concentrations of the two antigen binding proteins.

FIG. 14 depicts cytokine release in osteosarcoma cells or melanoma cells co-cultured with PBMCs from healthy donors incubated with a dual pMHC-targeting T cell engager compared or the sTCR T cell engager comparator shown in FIG. 10. MAGE-A4 & HLA-A*02 positive cell lines A375 (melanoma) and U2OS (osteosarcoma) were incubated with human PBMCs at an E:T ratio of 10:1. Cytokines IL-2 and IFN gamma were quantified using ELISAs to measure the level of cytokines released in the supernatant at various concentrations of the two antigen binding proteins.

FIGS. 15A-15B depict live cell imagining of MAGE-A4 positive NCI-H1703 lung squamous carcinoma cells co-cultured with human PBMCs in presence of a dual pMHC-targeting T-cell engager (“dual pMHC TCE”) with specificity for MAGE-A4/HLA-A*02:01. FIG. 15A (left) shows lung cancer cells and PBMCs alone; FIG. 15B (right) shows lung cancer cells and PBMCs in presence of the dual pMHC TCE.

FIG. 16 depicts detection of pre-existing anti-drug antibodies (ADAs) against the comparator and an antibody devoid of pre-existing ADA epitopes. The comparator and the antibody devoid of pre-existing ADA epitopes were evaluated in serum samples from 10 healthy naïve Caucasian human donors. Pre-existing ADAs were detected by ELISA.

FIG. 17 depicts detection of pre-existing ADAs in humanized single domain antibodies (sdAb) with select modifications. “+A” corresponds to the addition of an alanine. “−S” corresponds to the deletion of a serine at position 113, according to Kabat numbering. “−SS” corresponds to the deletion of a serine at position 112 and 113, according to Kabat numbering. “SSS” corresponds to the substitution of hydrophobic amino acids at Kabat positions 11, 89, and 108 to serine amino acids. The ADA response was measured with an ELISA over different sample serum concentrations.

FIG. 18 depicts detection of pre-existing ADAs in Fab_scFv antigen binding proteins with selected modifications on the scFv binding arm. “+A” corresponds to the addition of an alanine. “−S” corresponds to the deletion of a serine at position 113, according to Kabat numbering. “−SS” corresponds to the deletion of a serine at position 112 and 113, according to Kabat numbering. “SSS” corresponds to the substitution of hydrophobic amino acids at Kabat positions 11, 89, and 108 to serine amino acids. The ADA response was measured with an ELISA over different sample serum concentrations.

### DETAILED DESCRIPTION

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of the term “including,” as well as other forms, such as “includes” and “included,” is not limiting.

So that the invention may be more readily understood, certain terms are first defined.

### Antigen Binding Proteins

As used herein, the term “antibody” or “antigen binding protein” refers to an immunoglobulin molecule or immunoglobulin derived molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)₂ fragments, Fab′ fragments, Fv fragments, recombinant IgG (rIgG) fragments, single chain variable fragments (scFv) and single domain antibodies (e.g., sdAb, sdFv, nanobody, VHH) fragments. The antibody may thus be a single domain antibody or comprise at least one variable light and at least one variable heavy chain. In one embodiment, the at least one variable light and at least one variable heavy chain are displayed as a single polypeptide chain. The term “antibody” or “antigen binding protein” includes germline derived antibodies. The term “antibody” or “antigen binding protein” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” or “antigen binding protein” should be understood to encompass functional antibody fragments thereof.

In certain embodiments, the antigen binding protein is not a T cell receptor (TCR), including but not limited to, a soluble TCR.

In certain embodiments, the antigen binding protein is multispecific (i.e., binds to two or more different target molecules or to two or more epitopes on the same target molecule). In certain embodiments, the antigen binding protein is bispecific and e.g., binds to two different target molecules or to two epitopes on the same target molecule. In certain embodiments, the antibody is trispecific and e.g., binds to at least three different target molecules.

The antigen binding protein may be monovalent or multivalent, i.e., having one or more antigen binding sites. Non-limiting examples of monovalent antigen binding proteins include scFv, Fab, scFab, dAb, VHH, V(NAR), DARPins, affilins and nanobodies. A multivalent antigen binding protein can have two, three, four or more antigen binding sites. Non-limiting examples of multivalent antigen binding proteins include full-length immunoglobulins, F(ab′)2fragments, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomABs, quadroma, CrossMab, DuoBody, triabodies and tetrabodies. In some embodiments, the multivalent antigen binding protein is bivalent, i.e., two binding sites are present. In some embodiments, the multivalent antigen binding protein is bispecific, i.e., the antigen binding protein is directed against two different targets or two different target sites on one target molecule. In some embodiments, the multivalent antigen binding protein includes more than two, e.g., three or four different binding sites for three or four, respectively, different antigens. Such antigen binding protein is multivalent and multispecific, in particular tri- or tetra-specific, respectively.

In some embodiments, the antigen binding proteins are multispecific (e.g., bispecific), such as, without being limited to, diabodies, single-chain diabodies, DARTs, BiTEs, tandem scFvs or IgG-like asymmetric heterobispecific antibodies. In certain embodiments, one or the binding specificities of the multispecific antigen binding protein is an immune cell engager (i.e., comprising binding affinity to a cell surface protein of an immune cell). Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, but are limited to, CD3, TCRα, TCRβ, CD16, NKG2D, CD89, CD64, and CD32.

In certain embodiments, the immune cell target antigen is CD3. The term CD3 refers to the cluster of differentiation 3 co-receptor (or co-receptor complex) of the T cell receptor.

As used herein, a “single-chain variable fragment” (scFv) is an antigen binding protein comprising a heavy chain variable domain (VH) linked to a light chain variable domain (VL). The VH and VL domains of the scFv are linked via any appropriate art recognized linker. Such linkers include, but are not limited to, repeated GGGGS (SEQ ID NO.: 88) amino acid sequences or variants thereof. The scFv is generally free of antibody constant domain regions, although an scFv of the disclosure may be linked or attached to antibody constant domain regions (e.g., antibody Fc domain) to alter various properties of the scFv, including, but not limited to, increased serum or tissue half-life. An scFv generally has a molecular weight of about 25 kDa and a hydrodynamic radius of about 2.5 nm.

As used herein, a “Fab fragment” or “Fab” or “Fab domain” is an antibody fragment comprising a light chain fragment comprising a variable light (VL) domain and a constant domain of the light chain (CL), and variable heavy (VH) domain and a first constant domain (CH1) of the heavy chain.

As used herein, a “VHH”, “nanobody”, “heavy-chain only antibody”, “single domain antibody”, or “sdAb” is an antigen binding protein comprising a single heavy chain variable domain derived from the species of the Camelidae family, which includes camels, llama, alpaca. A VHH generally has a molecular weight of about 15 kDa.

The antigen binding proteins of the disclosure may comprise one or more linkers for linking the domains of the antigen binding protein (e.g., linking a VH and VL to form a scFv, or linking multiple binding domains to form a multispecific antigen binding protein).

Illustrative examples of linkers include glycine polymers (Gly)ₙ; glycine-serine polymers (GlyₙSer)ₙ, where n is an integer of at least one, two, three, four, five, six, seven, or eight; glycine-alanine polymers; alanine-serine polymers; and other flexible linkers known in the art.

Glycine and glycine-serine polymers are relatively unstructured, and therefore may be able to serve as a neutral tether between domains of fusion proteins such as the antigen binding proteins described herein. Glycine accesses significantly more phi-psi space than other small side chain amino acids, and is much less restricted than residues with longer side chains (Scheraga, Rev. Computational Chem. 1: 1173-142 (1992)). A person skilled in the art will recognize that design of an antigen binding protein in particular embodiments can include linkers that are all or partially flexible, such that the linker can include flexible linker stretches as well as one or more stretches that confer less flexibility to provide a desired structure.

Linker sequences can however be chosen to resemble natural linker sequences, for example, using the amino acid stretches corresponding to the beginning of human CH1 and Cκ sequences or amino acid stretches corresponding to a portion of the hinge region of human IgG.

The design of the peptide linkers connecting VL and VH domains in the scFv moieties are flexible linkers generally composed of small, non-polar or polar residues such as, e.g., Gly, Ser and Thr. A particularly exemplary linker connecting the variable domains of the scFv moieties is the (Gly₄Ser)₄ linker, where 4 is the exemplary number of repeats of the motif.

Linkers connecting the scFv antigen binding proteins to the Fab domain are also envisioned. In certain embodiments, the scFv antigen binding proteins are linked to the CH1 and CL domains of the Fab with a Gly-Ser linker. In certain embodiments, the linker comprises the amino acid sequence GGGGS (SEQ ID NO.: 88).

Other exemplary linkers include, but are not limited to the following amino acid sequences: GGG; DGGGS (SEQ ID NO.: 91); TGEKP (SEQ ID NO.: 92) (Liu et al, Proc. Natl. Acad. Sci.94: 5525-5530 (1997)); GGRR (SEQ ID NO.: 93); (GGGGS)ₙ(SEQ ID NO.: 88) wherein n=1, 2, 3, 4 or 5 (Kim et al, Proc. Natl. Acad. Sci.93: 1156-1160 (1996)); EGKSSGSGSESKVD (SEQ ID NO.: 94) (Chaudhary et al., Proc. Natl. Acad. Sci. 87: 1066-1070 (1990)); KESGSVSSEQLAQFRSLD (SEQ ID NO.: 95) (Bird et al., Science 242:423-426 (1988)), GGRRGGGS (SEQ ID NO.: 96); LRQRDGERP (SEQ ID NO.: 97); LRQKDGGGSERP (SEQ ID NO.: 98); and GSTSGSGKPGSGEGSTKG (SEQ ID NO.: 99) (Cooper et al, Blood, 101(4): 1637-1644 (2003)). Alternatively, flexible linkers can be rationally designed using a computer program capable of modeling the 3D structure of proteins and peptides or by phage display methods.

The antibodies may comprise a variable light (VL) domain and a variable heavy (VH) domain. Each VL and VH domain further comprises a set of three CDRs.

As used herein, the term “complementarity determining region” or “CDR” refers to non-contiguous sequences of amino acids within antibody variable regions, which confer antigen specificity and binding affinity. In general, there are three CDRs in each heavy chain variable domain (CDRH1, CDRH2, CDRH3) and three CDRs in each light chain variable domain (CDRL1, CDRL2, CDRL3). “Framework regions” or “FRs” are known in the art to refer to the non-CDR portions of the variable domains of the heavy and light chains. In general, there are four FRs in each heavy chain variable domain (HFR1, HFR2, HFR3, and HFR4), and four FRs in each light chain variable domain (LFR1, LFR2, LFR3, and LFR4). Accordingly, an antibody variable region amino acid sequence can be represented by the formula FR1-CDR1-FR2-CDR2-FR3-CDR3-FR4. Each segment of the formula, i.e., FR1, CDR1, FR2, CDR2, FR3, CDR3, and FR4, represents a discrete amino acid sequence (or a polynucleotide sequence encoding the same) that can be mutated, including one or more amino acid substitutions, deletions, and insertions. In certain embodiments, an antibody variable light chain amino acid sequence can be represented by the formula LFR1-CDRL1-LFR2-CDRL2-LFR3-CDRL3-LFR4. In certain embodiments, an antibody variable heavy chain amino acid sequence can be represented by the formula HFR1-CDRH1-HFR2-CDRH2-HFR3-CDRH3-HFR4.

The precise amino acid sequence boundaries of a given CDR or FR can be readily determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists exemplary position boundaries of CDRL1, CDRL2, CDRL3 and CDRH1, CDRH2, CDRH3 of an antibody, as identified by Kabat, Chothia, and Contact schemes, respectively. For CDRH1, residue numbering is listed using both the Kabat and Chothia numbering schemes. CDRs are located between FRs, for example, with CDRL1 located between LFR1 and LFR2, and so forth. It is noted that because the shown Kabat numbering scheme places insertions at H35A and H35B, the end of the Chothia CDRH1 loop when numbered using the shown Kabat numbering convention varies between H32 and H34, depending on the length of the loop.

TABLE 1
Exemplary Position Boundaries of CDRs

| CDR | Kabat | Chothia | Contact |
|---|---|---|---|
| LCDR1 | L24-L34 | L24-L34 | L30-L36 |
| LCDR2 | L50-L56 | L50-L56 | L46-L55 |
| LCDR3 | L89-L97 | L89-L97 | L89-L96 |
| HCDR1 | H31-H35B | H26-H32 . . . 34 | H30-H35B |
| (Kabat Numbering¹) | | | |
| HCDR1 | H31-H35 | H26-H32 | H30-H35 |
| (Chothia Numbering²) | | | |
| HCDR2 | H50-H65 | H52-H56 | H47-H58 |
| HCDR3 | H95-H102 | H95-H102 | H93-H101 |

¹Kabat et al. (1991), “Sequences of Proteins of Immunological Interest,” 5^{th} Ed. Public Health Service, National Institutes of Health, Bethesda, MD
²Al-Lazikani et al. (1997), J. Mol. Biol. 273: 927-948

Thus, unless otherwise specified, a “CDR” or “complementary determining region,” or individual specified CDRs (e.g., CDRH1, CDRH2), of a given antibody or fragment thereof, such as a variable domain thereof, should be understood to encompass a (or the specific) complementary determining region as defined by any of the known schemes. Likewise, unless otherwise specified, an “FR” or “framework region,” or individual specified FRs (e.g., “HFR1,” “HFR2”) of a given antibody or fragment thereof, such as a variable domain thereof, should be understood to encompass a (or the specific) framework region as defined by any of the known schemes. In some instances, the scheme for identification of a particular CDR or FR is specified, such as the CDR as defined by the Kabat, Chothia, or Contact method. In other cases, the particular amino acid sequence of a CDR or FR is given.

In certain embodiments, the antigen binding proteins disclosed here are rabbit antigen binding proteins or rabbit-derived antigen binding proteins. In certain embodiments, the rabbit antigen binding proteins are humanized. As used herein, the term “humanized” or “humanization” refers to an antigen binding protein that has been altered to make it more like a human antibody. Non-human antigen binding proteins, such as rabbit antigen binding proteins, would elicit a negative immune reaction if administered to a human for therapy. It is therefore advantageous to humanize the rabbit antigen binding proteins for later therapeutic use.

In certain embodiments, the antigen binding proteins are humanized through resurfacing (i.e., remodel the solvent-.accessible residues of the non-human framework such that they become more human-like), Resurfacing strategics are described in more detail in WO2004/016740, WO2008/144757, and WO2005/016950, each of which is incorporated herein by rtiftrence.

In certain embodiments, the antigen binding proteins are humanized through CDR grafting (i.e., inserting the rabbit antigen binding protein CDRs into a human antibody acceptor framework). Grafting strategies and human acceptor frameworks are described in more detail in WO2009/155726, incorporated herein by reference.

As used herein, the term “affinity” (or “binding affinity” as used interchangeably herein) refers to the strength of the interaction between an antibody's antigen binding site and the epitope to which it binds. As readily understood by those skilled in the art, an antibody or antigen binding protein affinity may be reported as an equilibrium dissociation constant (K_{D}) in molarity (M). The equilibrium dissociation constant K_{D} is calculated from the association rate constant kₐ(having the unit M⁻¹s⁻¹) and the dissociation rate constant k_{d} (having the unit s⁻¹) by k_{d}/kₐ. The antibodies of the disclosure may have K_{D} values in the range of 10⁻⁷ to 10⁻¹⁴ M. High affinity antibodies have K_{D} values of 10⁻⁹ M (1 nanomolar, nM) and lower. For example, a high affinity antibody may have a K_{D} value in the range of about 1 nM to about 0.01 nM. A high affinity antibody may have K_{D} value of about 1 nM, about 0.9 nM, about 0.8 nM, about 0.7 nM, about 0.6 nM, about 0.5 nM, about 0.4 nM, about 0.3 nM, about 0.2 nM, or about 0.1 nM. Very high affinity antibodies have K_{D} values of 10⁻¹² M (1 picomolar, pM) and lower. Weak, or low, affinity antibodies may have K_{D} values in the range of 10⁻¹ to 10⁻⁴ M. Low affinity antibodies may have K_{D} values of 10⁻⁴ M and higher, such as 10⁻⁴ M, 10⁻³ M, 10⁻² M, or 10⁻¹ M. The ability of an antibody to bind to a specific antigenic determinant (e.g., a target peptide-MHC) can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). Generally, binding parameters can be determined at temperatures in the range of 20° C. to 30° C. The present specification makes reference to binding parameters measured by SPR throughout. Typically, in embodiments pertaining to each reference to SPR throughout the present specification, association rate constant values, dissociation rate constant values and equilibrium dissociation constant values recited herein are determined by SPR at 25° C. Preferably, the SPR-based system used is the BIAcore SPR system. The skilled person will appreciate that the binding parameters can be measured in the context of the monovalent or bivalent bi-, tri- or multispecific constructs; preferably, the parameters are determined in the context of the whole construct.

As used herein, the term “T cell receptor” or “TCR” refers to a heterodimeric protein comprised of two different chains (TCRα and TCRβ), which structurally belong to the immunoglobulin (Ig) superfamily. The extracellular portion of each chain is composed of variable (“Vα” and “Vβ”) and constant (“Cα” and “Cβ”) domains, and a hinge region, where the formation of a stabilizing disulfide bond occurs. The intracellular region forms a non-covalent interaction with another trans-membrane protein, CD3, which in the case of the correct target recognition leads to a series of conformational changes and a first T cell activation signal. Recognition and binding of peptide-MHC (Pmhc) by a TCR is governed by the six hypervariable loops, termed complementarity determining regions (CDRs), located on the variable domains of the TCRα (CDRα1, CDRα2, CDRα3) and TCRβ (CDRβ1, CDRβ2, CDRβ3). CDR3 loops (CDRα3 and CDRβ3) lead the recognition of the processed antigen with the support of CDRα1 and CDRβ1, that have been implicated in the recognition of the N- and C-terminal amino acids of the presented peptide, respectively (Rudolph et al. Annu Rev Immunol. 24:419-66. 2006). Recognition of the MHC is typically achieved through the interaction with CDRα2 and CDRβ2. The high sequence diversity of the TCR is achieved through V(D)J recombination process, in which the variable domain is generated from a combination of genes: V (variable) and J (joining) for both TCRα and TCRβ, and an additional D (diversity) gene for TCRβ. The high antigen specificity of the TCR is controlled by the thymic maturation process, in which the self-reacting T cells are negatively selected. TCR affinity towards the specific pMHC and the functional avidity are the key factors controlling T-cell activation. A critical role in antigen recognition, however, is played by the affinity (K_{D}), i.e., the strength of binding between the TCR and the cell-displayed pMHC (Tian et al. J Immunol. 179:2952-2960. 2007). The physiological affinities of TCRs range from 1 mM to 100 mM (Davis et al. Annu Rev Immunol. 16:523-544. 1998), which, in comparison to antibodies, is relatively low.

As used herein, the term “peptide-MHC” refers to a major histocompatibility complex (MHC) molecule (MHC-I or -II) with an antigenic peptide bound in a peptide binding pocket of the MHC. In certain embodiments, the MHC is a human MHC.

### Dual Peptide-MHC—Immune Cell Engaging Antigen Binding Proteins

Certain antigen binding proteins described herein possess at least two pMHC binding domains and a binding domain with binding specificity to a cell surface protein of an immune cell (e.g., CD3 on the surface of a T cell; an “immune cell binding domain”). Targeting two pMHC complexes on the surface of a target cell (e.g., a cancer cell) improves target cell engagement through avidity-enhanced binding. The enhanced binding (i.e., lower apparent K_{D} of the multivalent interaction) may in turn promote improved target cell killing relative to an antigen binding protein that only has one pMHC binding domain. The avidity-enhanced binding created by at least two pMHC binding domains may be particularly useful when targeting pMHC complexes of low copy number on the surface of a target cell (e.g., cancer cell).

In one aspect, the disclosure provides an antigen binding protein comprising: a) a Fab domain which specifically binds to a cell surface protein of an immune cell, the Fab domain comprising a heavy chain and a light chain; b) at least a first pMHCbinding domain operably linked to the heavy chain, wherein the first pMHC binding domain binds to first target peptide-MHC (pMHC) complex; and c) at least a second pMHC binding domain operably linked to the light chain, wherein the second pMHC binding domain binds to a second pMHC complex.

In certain embodiments, the Fab domain heavy chain comprises a CH1 domain and a VH domain. In certain embodiments, the Fab domain further comprises at least 5 amino acids of an antibody hinge region, in particular at the C-terminus of the heavy chain of the Fab domain. In certain embodiments, said Fab domain comprises up to, or at most, 10 amino acids of an antibody hinge region. In certain embodiments, the Fab domain comprises the sequence stretch up to the first cysteine of the antibody hinge region. In certain embodiments, said sequence stretch is or comprises the sequence EPKSC (SEQ ID NO.: 87). The presence of cysteine allows for an additional disulfide bridge which may further stabilize the antigen binding protein. In some embodiments, said at most 10 amino acids of an antibody hinge region comprises EPKSCDKTHT (SEQ ID NO.: 100). The antibody hinge region may additionally comprise the sequence GGGGS (SEQ ID NO.: 88) which may serve as a linker sequence to the pMHC binding domain(s). Thus, in some embodiments, a pMHC binding domain is linked to the C-terminal end of the Fab CH1 domain via any of EPKSCGGGGS (SEQ ID NO.: 101), EPKSCDKTHT (SEQ ID NO.: 100), EPKSCDKTHTGGGGS (SEQ ID NO.: 102), DKTHT (SEQ ID NO.: 103), DKTHTGGGGS (SEQ ID NO.: 104) or GGGGSGGGGS (SEQ ID NO.: 105) linker.

In certain embodiments, the Fab domain light chain comprises a CL domain and a VL domain. The CL domain may be followed by a linker, such as GGGGS (SEQ ID NO.: 88).

Suitable linker sequences between the immune cell binding domain and the pMHC binding domains include glycine polymers (Gly)n; glycine-serine polymers (GlynSer)y, wherein n and y are an integer of at least one, two, three, four, five, six, seven, or eight; glycine-alanine polymers; alanine-serine polymers; and other flexible linkers known in the art. In various embodiments, the linker sequence connecting the immune cell binding domain and the pMHC binding domain(s) is the (Gly₄Ser)₁ (SEQ ID NO.: 88) linker sequence.

In some embodiments, the antigen binding protein of the present disclosure comprises at least 5 amino acids of an antibody hinge region (in certain embodiments the sequence stretch up to the first cysteine of an antibody hinge region), such as 5-10 amino acids or at most 10 amino acids located at the C-terminus of the heavy chain of the Fab domain, and further comprises a sequence that follows the said at least 5 amino acids of an antibody hinge region and that serves as a linker connecting a first or second pMHC domain as described elsewhere herein. Preferably, the at least 5 amino acids of an antibody hinge region comprise the sequence EPKSC (SEQ ID NO.: 87), or comprise the sequence EPKSCDKTHT (SEQ ID NO.: 100), and the sequences that serve as a linker connecting the first or second pMHC binding domain comprise the linker sequences as described above. In some embodiments, the at least 5 amino acids of an antibody hinge region comprise the sequence EPKSC (SEQ ID NO.: 87), or comprise the sequence EPKSCDKTHT (SEQ ID NO.: 100), and the sequences that serve as a linker connecting the first or second pMHC binding domain comprise the sequence GGGGS (SEQ ID NO.: 88).

Linker sequences connecting the variable domains of an scFv may include glycine polymers (Gly)n; glycine-serine polymers (GlynSer)y, where n and y are integers of at least one, two, three, four, five, six, seven, or eight; glycine-alanine polymers; alanine-serine polymers; and other flexible linkers known in the art. In certain embodiments, the linker sequence is a glycine-serine linker sequence (GlynSer)y, where n and y are an integers of at least one, two, three, four, five, six, seven, or eight. In certain embodiments thereof, the linker sequence connecting the variable domains of an scFv is the (Gly₄Ser)₄ (SEQ ID NO.: 106) linker sequence.

In certain embodiments, the antigen binding protein does not comprise an Fc domain. In certain embodiments thereof, such antigen binding protein lacking an Fc domain is a Fab-sdAb, a Fab-(sdAb)₂, a Fab-scFv or a Fab-(scFv)₂, a F(ab′)₂fragment, a bis-scFv (or tandem scFv or BiTE), a DART, diabodies, a scDb, a triabody, a tetrabody, or MATCH.

In certain embodiments, the first target pMHC complex and the second target pMHC complex are the same (i.e., each complex comprises the same peptide bound to the MHC molecule). In certain embodiments, the first pMHC binding domain and the second pMHC binding domain are the same (i.e., the binding domains bind to the same epitope).

In certain embodiments, the first target pMHC complex and the second target pMHC complex are different (i.e., each complex comprises a different peptide bound to the MHC molecule). In certain embodiments, the first pMHC binding domain and the second pMHC binding domain are different (i.e., the binding domains bind to different epitopes).

In certain embodiments, the first pMHC binding domain is operably linked to the C-terminus of the Fab heavy chain or the N-terminus of the Fab heavy chain. In certain embodiments, the first pMHC binding domain is operably linked to the C-terminus of the Fab light chain or the N-terminus of the Fab light chain.

In certain embodiments, the second pMHC binding domain is operably linked to the C-terminus of the Fab heavy chain or the N-terminus of the Fab heavy chain. In certain embodiments, the second pMHC binding domain is operably linked to the C-terminus of the Fab light chain or the N-terminus of the Fab light chain.

In certain embodiments, the pMHC binding domain is a scFv or an sdAb. As described elsewhere herein, the pMHC binding domain may also be any one of a scFab, a diabody, or a Fab. As described elsewhere herein and as exemplified in the Examples and the drawings, the pMHC binding domain is in particular a scFv or a sdAb (VHH), more particularly each of the at least first pMHC binding domain and/or each of the at least second pMHC binding domain is a scFv or a sdAb (VHH). Further, in accordance with the experimental data, in certain embodiments, both the at least first pMHC binding domain and the at least second pMHC binding domain are each a scFv, or are each a sdAb, and both the at least first pMHC binding domain and the at least second pMHC binding domain are the same. Still further, and as exemplified in the Examples and the drawings, in certain embodiments thereof, the antigen binding protein is bivalent for the target pMHC complex and comprises no more than two pMHC binding domains and both said pMHC binding domains are targeting the same pMHC complex.

As described elsewhere herein, and as follows from the above, the at least first and the at least second pMHC binding domain may both be linked to either the heavy chain, or may both be linked to the light chain of the Fab domain. As further described elsewhere herein and as exemplified in the Examples and the drawings, in some embodiments, the at least first and the at least second pMHC binding domain are not linked to the same chain of the Fab domain, i.e., one is linked to the heavy chain of the Fab domain, and the other is linked to the light chain of the Fab domain. As described elsewhere herein, in certain embodiments,
(i) the at least first pMHC binding domain is operably linked to the C-terminus of the heavy chain of the Fab domain, and the at least second pMHC binding domain is operably linked to the C-terminus of the light chain of the Fab domain, or
(ii) the at least first pMHC binding domain is operably linked to the C-terminus of the heavy chain of the Fab domain, and the at least second pMHC binding domain is operably linked to the N-terminus of the light chain of the Fab domain, or
(iii) the at least first pMHC binding domain is operably linked to the N-terminus of the heavy chain of the Fab domain, and the at least second pMHC binding domain is operably linked to the N-terminus of the light chain of the Fab domain, or
(iv) the at least first pMHC binding domain is operably linked to the N-terminus of the heavy chain of the Fab domain, and the at least second pMHC binding domain is operably linked to the C-terminus of the light chain of the Fab domain, or
(v) the at least first pMHC binding domain is operably linked to the N-terminus of the heavy chain of the Fab domain, and the at least second pMHC binding domain is operably linked to the C-terminus of the heavy chain of the Fab domain, or
(vi) the at least first pMHC binding domain is operably linked to the N-terminus of the light chain of the Fab domain, and the at least second pMHC binding domain is operably linked to the C-terminus of the light chain of the Fab domain, or
(vii) both the at least first pMHC binding domain and the at least second pMHC binding domain are operably linked to the C-terminus or to the N-terminus of the light chain of the Fab domain, or
(viii) both the at least first pMHC binding domain and the at least second pMHC binding domain are operably linked to the C-terminus or to the N-terminus of the heavy chain of the Fab domain.

In certain embodiments, the antigen binding protein comprises:
1) a first pMHC binding scFv linked to the C-terminus of the Fab domain heavy chain and a second pMHC binding scFv linked to the C-terminus of the Fab domain light chain;
2) a first pMHC binding scFv linked to the N-terminus of the Fab domain heavy chain and a second pMHC binding scFv linked to the N-terminus of the Fab domain light chain;
3) a first pMHC binding scFv linked to the N-terminus of the Fab domain heavy chain and a second pMHC binding scFv linked to the C-terminus of the Fab domain light chain;
4) a first pMHC binding scFv linked to the C-terminus of the Fab domain heavy chain and a second pMHC binding scFv linked to the N-terminus of the Fab domain light chain;
5) a first pMHC binding sdAb linked to the N-terminus of the Fab domain heavy chain and a second pMHC binding sdAb linked to the N-terminus of the Fab domain light chain;
6) a first pMHC binding sdAb linked to the C-terminus of the Fab domain heavy chain and a second pMHC binding sdAb linked to the C-terminus of the Fab domain light chain;
7) a first pMHC binding sdAb linked to the N-terminus of the Fab domain heavy chain and a second pMHC binding sdAb linked to the C-terminus of the Fab domain light chain; or
8) a first pMHC binding sdAb linked to the C-terminus of the Fab domain heavy chain and a second pMHC binding sdAb linked to the N-terminus of the Fab domain light chain.

As described elsewhere herein and as exemplified in the Examples and the drawings, in certain embodiments, (i) the at least first pMHC binding domain is operably linked to the C-terminus of the heavy chain of the Fab domain, and the at least second pMHC binding domain is operably linked to the C-terminus of the light chain of the Fab domain, or (ii) the at least first pMHC binding domain is operably linked to the C-terminus of the heavy chain of the Fab domain, and the at least second pMHC binding domain is operably linked to the N-terminus of the light chain of the Fab domain. As further described elsewhere herein and as further exemplified in the Examples and the drawings, in certain embodiments thereof, such antigen binding protein has no more than two pMHC binding domains, i.e., is limited with regard to pMHC binding domains to one first pMHC binding domain and one second pMHC binding domain. In certain embodiments thereof, the antigen binding protein is bivalent for the target pMHC complex. Accordingly, in such embodiments, the antigen binding protein comprises no more than two pMHC binding domains, which are both binding to a pMHC complex, which comprises the same target peptide bound to/presented by the MHC molecule. As further described elsewhere herein and as further exemplified in the Examples and the drawings, such antigen binding protein is bispecific, and has preferably binding specificity for CD3. In the aforementioned embodiments, it is further preferred that the two pMHC binding domains are both scFv, or are both sdAb (VHH). Accordingly, the present disclosure encompasses, in certain embodiments, a bispecific bivalent antigen binding protein, comprising a Fab domain which specifically binds to CD3; and no more than two pMHC binding domains, wherein both pMHC binding domains are targeting the same pMHC complex (i.e., the antigen binding protein is bivalent with regard to the target pMHC complex, wherein both pMHC binding domains are each a scFv, or are each a sdAb (VHH), and wherein (i) one of the two pMHC binding domains is operably linked to the C-terminus of the heavy chain of the CD3 binding domain, and the other pMHC binding domain is operably linked to the C-terminus of the light chain of the CD3 binding domain, or (ii) one of the two pMHC binding domains is operably linked to the C-terminus of the heavy chain of the CD3 binding domain, and the other pMHC binding domain is operably linked to the N-terminus of the light chain of the CD3 binding domain. As described elsewhere herein, the two pMHC binding domains may also be any one of a scFab, a diabody or a Fab.

As further described elsewhere herein and as further exemplified in the Examples and the drawings, in some embodiments, the antigen binding protein has no more than two pMHC binding domains, i.e., is limited with regard to pMHC binding domains to one first pMHC binding domain one second pMHC binding domain, in particular to pMHC binding domains which are both scFv and are the same (or are both sdAb and the same), and wherein one is operably linked to the heavy chain of the Fab domain, and the other is operably linked to the light chain of the Fab domain, wherein it is even more preferred that (i) one of the two pMHC binding domains is operably linked to the C-terminus of the Fab domain heavy chain, and the other pMHC binding domain is operably linked to the C-terminus of the Fab domain light chain, or (ii) one of the two pMHC binding domains is operably linked to the C-terminus of the Fab domain heavy chain, and the other pMHC binding domain is operably linked to the N-terminus of the Fab domain light chain.

In certain embodiments, the first pMHC binding domain and/or the second pMHC binding domain comprise a variable heavy chain having a polar amino acid at position 11, 89 and/or 108, according to Kabat numbering. The presence of polar amino acids at the indicated positions may reduce anti-drug antibodies.

In certain embodiments, the immune cell binding domain, such as the Fab domain and/or the CD3 binding domain described elsewhere herein, comprises a variable heavy chain having a non-polar amino acid at position 11, 89 and/or 108, according to Kabat numbering.

In certain embodiments, the variable heavy chain comprises: leucine (L) or serine (S) at amino acid position 11, according to Kabat numbering; valine (V), serine (S), or threonine (T) at amino acid position 89, according to Kabat numbering; and/or leucine (L), serine (S), or threonine (T) amino acid position 108, according to Kabat numbering.

In certain embodiments, when leucine (L) is present at amino acid position 11, then serine (S) or threonine (T) are present at amino acid position 89, and serine (S) or threonine (T) are present at amino acid position 108, according to Kabat numbering.

In certain embodiments, when valine (V) is present at amino acid position 89, then serine (S) is present at amino acid position 11, and serine (S) or threonine (T) are present at amino acid position 108, according to Kabat numbering.

In certain embodiments, when leucine (L) is present at amino acid position 108, then serine (S) or threonine (T) are present at amino acid position 11, and serine (S) or threonine (T) are present at amino acid position 89, according to Kabat numbering.

In certain embodiments, the polar amino acid is serine (S) and/or threonine (T).

In certain embodiments, the variable heavy chain comprises serine (S) at amino acid position 11, serine (S) or threonine (T) at amino acid position 89, and serine (S) or threonine (T) at amino acid position 108, according to Kabat numbering.

In certain embodiments, the variable heavy chain comprises serine (S) at amino acid position 11, serine (S) at amino acid position 89, and serine (S) at amino acid position 108, according to Kabat numbering.

In certain embodiments, the immune cell binding domain, in particular when not comprising a CH domain, i.e., not being a Fab domain, but e.g. a scFv or a sdAb, comprises a variable heavy chain having a serine (S) at position 113 deleted, according to Kabat numbering.

In certain embodiments, the first pMHC binding domain and/or the second pMHC binding domain comprise a variable heavy chain having a serine (S) at position 113 deleted, according to Kabat numbering.

In certain embodiments, the immune cell binding domain, in particular when not comprising a CH domain, i.e., not being a Fab domain, but e.g. a scFv or a sdAb, comprises a variable heavy chain having a serine (S) at position 112 deleted and a serine (S) at position 113 deleted, according to Kabat numbering. In certain embodiments, the first pMHC binding domain and/or the second pMHC binding domain comprise a variable heavy chain having a serine (S) at position 112 deleted and a serine (S) at position 113 deleted, according to Kabat numbering.

In certain embodiments, the antigen binding protein comprises an S113A, S113G, or S113T substitution, according to Kabat numbering.

In certain embodiments, the antigen binding protein comprises an S113A, S113G, or S113T substitution, and wherein S112 is deleted, according to Kabat numbering.

In certain embodiments, the antigen binding protein comprises an S112A, S112G, or S112T substitution, according to Kabat numbering.

In certain embodiments, the antigen binding protein comprises an S112A, S112G, or S112T substitution, and wherein S113 is deleted, according to Kabat numbering.

pMHC binding domains may e.g., be generated using the library approach as described in WO2022190007A1, which is hereby incorporated by reference.

In certain embodiments, the target pMHC binding domain specifically targets an MHC restricted peptide derived from a tumor antigen or a viral antigen.

In accordance with the present disclosure, an antigen binding protein as provided by the present disclosure, in particular the at least first and/or the at least second pMHC binding domain, is highly selective and does not bind to a different pMHC complex, such as a pMHC complex presenting a different peptide.

In certain embodiments, the cell surface protein of an immune cell is selected from the group consisting of CD3, TCRα, TCRβ, CD16, NKG2D, CD89, CD64, and CD32a. In certain embodiments, the cell surface protein of an immune cell is CD3 (cluster of differentiation 3 co-receptor (or co-receptor complex) of the T cell receptor). The CD3 protein complex is composed of four distinct chains. In mammals, the complex contains a CD3γ (gamma) chain/subunit, a CD3δ (delta) chain/subunit, and two CD3ε (epsilon) chains/subunits. Reference to CD3 as the cell surface protein of an immune cell is made herein throughout, and CD3 is the particularly preferred cell surface protein as exemplified, inter alia, by the Examples. Disclosed herein is that in various aspects and embodiments that are described throughout the specification and that are pertaining to the immune cell binding domain, in particular the Fab domain, that specifically binds to CD3 on the surface of an immune cell, in particular a T cell), the Fab domain may specifically bind to the CD3γ (gamma) domain/subunit, the CD3δ (delta) chain/subunit, and/or a CD3ε (epsilon) chain/subunit of CD3. Preferably, the immune cell binding domain may specifically bind to a CD3ε (epsilon) chain/subunit of CD3.

Suitable anti-CD3 binding domains are known in the art, particularly T-cell activating CD3-epsilon binding domains. The terms “CD3 binding domain” and “anti-CD3 binding domain” are used interchangeably herein. In certain embodiments of the present disclosure, the anti-CD3 binding domain is any one of antibodies SP34, Okt3 or UCHT1, or a variant sequence thereof having at least about 80%, 85%, 90%, 95%, 96%, 97%, 98% or 99% identity thereto, while retaining the same specificity as its parent. SP34, Okt3 or UCHT1 are murine antibodies; for therapeutic applications, humanized versions of SP34, Okt3 or UCHT1, i.e., huSP34, huOkt3 or huUCHT1, are preferred. In certain embodiments, the humanized variant sequence of SP34, Okt3 or UCHT1 is optimized for use in Fab format. For example, the humanized huSP34, huOkt3 or huUCHT1 may comprise 1, 2, 3, 4, 5, 6, 7, 8, 9 or more substitutions while retaining selective binding to CD3. Exemplary CD3 binding domains are disclosed in U.S. Pat. No. 6,750,325, WO2008079713, U.S. Pat. No. 7,635,475, WO2005040220, U.S. Pat. No. 7,728,114, WO9404679, U.S. Pat. No. 7,381,803, WO2008119567, WO2014110601, WO2014145806, WO2015095392, WO2016086189 and/or WO2019195535A1, each of which is incorporated herein by reference. In certain embodiments, the immune cell is selected from the group consisting of a T cell, a B cell, a natural killer (NK) cell, a natural killer T (NKT) cell, a neutrophil cell, a monocyte, and a macrophage. In certain embodiments, the immune cell is a T cell.

In one embodiment, the anti-CD3 binding domain comprises the HCDR sequences of SEQ ID NOs.: 76, 77 and 78 and the LCDR sequences of SEQ ID NOs.: 79, 81 and 82 or a variant sequence thereof, having 1, 2 or 3 substitutions while retaining specific antigen binding. In one embodiment, the LCDR1 sequence comprises 1 substitution and is SEQ ID NO.: 80.

In one embodiment, the anti-CD3 binding domain comprises the HCDR sequences of SEQ ID NOs.: 76, 77 and 78 and the LCDR sequences of SEQ ID NOs.: 80, 81 and 82 or a variant sequence thereof, having 1, 2 or 3 substitutions while retaining specific antigen binding. In one embodiment, the LCDR1 sequence comprises 1 substitution and is SEQ ID NO.: 79.

In one embodiment, the anti-CD3 binding domain comprises the VL sequence of SEQ ID NO.: 83 and the VH sequence of SEQ ID NO.: 84 or a variant sequence thereof, being at least about 80%, 85%, 90%, 95%, 96%, 97%, 98%, 99% or 100% identical to said amino acid sequences while retaining specific antigen binding. In certain embodiments, the variant sequence comprises 1, 2, 3, 4, 5, 6, 7, 8, 9, or 10 substitutions with regard to the parental amino acid sequence, such as e.g., 1 substitution in the VL sequence and 4 substitutions in the VH sequence.

In one embodiment, the anti-CD3 binding domain comprises the VL sequence of SEQ ID NO.: 85 and the VH sequence of SEQ ID NO.: 86 or a variant sequence thereof, being at least about 80%, 85%, 90%, 95%, 96%, 97%, 98%, 99% or 100% identical to said amino acid sequences while retaining specific antigen binding. In certain embodiments, the variant sequence comprises 1, 2, 3, 4, 5, 6, 7, 8, 9, or 10 substitutions with regard to the parental amino acid sequence, such as e.g., 1 substitution in the VL sequence and 4 substitutions in the VH sequence.

Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 79, 81 and 82 or variants thereof; and no more than two pMHC binding domains targeting the same pMHC complex, wherein both pMHC binding domains are each a scFv, such as e.g., a Fab-(scFv)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 80, 81 and 82 or variants thereof; and no more than two pMHC binding domains targeting the same pMHC complex, wherein both pMHC binding domains are each a scFv, such as e.g., a Fab-(scFv)₂. Accordingly, the present disclosure encompasses, in certain embodiments, a bispecific bivalent antigen binding protein, comprising an anti-CD3-binding domain comprising the VL sequence of SEQ ID NO.: 83 and the VH sequence of SEQ ID NO.: 84 or variants thereof; and no more than two pMHC binding domains targeting the same pMHC complex, wherein both pMHC binding domains are each a scFv, such as e.g., a Fab-(scFv)₂. Accordingly, the present disclosure encompasses, in certain embodiments, a bispecific bivalent antigen binding protein, comprising an anti-CD3-binding domain comprising the VL sequence of SEQ ID NO.: 85 and the VH sequence of SEQ ID NO.: 86 or variants thereof; and no more than two pMHC binding domains targeting the same pMHC complex, wherein both pMHC binding domains are each a scFv, such as e.g., a Fab-(scFv)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 79, 81 and 82 or variants thereof; and no more than two pMHC binding domains targeting the same pMHC complex, wherein both pMHC binding domains are each a sdAb (VHH), such as e.g., a Fab-(sdAb)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 80, 81 and 82 or variants thereof; and no more than two pMHC binding domains targeting the same pMHC complex, wherein both pMHC binding domains are each a sdAb (VHH), such as e.g., a Fab-(sdAb)₂. Accordingly, the present disclosure encompasses, in certain embodiments, a bispecific bivalent antigen binding protein, comprising an anti-CD3-binding domain comprising the VL sequence of SEQ ID NO.: 83 and the VH sequence of SEQ ID NO.: 84 or variants thereof; and no more than two pMHC binding domains targeting the same pMHC complex, wherein both pMHC binding domains are each a sdAb (VHH), such as e.g., a Fab-(sdAb)₂. Accordingly, the present disclosure encompasses, in certain embodiments, a bispecific bivalent antigen binding protein, comprising an anti-CD3-binding domain comprising the VL sequence of SEQ ID NO.: 85 and the VH sequence of SEQ ID NO.: 86 or variants thereof; and no more than two pMHC binding domains targeting the same pMHC complex, wherein both pMHC binding domains are each a sdAb (VHH), such as e.g., a Fab-(sdAb)₂.

In certain embodiments, the immune cell binding domain, in particular the Fab domain, specifically binds to CD3 with a binding affinity (K_{D}) between about 1 nM to about 150 nM (e.g., 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM), as determined by SPR. In certain embodiments, the immune cell binding domain, in particular the Fab domain, specifically binds to CD3 with a binding affinity (K_{D}) between about 1 nM to about 50 nM, as determined by SPR. In certain embodiments, the immune cell binding domain, in particular the Fab domain, specifically binds to CD3 with a binding affinity (K_{D}) between about 20 nM to about 50 nM, as determined by SPR.

In certain embodiments, the immune cell binding domain, in particular the Fab domain, specifically binds to CD3 with a binding affinity (K_{D}) of about 1 nM, of about 10 nM, or of about 50 nM, as determined by SPR.

In some embodiments, the association rate constant kₐ of the anti-CD3 binding domain is between about 1×10⁵ to about 1×10⁷ M⁻¹s⁻¹, such as at least 1×10⁶ M⁻¹s⁻¹ or at least 2×10⁶ M⁻¹s⁻¹.

In some embodiments, the dissociation rate constant k_{d} of the anti-CD3 binding domain is between about 1×10⁻¹ to about 1×10⁻⁶ s⁻¹, such as at least 2×10⁻³ s⁻¹, or at least 3×10⁻³ s⁻¹ or at least 4×10⁻³ s⁻¹. Without being bound to theory, a fast dissociation rate, e.g., a k_{d}-value of 2-3×10⁻³ s⁻¹, may lead to less T cell overactivation and in consequence, less cytokine release.

In one embodiment, the association rate constant kₐ and/or the dissociation rate constant k_{d} are equivalent or similar for both CD3-heterodimers CD3ε γ (epsilon/gamma) and CD3ε 6 (epsilon/delta), i.e., there is no significant difference for either the kₐ or the k_{d} or both of the anti-CD3 binding domain to CD3ε T (epsilon/gamma) and CD3ε 6 (epsilon/delta) when measured under the same conditions, in particular when determined by SPR at 25° C. In certain embodiments thereof, the association rate constant kₐ and/or the dissociation rate constant k_{d} values that are within 1 fold of each other, 1.5 fold of each other, 2-fold of each other, 2.5-fold of each other or 3-fold of each other, i.e., association rate constant kₐ values of 1×10⁵ M⁻¹s⁻¹ and 3×10⁵ M⁻¹s⁻¹.

In certain embodiments, the first pMHC binding domain and/or the second pMHC binding domain binds the target pMHC complex with a binding affinity (K_{D}) of about 100 pM to about 20 nM (e.g., about 100 pM, about 150 pM, about 200 pM, about 250 pM, about 300 pM, about 350 pM, about 400 pM, about 450 pM, about 500 pM, about 550 pM, about 600 pM, about 650 pM, about 700 pM, about 750 pM, about 800 pM, about 850 pM, about 900 pM, about 950 pM, about 1 nM (1,000 pM), about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, or about 20 nM). In certain embodiments, the first pMHC binding domain and/or the second pMHC binding domain binds the target pMHC complex with a binding affinity (K_{D}) of about 100 pM to about 1 nM. In certain embodiments, the first pMHC binding domain and/or the second pMHC binding domain binds the target pMHC complex with a binding affinity (K_{D}) of about 100 pM to about 400 pM. In certain embodiments, the first pMHC binding domain and/or the second pMHC binding domain binds the target pMHC complex with a binding affinity (K_{D}) of about 500 pM to about 2 nM, or about 500 pM to about 3 nM, 500 pM to about 5 nM, or about 500 pM to about 10 nM, or about 100 pM to about 20 nM. In preferred embodiments, the said binding affinities are determined by SPR, as described elsewhere herein.

In some embodiments, the association rate constant kₐ of the pMHC binding domain is between about 1×10⁵ to about 1×10⁷ M⁻¹s⁻¹, preferably between about 0.5×10⁶ M⁻¹s⁻¹ to about 3×10⁶ M⁻¹s⁻¹, such as at least 0.5×10⁶ M⁻¹s⁻¹, at least 1×10⁶ M⁻¹s⁻¹, at least 2×10⁶ M⁻¹s⁻¹ or at least 3×10⁶ M⁻¹s⁻¹.

In some embodiments, the dissociation rate constant k_{d} of the pMHC binding domain is between about 1×10⁻¹ to about 1×10⁻⁶ s⁻¹, such as between about 1×10⁻² to about 1×10⁻⁵s⁻¹, such as at least 2×10⁻³ s⁻¹, at least 4×10⁻³ s⁻¹, at least 6×10⁻³ s⁻¹, at least 8×10⁻³ s⁻¹, at least 2×10⁴ s⁻¹, at least 4×10⁴ s⁻¹, at least 6×10⁻⁴ s⁻¹ or at least 8×10⁴ s⁻¹.

In certain embodiments, the antigen binding protein comprises a molecular weight of about 75 kDa to about 110 kDa (e.g., about 75 kDa, about 80 kDa, about 85 kDa, about 90 kDa, about 95 kDa, about 100 kDa, about 105 kDa or about 110 kDa). In certain embodiments, the antigen binding protein has increased serum half-life relative to an antigen binding protein with a molecular weight of less than about 60 kDa.

In certain embodiments, the antigen binding protein is a Fab-(scFv)₂ and comprises a single Fab domain which specifically binds to CD3, a first pMHC binding scFv linked to the C-terminus of the Fab domain heavy chain and a second pMHC binding scFv linked to the C-terminus of the Fab domain light chain, wherein both pMHC binding scFvs bind to the same target, such as a MAGE-A4 derived peptide presented on a HLA-A2 complex, and the variable heavy chain of both pMHC binding scFvs optionally or additionally comprises serine (S) at amino acid position 11, serine (S) at amino acid position 89, and serine (S) at amino acid position 108, according to Kabat numbering. Advantageously, the Fab domain comprises the first few amino acids of the antibody hinge region up to the first cysteine.

In certain embodiments, the antigen binding protein is a Fab-(scFv)₂ and comprises (i) a single Fab domain which specifically binds to CD3 with an affinity (K_{D}) from about 1 nM to about 50 nM, (ii) a first pMHC binding scFv linked to the C-terminus of the Fab domain heavy chain and (iii) a second pMHC binding scFv linked to the C-terminus of the Fab domain light chain, wherein both pMHC binding scFvs have a binding affinity (K_{D}) of about 500 pM to about 10 nM to the target pMHC complex.

An advantage of the antigen binding protein scaffolds of the disclosure is the intermediate molecular size of approximately 75-110 kDa. Blinatumomab, a bispecific T cell engager (BiTE), has shown excellent results in patients with relapsed or refractory acute lymphoblastic leukemia. Because of its small size (60 kDa), blinatumomab is characterized by a short serum half-life of several hours, and therefore continuous infusion is needed (see, U.S. Pat. No. 7,112,324 B1). The antigen binding proteins of the disclosure are expected to have significantly longer half-lives in comparison to smaller bispecific antibodies, such as BiTEs like blinatumomab, and thus, do not require continuous infusion due to their favorable half-life. An intermediate sized molecule may avoid kidney clearance and provide a half-life sufficient for improved tumor accumulation. While the antigen binding proteins of the disclosure have increased plasma half-life compared to other small bispecific formats, they still retain the tumor penetration ability. On the other hand, the molecules of the instant disclosure lacking an Fc domain are expected to have a shorter half-life than larger molecules including a Fc domain. A prolonged half-life may overstimulate T cells and lead to T cell exhaustion. Also, especially in solid tumors, a large molecular weight may translate into a lower degree of tumor penetration. In some embodiments, the in vivo half-life is of about 7 days.

The Fab domain of the antigen binding protein of the disclosure may serve as a specific heterodimerization scaffold to which the additional pMHC binding domains are linked. The natural and efficient heterodimerization properties of the heavy chain (Fd fragment) and light chain (L) of a Fab fragment makes the Fab fragment a useful scaffold. Additional binding domains may be in several different formats, including, but not limited to, another Fab domain, a scFv, or an sdAb.

Each chain of the Fab fragment can be extended at the N- or C-terminus with additional binding domains. The chains may be co-expressed in mammalian cells, where the host-cell Binding immunoglobulin protein (BiP) chaperone drives the formation of the heavy chain-light chain heterodimer (Fd:L). These heterodimers are stable, with each of the binders retaining their specific affinities. The two remaining pMHC binding domains may then be fused as scFvs or sdAbs to distinct Fab chains where each chain can be extended, e.g., at the C-terminus with an additional scFv or sdAb domain (see, for example, Schoonjans et al. J. Immunology, 165(12): 7050-7057, 2000; Schoonjans et al. Biomolecular Engineering, 17: 193-202, 2001.) An additional advantage of using Fabs as a heterodimerization unit is that Fab molecules are abundantly present in serum and therefore may be non-immunogenic when administered to a subject.

Based on, and in line with the overall disclosure content of the present specification, aspects and embodiments of the present invention include:

1. An antigen binding protein comprising:
a) a Fab domain which specifically binds to a cell surface protein of an immune cell, the Fab domain comprising a heavy chain and a light chain; and
at least a first peptide-MHC (pMHC) binding domain and at least a second pMHC binding domain, wherein both the at least first and the at least second pMHC binding domain are operably linked to the heavy chain of the Fab domain, and wherein the first pMHC binding domain binds to a first target pMHC complex and the second pMHC binding domain binds to a second target pMHC complex;
wherein the at least first and/or the at least second pMHC binding domain is each any one of a scFv, a scFab, a diabody, a sdAb (VHH) or a Fab or
b) a Fab domain which specifically binds to a cell surface protein of an immune cell, the Fab domain comprising a heavy chain and a light chain; and
at least a first peptide-MHC (pMHC) binding domain and at least a second pMHC binding domain, wherein both the at least first and the at least second pMHC binding domain are operably linked to the light chain of the Fab domain, and wherein the first pMHC binding domain binds to a first target pMHC complex and the second pMHC binding domain binds to a second target pMHC complex;
wherein the at least first and/or the at least second pMHC binding domain is each any one of a scFv, a scFab, a diabody, a sdAb (VHH) or a Fab.

2. The antigen binding protein of embodiment [1], wherein the antigen binding domain comprises at least 5 amino acids of an antibody hinge region, located at the C-terminus of the CH1 domain of the Fab domain which specifically binds to a cell surface protein of an immune cell.

3. The antigen binding protein of the [1] or [2], wherein the at least first target pMHC complex and the at least second target pMHC complex are the same or are different. Preferably, they are the same.

4. The antigen binding protein of any one of the above [1] to [3], wherein:
(i) the first pMHC binding domain is operably linked to the C-terminus of the heavy chain, and the second pMHC binding domain is operably linked to the N-terminus of the heavy chain; or
(ii) the first pMHC binding domain is operably linked to the N-terminus of the heavy chain, and the second pMHC binding domain is operably linked to the C-terminus of the heavy chain; or
(iii) the first pMHC binding domain is operably linked to the C-terminus of the light chain, and the second pMHC binding domain is operably linked to the N-terminus of the light chain; or
(ii) the first pMHC binding domain is operably linked to the N-terminus of the light chain, and the second pMHC binding domain is operably linked to the C-terminus of the light chain.

5. In any one of the above [1] to [4], the Fab domain which specifically binds to a cell surface protein of an immune cell comprises a variable heavy chain having a non-polar amino acid at position 11, 89 and/or 108, according to Kabat numbering, as described elsewhere herein.

6. In any one of the above [1] to [5], the at least first and/or the at least second pMHC binding domain a comprises a variable heavy chain having
(i) a polar amino acid as described elsewhere herein, such as serine, at position 11, 89 and/or 108, according to Kabat numbering, as described elsewhere herein; and/or
(ii) a deletion or substitution at position 112 and/or position 113, as described elsewhere herein.

7. The antigen binding protein of any one of the above [1] to [6], wherein the at least first and/or the at least second pMHC binding domain specifically targets an MHC restricted peptide derived of a tumor antigen or a viral antigen.

8. The antigen binding protein of any one of the above [1] to [7], wherein the cell surface protein of an immune cell is CD3, and wherein the immune cell is a T cell.

9. The antigen binding protein of any one of the above [1] to [8], wherein the Fab domain specifically binds to CD3 with a binding affinity (K_{D}) between about 1 nM to about 50 nM, as determined by SPR.

10. The antigen binding protein of any one of the above [1] to [9], wherein the at least first pMHC binding domain and/or the at least second pMHC binding domain binds the target peptide pMHC complex with a binding affinity (K_{D}) of about 100 pM to about 20 nM. In preferred embodiments, the at least first pMHC binding domain and/or the at least second pMHC binding domain binds the target peptide pMHC complex with a binding affinity (K_{D}) of about 500 pM to about 10 nM or of about 500 pM to about 5 nM.

11. The antigen binding protein of any one of the above [1] to [10], comprising a molecular weight of about 75 kDa to about 110 kDa.

12. The antigen binding protein of any one of the above [1] to [11], wherein the antigen binding protein has increased serum half-life relative to an antigen binding protein with a molecular weight of < about 60 kDa.

13. A composition comprising the antigen binding protein of any one of the above [1] to [12], preferably the composition is a pharmaceutical composition.

14. A method of treating cancer or a viral infection comprising the step of administering the antigen binding protein of any one of the above [1] to [12], or the composition of [13], to a patient in need thereof.

15. An antigen binding protein comprising:
a) a Fab domain which specifically binds CD3 on a T cell, the Fab domain comprising a heavy chain and a light chain;
b) at least a first peptide-MHC (pMHC) binding domain operably linked to the C-terminus of the heavy chain, wherein the first pMHC binding domain binds to a first target peptide-MHC complex; and
c) at least a second pMHC binding domain operably linked to the C-terminus of the light chain, wherein the second pMHC binding domain binds to a second target pMHC complex,
wherein the at least first and/or the at least second pMHC binding domain is each any one of a scFv, a scFab, a diabody, a sdAb (VHH), or a Fab.

16. An antigen binding protein comprising:
a) a Fab domain which specifically binds CD3 on a T cell, the Fab domain comprising a heavy chain and a light chain;
b) at least a first peptide-MHC (pMHC) binding domain operably linked to the C-terminus of the heavy chain, wherein the first pMHC binding domain binds to a first target peptide-MHC complex; and
c) at least a second pMHC binding domain operably linked to the N-terminus of the light chain, wherein the second pMHC binding domain binds to a second target pMHC complex,
wherein the at least first and/or the at least second pMHC binding domain is each any one of a scFv, a scFab, a diabody, a sdAb (VHH) or a Fab.

17. The antigen binding protein of [15] or [16], wherein the antigen binding domain comprises at least 5 amino acids, optionally at most 10 amino acids, of an antibody hinge region located at the C-terminus of the CH1 domain of the Fab domain.

18. The antigen binding protein of any one of the above [15] to [17], wherein the at least first target pMHC complex and the at least second target pMHC complex are the same or are different. Preferably, they are the same.

19. In any one of the above [15] to [18], the Fab domain comprises a variable heavy chain having a non-polar amino acid at position 11, 89 and/or 108, according to Kabat numbering, as described elsewhere herein.

20. In any one of the above [15] to [19], the at least first and/or the at least second pMHC binding domain comprises a variable heavy chain having
(i) a polar amino acid as described elsewhere herein, e.g. serine, at position 11, 89 and/or 108, according to Kabat numbering, as described elsewhere herein; and/or
(ii) a deletion or substitution at position 112 and/or position 113, as described elsewhere herein.

21. The antigen binding protein of any one of the above [15] to [20], wherein the at least first and/or the at least second pMHC binding domain specifically targets an MHC restricted peptide derived of a tumor antigen or a viral antigen.

22. The antigen binding protein of any one of the above [15] to [21], wherein the Fab domain specifically binds to CD3 with a binding affinity (K_{D}) between about 1 nM to about 50 nM, as determined by SPR.

23. The antigen binding protein of any one of the above [15] to [22], wherein the at least first pMHC binding domain and/or the at least second pMHC binding domain binds the target peptide pMHC complex with a binding affinity (K_{D}) of about 100 pM to about 20 nM. In preferred embodiments, the at least first pMHC binding domain and/or the at least second pMHC binding domain binds the target peptide pMHC complex with a binding affinity (K_{D}) of about 500 pM to about 10 nM or of about 500 pM to about 5 nM.

24. The antigen binding protein of any one of the above [15] to [23], comprising a molecular weight of about 75 kDa to about 110 kDa.

25. The antigen binding protein of any one of the above [15] to [24], wherein the antigen binding protein has increased serum half-life relative to an antigen binding protein with a molecular weight of less than about 60 kDa.

26. A composition comprising the antigen binding protein of any one of the above [15] to [25], preferably the composition is a pharmaceutical composition.

27. A method of treating cancer or a viral infection comprising the step of administering the antigen binding protein of any one of the above [15] to [25], or the composition of [26], to a patient in need thereof.

28. A bivalent bispecific antigen binding protein comprising:
a) a Fab domain which specifically binds to a cell surface protein of an immune cell; and
b) at least two peptide-MHC (pMHC) binding domains targeting the same pMHC complex, wherein
(i) one of the at least two pMHC binding domains is operably linked to the C-terminus of the heavy chain, and the other pMHC binding domain is operably linked to the C-terminus of the light chain, or
(ii) one of the at least two pMHC binding domains is operably linked to the C-terminus of the heavy chain, and the other pMHC binding domain is operably linked to the N-terminus of the light chain;
wherein the at least first and/or the at least second pMHC binding domain is each any one of a scFv, a scFab, a diabody, a sdAb (VHH) or a Fab; and
wherein said bivalent bispecific antigen binding protein:
triggers or provides for MHC-restricted T cell activation, as described elsewhere herein; and/or
induces immune cell-mediated cytotoxicity towards a cell comprising the pMHC complex with higher potency as compared to a corresponding monovalent bispecific antigen binding protein targeting a single pMHC complex, as determined under the same conditions. In some embodiments, said cell comprising the pMHC complex is a cancer cell. T cell activation may, e.g., be determined by IFN-7 (gamma) release, or may be determined by quantification of CD69 and CD25 markers on CD8+ T cell populations after 24 h using flow cytometry, as exemplified in the Examples.

29. The bivalent bispecific antigen binding protein of [28], wherein the at least first target pMHC complex and the at least second target pMHC complex are the same or are different. Preferably, they are the same.

30. The bivalent bispecific antigen binding protein of [28] or [29], comprising at least 5 amino acids of an antibody hinge region, located at the C-terminus of the CH1 domain of the Fab domain which specifically binds to a cell surface protein of an immune cell.

31. In any one of the above [28] to [30], the Fab domain which specifically binds to a cell surface protein of an immune cell comprises a variable heavy chain having a non-polar amino acid at position 11, 89 and/or 108, according to Kabat numbering, as described elsewhere herein.

32. In any one of the above [28] to [31], the at least two pMHC binding domains comprise a variable heavy chain having
(i) a polar amino acid as described elsewhere herein, e.g., serine, at position 11, 89 and/or 108, according to Kabat numbering, as described elsewhere herein; and/or
(ii) a deletion or substitution at position 112 and/or position 113, as described elsewhere herein.

33. The bivalent bispecific antigen binding protein of any of the above [28] to [32], wherein the at least two pMHC binding domains specifically target an MHC restricted peptide derived of a tumor antigen or a viral antigen.

34. The bivalent bispecific antigen binding protein of any one of the above [28] to [33], wherein the Fab domain specifically binds to CD3 with a binding affinity (K_{D}) between about 1 nM to about 50 nM, as determined by SPR.

35. The bivalent bispecific antigen binding protein of any one of the above [28] to [34], wherein the at least two pMHC binding domains bind the target peptide pMHC complex with a binding affinity (K_{D}) of about 100 pM to about 20 nM. In preferred embodiments, the at least first pMHC binding domain and/or the at least second pMHC binding domain binds the target peptide pMHC complex with a binding affinity (K_{D}) of about 500 pM to about 10 nM or of about 500 pM to about 5 nM.

36. The bivalent bispecific antigen binding protein of any one of the above [28] to [35], comprising a molecular weight of about 75 kDa to about 110 kDa.

37. The bivalent bispecific antigen binding protein of any one of the above [28] to [36], wherein the bivalent bispecific antigen binding protein has increased serum half-life relative to a bivalent bispecific antigen binding protein with a molecular weight of less than about 60 kDa.

38. A composition comprising the bivalent bispecific antigen binding protein of any one of the above [28] to [36], preferably the composition is a pharmaceutical composition.

39. A method of treating cancer or a viral infection comprising the step of administering the bivalent bispecific antigen binding protein of any one of the above [28] to [37], or the composition of [38], to a patient in need thereof.

40. A bispecific T cell engager which is bivalent for a pMHC target, wherein said bivalent T cell engager exhibits increased pMHC cell expressing toxicity than a corresponding monovalent bispecific.

41. The bispecific T cell engager of [40], having the structural and functional features as described elsewhere herein, e.g., in [1]-[14] or in [15]-[25].

42. A bispecific T cell engager, comprising a CD3 binding domain and at least one pMHC binding domain, preferably two pMHC complex binding domains, wherein the association rate constant kₐ and/or the dissociation rate constant k_{d} of the CD3 binding domain similar for both CD3-heterodimers CD3ε T (epsilon/gamma) and CD3ε 6 (epsilon/delta) when determined by SPR at 25° C.

43. The bispecific T cell engager of [42], having the structural and functional features as described elsewhere herein, e.g., in [1]-[14] or in [15]-[25].

44. The bispecific T cell engager of [42] or [43], wherein the association rate constant kₐ of the CD3 binding domain is between about 1×10⁵ to about 1×10⁷ M⁻¹s⁻¹ the dissociation rate constant k_{d} of the CD3 binding domain is between about 1×10⁻¹ to about 1×10⁻⁶ s⁻¹ the association rate constant, kₐ of the pMHC binding domain is between about 1×10⁵ to about 1×10⁷ M⁻¹s⁻¹ and the dissociation rate constant k_{d} of the pMHC binding domain is between about 1×10⁻¹ to about 1×10⁻⁶ s⁻¹.

45. The bispecific T cell engager of any of the above [44]-[42], wherein the association rate constant kₐ and/or the dissociation rate constant k_{d} of the CD3 binding domain are similar to the association rate constant kₐ and/or the dissociation rate constant k_{d} of the pMHC binding domains.

### Peptide-MHC Complex Binding Domains

As is known in the art, MHC molecules present peptides, in particular antigenic peptides, on the surface of cells to be recognized by immune cells. Accordingly, as will be appreciated by a skilled artisan, the term “pMHC complex” as used herein refers to a complex of an MHC molecule and a peptide, in particular an antigenic peptide, presented by the MHC molecule. This is commonly known as MHC-restricted antigen presentation. Accordingly, the peptide targeted by the pMHC binding domains is an MHC-restricted peptide. The peptide can thus be considered as target peptide or target antigenic peptide. Further, in accordance with the present disclosure, the terms “target pMHC binding domain” and “pMHC binding domain” may be used interchangeably herein, and in any case refer to the at least first and at least second pMHC binding domains referred to herein throughout. The terms “target peptide/antigen presented by a MHC molecule/complex” and “MHC restricted target peptide/antigen”, or similar expressions used throughout the present specification, may be used interchangeably herein.

While MHC occur in all vertebrates, the MHC in human is known as HLA (human leukocyte antigen). There are three classes of MHC molecules. The target peptide may be presented on a MHC class I complex (such as of serotype HLA-A, HLA-B, HLA-C, HLA-E, HLA-F, HLA-G, HLA-K or HLA-L, or their respective subtypes) or an MHC class II complex (such as the serotypes HLA-DP, HLA-DQ, HLA-DR, DM or DO, or their respective subtypes). Each of the serotypes comprise different subtypes. In one embodiment, the antigen binding protein targets a peptide bound to an HLA-A2-MHC complex, also termed HLA-A*02, in particular HLA-A*02:01.

In certain embodiments, the antigen binding protein selectively binds a pMHC complex of a given HLA subtype and a target peptide, but not to a pMHC complex of the same HLA subtype presenting a different peptide. In certain embodiments, the antigen binding protein selectively binds to a target peptide presented on a pMHC complex of a given HLA subtype, but not to the same of peptide presented on a pMHC complex of a different HLA subtype.

Accordingly, the present disclosure encompasses, in certain embodiments, a bispecific bivalent antigen binding protein, comprising a Fab domain which specifically binds to CD3; and no more than two pMHC binding domains, wherein both pMHC binding domains are targeting the same HLA-A complex (i.e., the antigen binding protein is bivalent with regard to the target pMHC complex), such as the same HLA-A2 complex, or are targeting the same peptide presented by a HLA-A complex, in particular presented by a HLA-A2 complex, wherein both pMHC binding domains are each a scFv, or are each a sdAb (VHH), and wherein (i) one of the two pMHC binding domains is operably linked to the C-terminus of the heavy chain of the CD3 binding domain, and the other pMHC binding domain is operably linked to the C-terminus of the light chain of the CD3 binding domain, or (ii) one of the two pMHC binding domains is operably linked to the C-terminus of the heavy chain of the CD3 binding domain, and the other pMHC binding domain is operably linked to the N-terminus of the light chain of the CD3 binding domain.

In certain embodiments, the MHC restricted peptide is derived from a tumor antigen or a viral antigen. In some embodiments, the MHC restricted peptide is a cancer testis antigen. In some embodiments, the MHC restricted peptide is a neoantigen. In certain embodiments, the MHC restricted peptide is derived from a NY-ESO-1 (New York esophageal squamous cell carcinoma-1) protein, PRAME (preferentially expressed antigen in melanoma) protein or SX-2 (Synovial Sarcoma, X breakpoint 2) protein.

In certain embodiments, the MHC restricted target peptide is derived from a MAGE protein, including the MAGE-A, -B, -C subfamily members. In some embodiments, the MHC restricted target peptide is derived from a MAGE-A protein, including but not limited to MAGE-A1, MAGE-A2, MAGE-A3, and MAGE-A4. In some embodiments, the MHC restricted target peptide is derived from a MAGE-A4 protein. As described elsewhere herein and as exemplified by the Examples and the drawings, in preferred embodiments, the target peptide is presented on an MHC class I molecule of serotype HLA-A, preferably HLA-A2, wherein the target peptide derived from a MAGE-A protein, preferably from a MAGE-A4 protein, thus certain antigen binding proteins described herein possess binding specificity to a MAGE-A4 peptide-MHC.

In one embodiment, the target peptide is GVYDGREHTV (SEQ ID NO.: 1) which corresponds to amino acids 230-239 of MAGE-A4. Thus, in one embodiment, a bivalent antigen binding protein of the present disclosure may have binding affinity (K_{D}) to the target peptide GVYDGREHTV (SEQ ID NO.: 1); and/or may trigger or provide for MHC-restricted T cell activation, as described elsewhere herein. T cell activation may, e.g., be determined by IFN-7 (gamma) release, or may be determined by quantification of CD69 and CD25 markers on CD8+ T cell populations after 24 h using flow cytometry, as exemplified in the Examples.

As described elsewhere herein and as exemplified in the Examples and the drawings, in some embodiments thereof, the at least first and at least second pMHC binding domain are the same and are in particular each an scFv and are the same, or are each a sdAb and are the same. Further, as described elsewhere herein and as exemplified in the Examples and the drawings, in some embodiments thereof, the target peptide is presented on an MHC class I molecule of serotype HLA-A2. In some embodiments thereof, the target (antigenic) peptide (or MHC restricted target peptide) is derived from a MAGE protein, preferably from a MAGE-A4 protein. As further described elsewhere herein and as further exemplified in the Examples and the drawings, in some embodiments, the antigen binding protein has no more than two pMHC binding domains, i.e., is limited with regard to pMHC binding domains to one first pMHC binding domain one second pMHC binding domain, which preferably are both an scFv and are the same, or which are both a sdAb and are the same.

Accordingly, in various embodiments, an antigen binding protein as provided by the present disclosure comprises at least a first pMHC binding domain and at least a second pMHC binding domain, each binding to a first or second pMHC complex presenting the target peptide GVYDGREHTV (SEQ ID NO.: 1). According to some embodiments described elsewhere herein, and as exemplified in the Examples and the drawings, an antigen binding protein as provided by the present disclosure is bivalent for the pMHC complex and comprises no more than two pMHC binding domains, wherein both pMHC binding domains binds to a pMHC complex presenting the target peptide GVYDGREHTV (SEQ ID NO.: 1). In some embodiments thereof, said bivalent antigen binding protein is bispecific, i.e., further to the binding specificity for the target peptide GVYDGREHTV (SEQ ID NO.: 1), it has binding specificity for a cell surface protein of an immune cell as described elsewhere herein, such CD3. Furthermore, in certain embodiments thereof, the pMHC complex presenting the target peptide of SEQ ID NO.: 1 is a pMHC class I complex, i.e., the target peptide is presented on an MHC class I molecule, particularly on an MHC class I molecule of serotype HLA-A (“HLA-A pMHC complex”), more particularly on an MHC class I molecule of serotype HLA-A2 (“HLA-A2 pMHC complex”), as described elsewhere herein. As described elsewhere herein, and as exemplified by the Examples and the drawings, in some embodiments thereof, both pMHC binding domains are each a scFv, or are each a sdAb (VHH). As described elsewhere herein, and as exemplified by the Examples and the drawings, in further embodiments thereof, (i) one of the two pMHC binding domains is operably linked to the C-terminus of the heavy chain of the CD3 binding domain, and the other pMHC binding domain is operably linked to the C-terminus of the light chain of the CD3 binding domain, or (ii) one of the two pMHC binding domains is operably linked to the C-terminus of the heavy chain of the CD3 binding domain, and the other pMHC binding domain is operably linked to the N-terminus of the light chain of the CD3 binding domain.

The present disclosure encompasses antigen binding proteins comprising scFvs and sdAbs as described in WO2022190009A1, which is hereby incorporated by reference. Accordingly, in various embodiments, an antigen binding protein as provided by the present disclosure comprises at least a first pMHC binding domain and at least a second pMHC binding domain, wherein at least one of the at least first and at least second pMHC binding domains comprises:
(a) a heavy chain variable (VH) domain comprising
(i) an HCDR1 amino acid sequence of SNYAMS (SEQ ID NO.: 26),
(ii) an HCDR2 amino acid sequence of IVSSGGTTYYAX₁X₂X₃KG (SEQ ID NO.: 27), wherein X₁ corresponds to amino acid S or D, X₂ corresponds to amino acid W or S, and X₃ corresponds to amino acid A or V, and
(iii) an HCDR3 amino acid sequence of DLYYGPX₄TX₅YX₆X₇X₈NL (SEQ ID NO.: 28), wherein X₄ corresponds to amino acid T, N, or S, X₅ corresponds to amino acid D or is absent, X₆ corresponds to amino acid S or F, X₇ corresponds to amino acid A or V, and X₈ corresponds to amino acid F or A; and
(b) a light chain variable (VL) domain comprising
(iv) an LCDR1 amino acid sequence of TADTLSRSYAS (SEQ ID NO.: 29),
(v) an LCDR2 amino acid sequence of RDTSRPS (SEQ ID NO.: 30), and
(vi) an LCDR3 amino acid sequence of ATX₉X₁₀X_{II}SGSNFQX₁₂ (SEQ ID NO.: 31), wherein X₉ corresponds to amino acid S or R, X₁₀ corresponds to amino acid D or P, X₁₁ corresponds to amino acid G, S, or F, and X₁₂ corresponds to amino acid L or A,
Wherein the antigen binding protein (in particular the at least first and/or second pMHC binding domain) has binding affinity (K_{D}) to a MHC complex presenting the target peptide GVYDGREHTV (SEQ ID NO.: 1); and/or
wherein the antigen binding protein triggers or provides for MHC-restricted T cell activation. T cell activation may, e.g., be determined by IFN-γ (gamma) release, or may be determined by quantification of CD69 and CD25 markers on CD8⁺ T cell populations after 24 h using flow cytometry, as exemplified in the Examples.

In certain embodiments of the disclosure, the antigen binding protein exhibits a specific binding affinity (K_{D}) to the MHC presented target peptide GVYDGREHTV (SEQ ID NO.: 1) in the low nanomolar and/or even picomolar range, as described elsewhere herein.

According to certain embodiments described elsewhere herein, and as exemplified in the Examples and the drawings, an antigen binding protein as provided by the present disclosure is bivalent for pMHC complex binding and comprises no more than two pMHC binding domains, wherein both pMHC binding domains comprise a VH and a VL domain as described above (i.e., comprising the CDRs of SEQ ID NOs: 26-31), wherein said antigen binding protein specifically binds to a MHC complex presenting GVYDGREHTV (SEQ ID NO.: 1), in particular HLA-A2 restricted GVYDGREHTV (SEQ ID NO.: 1), as described above; and/or wherein the antigen binding protein triggers or provides for MHC-restricted T cell activation as described above. In certain embodiments thereof, the bivalent antigen binding protein is bispecific and has binding specificity for a cell surface protein of an immune cell as described elsewhere herein, such as CD3. As described elsewhere herein, and as exemplified by the Examples and the drawings, in certain embodiments thereof, both said pMHC binding domains are each a scFv, or are each a sdAb (VHH). As described elsewhere herein, and as exemplified by the Examples and the drawings, in further embodiments thereof, (i) one of the two pMHC binding domains is operably linked to the C-terminus of the heavy chain of the CD3 binding domain, and the other pMHC binding domain is operably linked to the C-terminus of the light chain of the CD3 binding domain, or (ii) one of the two pMHC binding domains is operably linked to the C-terminus of the heavy chain of the CD3 binding domain, and the other pMHC binding domain is operably linked to the N-terminus of the light chain of the CD3 binding domain.

In one embodiment, the pMHC binding domain comprises a heavy chain variable (VH) domain comprising a HCDR1 sequence of SEQ ID NO.: 2, 8, 14 or 20.

In one embodiment, the pMHC binding domain comprises a heavy chain variable (VH) domain comprising a HCDR2 sequence of SEQ ID NO.: 3, 9, 15 or 21.

In one embodiment, the pMHC binding domain comprises a heavy chain variable (VH) domain comprising a HCDR3 sequence of SEQ ID NO.: 4, 10, 16 or 22.

In one embodiment, the pMHC binding domain comprises a light chain variable (VL) domain comprising a LCDR1 sequence of SEQ ID NO.: 5, 11, 17 or 23.

In one embodiment, the pMHC binding domain comprises a light chain variable (VL) domain comprising a LCDR2 sequence of SEQ ID NO.: 6, 12, 18 or 24.

In one embodiment, the pMHC binding domain comprises a light chain variable (VL) domain comprising a LCDR3 sequence of SEQ ID NO.: 7, 13, 19 or 25.

In one embodiment, the pMHC binding domain comprises a heavy chain variable (VH) domain comprising a HCDR1 sequence of SEQ ID NO.: 2, a HCDR2 sequence of SEQ ID NO.: 3, and a HCDR3 sequence of SEQ ID NO.: 4.

In one embodiment, the pMHC binding domain comprises a heavy chain variable (VH) domain comprising a HCDR1 sequence of SEQ ID NO.: 8, a HCDR2 sequence of SEQ ID NO.: 9, and a HCDR3 sequence of SEQ ID NO.: 10.

In one embodiment, the pMHC binding domain comprises a heavy chain variable (VH) domain comprising a HCDR1 sequence of SEQ ID NO.: 14, a HCDR2 sequence of SEQ ID NO.: 15, and a HCDR3 sequence of SEQ ID NO.: 16.

In one embodiment, the pMHC binding domain comprises a heavy chain variable (VH) domain comprising a HCDR1 sequence of SEQ ID NO.: 20, a HCDR2 sequence of SEQ ID NO.: 21, and a HCDR3 sequence of SEQ ID NO.: 22.

In one embodiment, the pMHC binding domain comprises a light chain variable (VL) domain comprising a LCDR1 sequence of SEQ ID NO.: 5, a LCDR2 sequence of SEQ ID NO.: 6, and a LCDR3 sequence of SEQ ID NO.: 7.

In one embodiment, the pMHC binding domain comprises a light chain variable (VL) domain comprising a LCDR1 sequence of SEQ ID NO.: 11, a LCDR2 sequence of SEQ ID NO.: 12, and a LCDR3 sequence of SEQ ID NO.: 13.

In one embodiment, the pMHC binding domain comprises a light chain variable (VL) domain comprising a LCDR1 sequence of SEQ ID NO.: 17, a LCDR2 sequence of SEQ ID NO.: 18, and a LCDR3 sequence of SEQ ID NO.: 19.

In one embodiment, the pMHC binding domain comprises a light chain variable (VL) domain comprising a LCDR1 sequence of SEQ ID NO.: 23, a LCDR2 sequence of SEQ ID NO.: 24, and a LCDR3 sequence of SEQ ID NO.: 25.

In one embodiment, the pMHC binding domain comprises (a) a heavy chain variable (VH) domain comprising a HCDR1 sequence of SEQ ID NO.: 2, a HCDR2 sequence of SEQ ID NO.: 3, and a HCDR3 sequence of SEQ ID NO.: 4 and (b) a light chain variable (VL) domain comprising a LCDR1 sequence of SEQ ID NO.: 5, a LCDR2 sequence of SEQ ID NO.: 6, and a LCDR3 sequence of SEQ ID NO.: 7.

In one embodiment, the pMHC binding domain comprises (a) a heavy chain variable (VH) domain comprising a HCDR1 sequence of SEQ ID NO.: 8, a HCDR2 sequence of SEQ ID NO.: 9, and a HCDR3 sequence of SEQ ID NO.: 10 and (b) a light chain variable (VL) domain comprising a LCDR1 sequence of SEQ ID NO.: 11, a LCDR2 sequence of SEQ ID NO.: 12, and a LCDR3 sequence of SEQ ID NO.: 13.

In one embodiment, the pMHC binding domain comprises (a) a heavy chain variable (VH) domain comprising a HCDR1 sequence of SEQ ID NO.: 14, a HCDR2 sequence of SEQ ID NO.: 15, and a HCDR3 sequence of SEQ ID NO.: 16 and (b) a light chain variable (VL) domain comprising a LCDR1 sequence of SEQ ID NO.: 17, a LCDR2 sequence of SEQ ID NO.: 18, and a LCDR3 sequence of SEQ ID NO.: 19.

In one embodiment, the pMHC binding domain comprises (a) a heavy chain variable (VH) domain comprising a HCDR1 sequence of SEQ ID NO.: 20, a HCDR2 sequence of SEQ ID NO.: 21, and a HCDR3 sequence of SEQ ID NO.: 22 and (b) a light chain variable (VL) domain comprising a LCDR1 sequence of SEQ ID NO.: 23, a LCDR2 sequence of SEQ ID NO.: 24, and a LCDR3 sequence of SEQ ID NO.: 25.

Accordingly, in various embodiments, an antigen binding protein as provided by the present disclosure comprises at least a first pMHC binding domain and at least a second pMHC binding domain, wherein at least one of the at least first and at least second pMHC binding domains comprises the CDR sequences of any one of (i) SEQ ID NOs: 2-7, (ii) SEQ ID NOs: 8-13, (iii) SEQ ID NOs: 14-19, or (iv) SEQ ID NOs: 20-25, wherein the antigen binding protein (in particular the at least first and/or second pMHC binding domain) has binding specificity to a MHC complex presenting the target peptide GVYDGREHTV (SEQ ID NO.: 1) as described above in the context of SEQ ID NOs: 26-31; and/or wherein the antigen binding protein triggers or provides for MHC-restricted T cell activation as described above in the context of SEQ ID NOs: 26-31.

According to various embodiments described elsewhere herein, and as exemplified in the Examples and the drawings, an antigen binding protein as provided by the present disclosure is bivalent for the pMHC complex and comprises no more than two pMHC binding domains, wherein both pMHC binding domains comprise a VH and a VL domain as described above (i.e., comprising the CDRs of any one of (i) SEQ ID NOs: 2-7, (ii) SEQ ID NOs: 8-13, (iii) SEQ ID NOs: 14-19, or (iv) SEQ ID NOs: 20-25), wherein the antigen binding protein (in particular the two pMHC binding domains) has binding affinity (K_{D}) to a MHC complex presenting the target peptide GVYDGREHTV (SEQ ID NO.: 1), in particular HLA-A2 restricted, as described above; and/or wherein the antigen binding protein triggers or provides for MHC-restricted immune cell activation as described above. In certain embodiments thereof, the bivalent antigen binding protein is bispecific and has binding specificity for CD3 as described elsewhere herein. More particularly, the immune cell binding domain of said antigen binding protein which specifically binds to CD3 is monovalent for CD3 and may be a Fab. As described elsewhere herein, and as exemplified by the Examples and the drawings, in some embodiments thereof, both pMHC binding domains are each a scFv, or are each a sdAb (VHH). As described elsewhere herein, and as exemplified by the Examples and the drawings, in further embodiments thereof, (i) one of the two pMHC binding domains is operably linked to the C-terminus of the heavy chain of the CD3 Fab domain, and the other pMHC binding domain is operably linked to the C-terminus of the light chain of the CD3 binding Fab domain, or (ii) one of the two pMHC binding domains is operably linked to the C-terminus of the heavy chain of the CD3 binding Fab domain, and the other pMHC binding domain is operably linked to the N-terminus of the light chain of the CD3 binding Fab domain.

In certain embodiments, the CDRs are derived from an antigen binding protein disclosed herein, such as those disclosed in Tables 2, 3 or 4.

TABLE 2
CDR sequences of exemplary pMHC binding domains targeting the
peptide of SEQ ID NO.: 1 presented by HLA-A*02:01

| | |
|---|---|
| SEQ ID NO.: 2 | SNYAMS |
| (M1 HCDR1) | |
| SEQ ID NO.: 3 | IVSSGGTTYYADSVKG |
| (M1 HCDR2) | |
| SEQ ID NO.: 4 | DLYYGPNTDYSAANL |
| (M1 HCDR3) | |
| SEQ ID NO.: 5 | TADTLSRSYAS |
| (M1 LCDR1) | |
| SEQ ID NO.: 6 | RDTSRPS |
| (M1 LCDR2) | |
| SEQ ID NO.: 7 | ATRPSSGSNFQA |
| (M1 LCDR3) | |
| SEQ ID NO.: 8 | SNYAMS |
| (M2 HCDR1) | |
| SEQ ID NO.: 9 | IVSSGGTTYYADSVKG |
| (M2 HCDR2) | |
| SEQ ID NO.: 10 | DLYYGPSTYFVANL |
| (M2 HCDR3) | |
| SEQ ID NO.: 11 | TADTLSRSYAS |
| (M2 LCDR1) | |
| SEQ ID NO.: 12 | RDTSRPS |
| (M2 LCDR2) | |
| SEQ ID NO.: 13 | ATRPSSGSNFQL |
| (M2 LCDR3) | |
| SEQ ID NO.: 14 | SNYAMS |
| (M3 HCDR1) | |
| SEQ ID NO.: 15 | IVSSGGTTYYASWAKG |
| (M3 HCDR2) | |
| SEQ ID NO.: 16 | DLYYGPTTYSAANL |
| (M3 HCDR3) | |
| SEQ ID NO.: 17 | TADTLSRSYAS |
| (M3 LCDR1) | |
| SEQ ID NO.: 18 | RDTSRPS |
| (M3 LCDR2) | |
| SEQ ID NO.: 19 | ATRDFSGSNFQL |
| (M3 LCDR3) | |
| SEQ ID NO.: 20 | SNYAMS |
| (M4 HCDR1) | |
| SEQ ID NO.: 21 | IVSSGGTTYYASWAKG |
| (M4 HCDR2) | |
| SEQ ID NO.: 22 | DLYYGPTTYSAFNL |
| (M4 HCDR3) | |
| SEQ ID NO.: 23 | TADTLSRSYAS |
| (M4 LCDR1) | |

| | |
|---|---|
| SEQ ID NO.: 24 | RDTSRPS |
| (M4 LCDR2) | |
| SEQ ID NO.: 25 | ATRPSSGSNFQA |
| (M4 LCDR3) | |
| SEQ ID NO.: 26 | SNYAMS |
| Consensus | |
| HCDR1 | |
| SEQ ID NO.: 27 | IVSSGGTTYYAX₁X₂X₃KG |
| Consensus | wherein X₁ corresponds to amino acid S or D, X₂ corresponds to |
| HCDR2 | amino acid W or S, and X₃ corresponds to amino acid A or V |
| SEQ ID NO.: 28 | DLYYGPX₄TX₅YX₆X₇X₈NL |
| Consensus | wherein X₄ corresponds to amino acid T, N, or S, X₅ corresponds |
| HCDR3 | to amino acid D or is absent, X₆ corresponds to amino acid S or F, |
| | X₇ corresponds to amino acid A or V, and X₈ corresponds to amino |
| | acid F or A |
| SEQ ID NO.: 29 | TADTLSRSYAS |
| Consensus | |
| LCDR1 | |
| SEQ ID NO.: 30 | RDTSRPS |
| Consensus | |
| LCDR2 | |
| SEQ ID NO.: 31 | ATX₉X₁₀X₁₁SGSNFQX₁₂ |
| Consensus | wherein X₉ corresponds to amino acid S or R, X₁₀ corresponds to |
| LCDR3 | amino acid D or P, X₁₁ corresponds to amino acid G, S, or F, and |
| | X₁₂ corresponds to amino acid L or A. |

In one embodiment, the antigen binding protein comprises an amino acid sequence of SEQ ID NOs: 32, 34, 36 or 38. In one embodiment, the antigen binding protein comprises an amino acid sequence of SEQ ID NOs: 33, 35, 37 or 39. In one embodiment, the antigen binding protein comprises the amino acid sequences of SEQ ID NOs: 32 and 33. In one embodiment, the antigen binding protein comprises the amino acid sequences of SEQ ID NOs: 34 and 35. In one embodiment, the antigen binding protein comprises the amino acid sequences of SEQ ID NOs: 36 and 37. In one embodiment, the antigen binding protein comprises the amino acid sequences of SEQ ID NOs: 38 and 39.

Also encompassed are variants of the sequences disclosed herein. A variant amino acid or nucleic acid sequence differs from its parental sequence by virtue of insertion (including addition), deletion and/or substitution of one or more amino acid residues or nucleobases, respectively, while retaining at least one desired activity of the parent sequence disclosed herein, e.g., specific antigen binding. Variants may be artificially engineered or naturally occurring, such as e.g., allelic or splice variants.

Thus, in certain embodiments, a variant antigen binding protein retains specific binding to its target (e.g., an HLA-A2 restricted GVYDGREHTV, SEQ ID NO.: 1) and/or competes with an antigen binding protein disclosed herein for binding to its target. In certain embodiments, the variant antigen binding protein comprises an amino acid sequence being at least about 80%, 85%, 90%, 95%, 96%, 97%, 98%, 99% or 100% identical to an amino acid sequence disclosed herein. In certain embodiments, the variant antigen binding protein comprises 1, 2, 3, 4, 5, 6, 7, 8, 9, or 10 substitutions with regard to the parental amino acid sequence.

TABLE 3
Heavy and light chain amino acid sequences of exemplary pMHC
domains. CDR sequences are highlighted in bold underlined text.

| | |
|---|---|
| SEQ ID NO.: 32 | EVQLLESGGGSVQPGGSLRLSCTVSGFSLSNYAMSWVRQ |
| | APGKGLEWIGIVSSGGTTYYADSVKGRFTISRDNSKNTVY |
| | LQMNSLRAEDTASYYCAKDLYYGPNTDYSAANLWGQGT |
| | SVTVSS |
| SEQ ID NO.: 33 | QSVLTQDPAVSVALGQTVRITCTADTLSRSYASWYQQKP |
| | GQAPVLVIYRDTSRPSGIPDRFSGSSSGNTASLTITGAQAE |
| | DEADYYCATRPSSGSNFQAFGGGTKLTVLG |
| SEQ ID NO.: 34 | EVQLLESGGGSVQPGGSLRLSCTVSGFSLSNYAMSWVRQ |
| | APGKCLEWIGIVSSGGTTYYADSVKGRFTISRDNSKNTVY |
| | LQMNSLRAEDTASYYCAKDLYYGPSTYFVANLWGQGTS |
| | VTVSS |
| SEQ ID NO.: 35 | QSVLTQDPAVSVALGQTVRITCTADTLSRSYASWYQQKP |
| | GQAPVLVIYRDTSRPSGIPDRFSGSSSGNTASLTITGAQAE |
| | DEADYYCATRPSSGSNFQLFGCGTKLTVLG |
| SEQ ID NO.: 36 | EVQLLESGGGSVQPGGSLRLSCTVSGFSLSNYAMSWVRQ |
| | APGKCLEWIGIVSSGGTTYYASWAKGRFTISKDTSKNTV |
| | YLQMNSLRAEDTASYYCAKDLYYGPTTYSAANLWGQGT |
| | SVTVSS |
| SEQ ID NO.: 37 | SYELTQPPSVSVSPGQTASITCTADTLSRSYASWYQQKPG |
| | QSPVLVIYRDTSRPSGIPERFSGSNSGNTATLTISGTQAMD |
| | EADYYCATRDESGSNFQLFGCGTKLTVLG |
| SEQ ID NO.: 38 | EVQLLESGGGSVQPGGSLRLSCTVSGFSLSNYAMSWVRQ |
| | APGKGLEYIGIVSSGGTTYYASWAKGRFTISRDNSKNTVY |
| | LQMNSLRAEDTASYYCAKDLYYGPTTYSAFNLWGQGTS |
| | VTVSS |
| SEQ ID NO.: 39 | SYELTQPPSVSVSPGQTASITCTADTLSRSYASWYQQKPG |
| | QSPVLVIYRDTSRPSGIPERFSGSNSGNTATLTISGTQAMD |
| | EADYYCATRPSSGSNFQAFGGGTKLTVLG |

Accordingly, in various embodiments, an antigen binding protein as provided by the present disclosure comprises at least a first pMHC binding domain and at least a second pMHC binding domain, wherein at least one of the at least first and at least second pMHC binding domains comprises the VH/VL sequences of any one of (i) SEQ ID NOs: 32-33, (ii) SEQ ID NOs: 34-35, (iii) SEQ ID NOs: 36-37, or (iv) SEQ ID NOs: 38-39, wherein the antigen binding protein (in particular the at least first and/or second pMHC binding domain) has binding affinity (K_{D}) to the target peptide GVYDGREHTV (SEQ ID NO.: 1) as described above in the context of SEQ ID NOs: 26-31; and/or wherein the antigen binding protein triggers or provides for MHC-restricted T cell activation as described above in the context of SEQ ID NOs: 26-31.

According to preferred embodiments described elsewhere herein, and as exemplified in the Examples and the drawings, an antigen binding protein as provided by the present disclosure is bivalent for the pMHC complex and comprises no more than two pMHC binding domains, wherein both pMHC binding domains comprise a VH and a VL domain as described above (i.e., comprising the VH/VL sequences of any one of (i) SEQ ID NOs: 32-33, (ii) SEQ ID NOs: 34-35, (iii) SEQ ID NOs: 36-37, or (iv) SEQ ID NOs: 38-39), wherein the antigen binding protein (in particular the two pMHC binding domains) specifically binds to the target pMHC presenting GVYDGREHTV (SEQ ID NO.: 1), in particular HLA-A2 restricted, as described above; and/or wherein the antigen binding protein triggers or provides for MHC-restricted T cell activation as described above. In preferred embodiments, the bivalent antigen binding protein is bispecific and has binding specificity for a cell surface protein of an immune cell as described elsewhere herein, such as binding specificity for CD3 as described elsewhere herein. In some embodiments thereof, the immune cell binding domain is a Fab domain which specifically binds to CD3. As described elsewhere herein, and as exemplified by the Examples and the drawings, in some embodiments thereof, both pMHC binding domains are each a scFv, or are each a sdAb (VHH). As described elsewhere herein, and as exemplified by the Examples and the drawings, in further embodiments thereof, (i) one of the two pMHC binding domains is operably linked to the C-terminus of the heavy chain of the CD3 binding Fab domain, and the other pMHC binding domain is operably linked to the C-terminus of the light chain of the CD3 binding Fab domain, or (ii) one of the two pMHC binding domains is operably linked to the C-terminus of the heavy chain of the CD3 binding (Fab) binding domain, and the other pMHC binding domain is operably linked to the N-terminus of the light chain of the CD3 binding Fab domain.

Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 79, 81 and 82 or variants thereof; and no more than two pMHC binding domains targeting the same pMHC complex, wherein both pMHC binding domains are each a scFv, said scFvs comprising the CDRs of SEQ ID NOs: 26-31, the bispecific bivalent antigen binding protein e.g., being a Fab-(scFv)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 80, 81 and 82 or variants thereof; and no more than two pMHC binding domains targeting the same pMHC complex, wherein both pMHC binding domains are each a scFv, said scFvs comprising the CDRs of SEQ ID NOs: 26-31, the bispecific bivalent antigen binding protein e.g., being a Fab-(scFv)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 79, 81 and 82 or variants thereof; and no more than two pMHC binding domains targeting the same pMHC complex, wherein both pMHC binding domains are each a scFv, said scFvs comprising the CDRs of (i) SEQ ID NOs: 2-7, (ii) SEQ ID NOs: 8-13, (iii) SEQ ID NOs: 14-19, or (iv) SEQ ID NOs: 20-25, or variants thereof, respectively, the bispecific bivalent antigen binding protein e.g., being a Fab-(scFv)₂. Accordingly, the present disclosure encompasses, in certain embodiments, a bispecific bivalent antigen binding protein, comprising an anti-CD3-binding domain comprising the HCDR sequences of SEQ ID NOs.: 76, 77 and 78 and the LCDR sequences of SEQ ID NOs.: 80, 81 and 82 or variants thereof; and no more than two pMHC binding domains targeting the same pMHC complex, wherein both pMHC binding domains are each a scFv, said scFvs comprising the CDRs of (i) SEQ ID NOs: 2-7, (ii) SEQ ID NOs: 8-13, (iii) SEQ ID NOs: 14-19, or (iv) SEQ ID NOs: 20-25, or variants thereof, respectively, the bispecific bivalent antigen binding protein e.g., being a Fab-(scFv)₂. Accordingly, the present disclosure encompasses, in certain embodiments, a bispecific bivalent antigen binding protein, comprising an anti-CD3-binding domain comprising the VL sequence of SEQ ID NO.: 83 and the VH sequence of SEQ ID NO.: 84 or variants thereof; and no more than two pMHC binding domains targeting the same pMHC complex, wherein both pMHC binding domains are each a scFv, said scFvs comprising the VH/VL sequences of any one of (i) SEQ ID NOs: 32-33, (ii) SEQ ID NOs: 34-35, (iii) SEQ ID NOs: 36-37, or (iv) SEQ ID NOs: 38-39, or variants thereof, respectively, the bispecific bivalent antigen binding protein e.g., being a Fab-(scFv)₂. The exemplary pMHC binding domains targeting the peptide of SEQ ID NO.: 1 presented by HLA-A*02:01 and sequences recited above are described in further detail in U.S. 20220380472A1 and U.S. Provisional Patent Application Ser. No. 63/318,163, filed Mar. 9, 2022, the contents of each are incorporated herein by reference.

### Reduction of Anti-Drug Antibody Binding

Anti-drug antibodies (ADAs) may affect the risk profile and efficacy of a biological drug. If neutralizing, they may block the drug's ability to bind to its target. It is therefore a regulatory requirement to test biologic drugs for the binding of anti-drug antibodies and their neutralizing potential. Anti-drug antibody assays are e.g., detailed in WO2007101661A1 (Hoffmann La Roche), WO2018178307A1 (Ablynx), WO2021046316A2 (Adverum Biotechnologies, Charles River), and US20180088140A1 (Genzyme Corporation), each of which is incorporated herein by reference.

Anti-drug antibodies binding to a tumor targeting domain of an antigen binding protein may lead to clustering of said antigen binding protein when each variable domain of the ADA binds to one tumor targeting domain of two antigen binding proteins. The two or more CD3 binding domains on said antigen binding protein cluster and overstimulate the targeted T cell in the absence of target engagement, thereby leading to off-target toxicity. Unspecific stimulation of the T-cells may lead to systemic cytokine release.

Generally, there is a need in the art to develop safer and more effective bispecific antibodies for cancer immunotherapy.

The inventors have found that certain mutations in the tumor antigen binding domain of a T cell engager reduce ADA response and at the same time reduce nonspecific T cell stimulation in the absence of target engagement. Thereby, a highly effective and safe approach for cancer immunotherapy is provided.

In one aspect, the disclosure provides a method of reducing nonspecific T cell activation of a T cell engaging multispecific antigen binding protein, wherein the multispecific antigen binding protein comprises a first binding domain specifically targeting CD3 and a second binding domain specifically targeting a tumor antigen, wherein the multispecific antigen binding protein comprises at least one variable heavy chain, the method comprising the steps of: a) substituting a variable heavy chain amino acid at position 11, 89, and/or 108, according to Kabat numbering, with a polar amino acid; and b) deleting a serine (S) at position 113, according to Kabat numbering.

In certain embodiments, the polar amino acid of step a) is serine (S) and/or threonine (T).

In certain embodiments, the heavy chain amino acid is substituted with serine (S) at heavy chain amino acid position 11, serine (S) or threonine (T) at heavy chain amino acid position 89, and/or serine (S) or threonine (T) at heavy chain amino acid position 108, according to Kabat numbering.

In certain embodiments, the heavy chain amino acid is substituted with serine (S) at heavy chain amino acid position 11, serine (S) at heavy chain amino acid position 89, and serine (S) at heavy chain amino acid position 108, according to Kabat numbering.

In certain embodiments, step b) further comprises the step of deleting a serine (S) at position 112, according to Kabat numbering.

In certain embodiments, the method further comprises adding alanine (A), glycine (G) or threonine (T) at Kabat amino position 112 or 113.

In certain embodiments, the method further comprises adding alanine (A) at Kabat amino position 112 or 113.

In certain embodiments, the multispecific antigen binding protein is monovalent, bivalent or multivalent.

In certain embodiments, the antigen binding protein of said method is a Fab-sdAb, Fab-(sdAb)₂, a Fab-scFv or a Fab-(scFv)₂, F(ab′)₂fragment, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomAB, quadroma, CrossMab, DuoBody, triabody and tetrabody, or MATCH.

In certain embodiments, the second binding domain specifically targets a pMHC.

In certain embodiments, the multispecific antigen binding protein further comprises a third binding domain specifically targeting a pMHC.

In certain embodiments, the second binding domain and the third binding domain specifically target the same pMHC or different pMHC.

In certain embodiments, the antigen binding protein comprises one binding domain specifically targeting CD3 and one binding domain specifically targeting a pMHC.

In certain embodiments, the antigen binding protein comprises one binding domain specifically targeting CD3 and two binding domains specifically targeting a pMHC.

In certain embodiments, the two binding domains specifically targeting a pMHC are the same.

In certain embodiments, the pMHC binding domain specifically targets a MHC restricted peptide derived of a tumor antigen or a viral antigen.

In certain embodiments, the binding affinity (K_{D}) for CD3 is between about 1 nM to about 50 nM, optionally between about 20 nM to 50 nM, as determined by SPR.

In certain embodiments, the binding affinity (K_{D}) for CD3 is of about 1 nM, of about 10 nM, or of about 50 nM, as determined by SPR.

In certain embodiments, the binding affinity (K_{D}) for CD3 is of about 1 nM, of about 10 nM, or of about 50 nM, as determined by SPR.

In certain embodiments, the binding affinity (K_{D}) for the pMHC is of about 100 pM to about 20 nM (e.g., about 100 pM, about 150 pM, about 200 pM, about 250 pM, about 300 pM, about 350 pM, about 400 pM, about 450 pM, about 500 pM, about 550 pM, about 600 pM, about 650 pM, about 700 pM, about 750 pM, about 800 pM, about 850 pM, about 900 pM, about 950 pM, about 1 nM (1,000 pM), about 2 nM, about 3 nM, about 4 nM, or about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, or about 20 nM). In certain embodiments, the binding affinity (K_{D}) for the pMHC is of about 100 pM to about 10 nM. In certain embodiments, the binding affinity (K_{D}) for the pMHC is of about 500 pM to about 10 nM. In certain embodiments, the binding affinity (K_{D}) for the pMHC is of about 500 pM to about 5 nM. In certain embodiments, the binding affinity (K_{D}) for the pMHC is of about 500 pM to about 2 nM. In certain embodiments, the binding affinity (K_{D}) for the pMHC is 500 pM to about 1 nM,

In another aspect, the disclosure provides a multispecific antigen binding protein obtainable by the methods described above.

In another aspect, the disclosure provides an antigen binding protein comprising at least one first binding domain specific for CD3 and at least one second binding domain specific for a tumor antigen, each binding domain comprising at least one variable heavy chain, wherein at least one variable heavy chain comprises a polar amino acid at position 11, 89 and/or 108, according to Kabat numbering.

In certain embodiments, the variable heavy chain is of said second binding domain.

In certain embodiments, the polar amino acid is serine (S) and/or threonine (T).

In certain embodiments, the variable heavy chain comprises serine (S) at heavy chain amino acid position 11, serine (S) or threonine (T) at heavy chain amino acid position 89, and serine (S) or threonine (T) at heavy chain amino acid position 108, according to Kabat numbering.

In certain embodiments, the variable heavy chain comprises serine (S) at heavy chain amino acid position 11, serine (S) at heavy chain amino acid position 89, and serine (S) at heavy chain amino acid position 108, according to Kabat numbering.

In certain embodiments, the variable heavy chain has a serine (S) at position 113 deleted, according to Kabat numbering.

In certain embodiments, the variable heavy chain has serine (S) at position 112 and 113 deleted, according to Kabat numbering.

In certain embodiments, the antigen binding protein comprises alanine (A), glycine (G) or threonine (T) at position 112, according to Kabat numbering, in particular alanine (A).

In certain embodiments, the tumor antigen is a pMHC.

In certain embodiments, the pMHC binding domain specifically targets a MHC restricted peptide derived of a tumor antigen or a viral antigen.

In certain embodiments, the antigen binding protein has an affinity (K_{D}) for CD3 of about 1 nM to about 50 nM, optionally between about 20 nM to 50 nM, as determined by SPR.

In certain embodiments, the antigen binding protein has an affinity (K_{D}) for CD3 of about 1 nM, of about 10 nM or of about 50 nM, as determined by SPR.

In certain embodiments, the first binding domain specific for CD3 is a Fab fragment.

In certain embodiments, the antigen binding protein comprises two or more pMHC binding domains.

In certain embodiments, the pMHC binding domain is a scFv or an sdAb.

In certain embodiments, the antigen binding protein has an affinity (K_{D}) for the pMHC of about 100 pM to about 20 nM (e.g., about 100 pM, about 150 pM, about 200 pM, about 250 pM, about 300 pM, about 350 pM, about 400 pM, about 450 pM, about 500 pM, about 550 pM, about 600 pM, about 650 pM, about 700 pM, about 750 pM, about 800 pM, about 850 pM, about 900 pM, about 950 pM, about 1 nM (1,000 pM), about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, or about 20 nM). In certain embodiments, the antigen binding protein has an affinity (K_{D}) for the pMHC of about 100 pM to about 1 nM. In certain embodiments, the antigen binding protein has an affinity (K_{D}) for the pMHC of about 500 pM to about 2 nM. In certain embodiments, the antigen binding protein has an affinity (KD) for the pMHC of about 500 pM to about 3 nM. In certain embodiments, the antigen binding protein has an affinity (KD) for the pMHC of about 500 pM to about 5 nM.

In certain embodiments, said antigen binding protein is a Fab-sdAb, Fab-(sdAb)₂, a Fab-scFv or a Fab-(scFv)₂, F(ab′)₂fragment, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomAB, quadroma, CrossMab, DuoBody, triabody and tetrabody, or MATCH.

### Expression of Antigen Binding Proteins

In one aspect, polynucleotides or nucleic acids encoding the antigen binding proteins disclosed herein are provided. Methods of making a antigen binding protein comprising expressing these polynucleotides or nucleic acids are also provided.

Polynucleotides encoding the antigen binding proteins disclosed herein are typically inserted in an expression vector for introduction into host cells that may be used to produce the desired quantity of the antigen binding proteins. Accordingly, in certain aspects, the invention provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the antigen binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antigen binding proteins featured in the invention can also be expressed in non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

### Engineering and Optimization of Antigen Binding Proteins

The antigen binding proteins of the disclosure may be engineered or optimized. As used herein, “optimized” or “optimization” refers to the alteration of an antigen binding protein to improve one or more functional properties. Alteration includes, but is not limited to, deletions, substitutions, additions, and/or modifications of one or more amino acids within an antigen binding protein.

As used herein, the term “functional property” is a property of an antigen binding protein for which an improvement (e.g., relative to a conventional antigen binding protein, such as an antibody) is desirable and/or advantageous to one of skill in the art, e.g., in order to improve the manufacturing properties or therapeutic efficacy of an antigen binding protein. In one embodiment, the functional property is stability (e.g., thermal stability). In another embodiment, the functional property is solubility (e.g., under cellular conditions). In yet another embodiment, the functional property is aggregation behavior. In still another embodiment, the functional property is protein expression (e.g., in a prokaryotic cell). In yet another embodiment the functional property is refolding behavior following inclusion body solubilization in a manufacturing process. In certain embodiments, the functional property is not an improvement in antigen binding affinity. In another embodiment, the improvement of one or more functional properties has no substantial effect on the binding affinity of the antigen binding protein.

In certain embodiments, the antigen binding protein of the disclosure is an scFv and is optimized by identifying preferred amino acid residues to be substituted, deleted, and/or added at amino acid positions of interest (e.g., amino acid positions identified by comparing a database of scFv sequences having at least one desirable property, e.g., as selected with Quality Control (QC) assay, versus a database of mature antibody sequences, e.g., the Kabat database) in an antigen binding protein. Thus, the disclosure further provides “enrichment/exclusion” methods for selecting a particular amino acid residue. Still further, the disclosure provides methods of engineering antigen binding proteins (e.g., scFvs) by mutating particular framework amino acid positions identified using the “functional consensus” approach described herein. In certain embodiments, the framework amino acid positions are mutated by substituting the existing amino acid residue by a residue which is found to be an “enriched” residue using the “enrichment/exclusion” analysis methods described herein. In one aspect, the disclosure provides a method of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database. ScFv optimization is described in further detail in WO2008110348, WO2009000099, WO2009000098, and WO2009155725, all of which are incorporated herein by reference.

In those aspects of the disclosure where the presence of an Fc domain is practicable, the antigen binding protein may comprise an Fc domain which is modified such that it does not induce cytotoxic immune responses and/or does not activate complement. For example, one or more substitutions may be introduced into the Fc domain so that its ADCC/ADCP or CDC effector function is inactivated. Such antigen binding protein has the advantage of increased half-life when compared to antibody fragments with a molecular weight below 60 kDa, without mediating mediate cytotoxic immune responses.

Chemical and/or Biological Modifications

In one aspect, the antigen binding protein is chemically and/or biologically modified. For example, the antigen binding protein may be glycosylated, phosphorylated, hydroxylated, PEGylated, HESylated, PASylated, sulfated, labeled with dyes and/or radioisotopes, conjugated with enzymes and/or toxins, and/or Albumin binding or fusion technology. Likewise, any nucleic acid sequence, plasmid or vector and/or host cell described herein may be modified accordingly.

Such modification may for example be done to optimize pharmacokinetics, the water solubility or to lower side effects. For example, PEGylation, PASylation, HESylation and/or the fusion to serum albumin may be applied to slow down renal clearance, thereby increasing plasma half-life time of the antigen binding protein. In ne embodiment, the antigen binding molecules of the disclosure are operably linked to human serum albumin. In one embodiment, a modification adds a different functionality to the antigen binding protein, for example, a detection label for diagnostics or a toxin to combat cancer cells even more efficiently.

In one embodiment, the antigen binding protein is glycosylated. Glycosylation refers to a process that attaches carbohydrates to proteins. In biological systems, this process is performed enzymatically within the cell as a form of co-translational and/or post-translational modification. A protein can also be chemically glycosylated. The carbohydrates may be N-linked to a nitrogen of asparagine or arginine side-chains; O-linked to the hydroxy oxygen of serine, threonine, tyrosine, hydroxylysine, or hydroxyproline side-chains; employ xylose, fucose, mannose, and N-acetylglucosamine attached to a phospho-serine; and/or adding mannose sugar to a tryptophan residue found in a specific recognition sequence. Glycosylation patterns may, e.g., be controlled by choosing appropriate cell lines, culturing media, protein engineering manufacturing modes and process strategies (see., HOSSLER, P. Optimal and consistent protein glycosylation in mammalian cell culture. Glycobiology 2009, vol. 19, no. 9, p. 936-949.). In some embodiments, the glycosylation patterns of the antigen binding proteins described herein are modified to enhance ADCC and CDC effector function.

The antigen binding protein may be engineered to control or alter the glycosylation pattern, e.g., by deleting and/or adding of one or more glycosylation sites. The creation of glycosylation sites can e.g., be accomplished by introducing the corresponding enzymatic recognition sequence into the amino acid sequence of the antigen binding protein.

In some embodiments, the antigen binding protein is PEGylated. PEGylation may alter the pharmacodynamic and pharmacokinetic properties of a protein. Additionally, PEGylation may reduce the immunogenicity by shielding the PEGylated antigen binding protein from the immune system and/or alter its pharmacokinetics by, e.g., increasing the in vivo stability of the antigen binding protein, protecting it from proteolytic degradation, extending its half-life time and by altering its biodistribution. Typically, polyethylene-glycol (PEG) of an appropriate molecular weight is covalently attached to the protein. Similar effects may be achieved using PEG mimetics, e.g., HESylating, PASylating, or XTENylating the antigen binding protein. HESylation utilizes hydroxyethyl starch (“HES”) derivatives. During PASylation, the antigen binding protein is linked to conformationally disordered polypeptide sequences composed of the amino acids proline (P), alanine (A) and serine (S), and XTENylation employs a similar, intrinsically disordered XTEN-polypeptide

In certain embodiments, the antigen binding protein is labelled with or conjugated to a second moiety which attributes one or more ancillary functions to the antigen binding protein. For example, the second moiety may have an additional immunological effector function, be effective in drug targeting or useful for detection. The second moiety can, e.g., be chemically linked or fused genetically to the antigen binding protein using known methods in the art. As used herein, the term “label” refers to any substance or ion which is indicative of the presence of the antigen binding protein when detected or measured by physical or chemical means, either directly or indirectly. For example, the label may be directly detectable by, without being limited to, light absorbance, fluorescence, reflectivity, light scatter, phosphorescence, or luminescence properties, molecules or ions detectable by their radioactive properties or molecules or ions detectable by their nuclear magnetic resonance or paramagnetic properties. Examples of indirect detection include light absorbance or fluorescence; for example, various enzymes which cause appropriate substrates to convert, e.g., from non-light absorbing to light absorbing molecules, or from non-fluorescent to fluorescent molecules. A labelled antigen binding protein is particularly useful for in vitro and in vivo detection or diagnostic purposes. For example, an antigen binding protein labelled with a suitable radioisotope, enzyme, fluorophore or chromophore can be detected by radioimmunoassay (RIA), enzyme-linked immunosorbent assay (ELISA), or flow cytometry-based single cell analysis (e.g., FACS analysis), respectively. Similarly, the nucleic acids and/or vectors disclosed herein can be labeled for detection or diagnostic purposes, e.g., using labelled fragments thereof as probes in hybridization assays.

Non-limiting examples of second moieties include radioisotopes (35S, 32P, 14C, 18F, and/or 125I), apoenzymes, enzymes (e.g., alkaline phosphatase, horseradish peroxidase, beta-galactosidase and/or angiogenin), co-factors, peptide moieties (e.g., a HIS-tag), proteins (e.g. lectin, serum albumin), carbohydrates (e.g., mannose-6-phosphate tags), fluorophores (e.g., fluorescein isothiocyanate (FITC)), phycoerythrin, green/blue/red or other fluorescent proteins, allophycocyanin (APC), chromophores, vitamins (e.g., biotin), chelators, antimetabolites (e.g., methotrexate), toxins (e.g. a cytotoxic drug, or a radiotoxin).

In one aspect, the invention relates to drug conjugates (in particular antibody-drug conjugates ADCs) comprising the antigen binding proteins described herein conjugated to a toxin which further enhances efficient killing of specific cells, such as e.g., MAGE-A4 positive cells. The toxin moiety is typically a small molecular weight moiety, such as MMAE/MMAF, DM1, chaliceamicin, anthracycline toxins, taxol, gramicidin D and/or colchicine, which may be linked via a peptide linker to the antigen binding protein.

The toxin may be conjugated non-site-specifically or site-specifically to the antigen binding protein. Non-site-specific conjugation typically involves the use of chemical linkers, e.g., with maleimide functionality, that mediate conjugation to lysine or cysteine amino acid side chains of the antibody. Site-specific conjugation may be achieved using chemical, chemo-enzymatic, or enzymatic conjugations known in the art, e.g., employing bifunctional linkers, bacterial transglutaminase or sortase enzymes, linkers allowing Pictet-Spengler chemistry on formyl-glycine forming enzyme modified antigen binding proteins, or glycan-remodeled antigen binding proteins.

### Methods of Administering Antigen Binding Proteins

Methods of preparing and administering antigen binding proteins of the disclosure as well as the nucleic acids described herein, the vectors described herein, the host cell cells described herein or the compositions described herein to a subject are well known to or are readily determined by those skilled in the art. The route of administration of the antigen binding proteins of the current disclosure may e.g., be oral, parenteral, by inhalation, or topical. The term parenteral as used herein includes intravenous, intraarterial, intraperitoneal, intramuscular, subcutaneous, rectal or vaginal administration. The term intraocular as used herein includes, but is not limited to, subconjunctival, intravitreal, retrobulbar, or intracameral. The term topical as used herein includes, but is not limited to, administration with liquid or solution eye drops, emulsions (e.g., oil-in-water emulsions), suspensions, and ointments.

While all these forms of administration are clearly contemplated as being within the scope of the current disclosure, a form for administration would be a solution for injection. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Effective doses of the compositions of the present disclosure, for the treatment of the related conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins of the present disclosure, conjugates or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the antigen binding proteins shall be held to mean an amount sufficient to achieve effective binding to an antigen and to achieve a benefit, e.g., to ameliorate symptoms of a disease or disorder or to detect a substance or a cell. In the case of tumor cells, the antigen binding proteins will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antigen binding proteins of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antigen binding proteins described in the current disclosure may prove to be particularly effective. Similarly, the nucleic acids described herein, the vectors described herein, the host cell cells described herein (in particular the immune cells bearing a CAR) or the compositions described herein may be administered to a human or other animal in accordance with the methods of treatment described above in an amount sufficient to produce a therapeutic or prophylactic effect.

“Efficacy” or “in vivo efficacy” as used herein refers to the response to a therapy by the pharmaceutical composition of the disclosure, using e.g., standardized response criteria, such as standard ophthalmological response criteria. The success or in vivo efficacy of the therapy using a pharmaceutical composition of the disclosure refers to the effectiveness of the composition for its intended purpose, i.e., the ability of the composition to cause its desired effect. The in vivo efficacy may be monitored by established standard methods for the specific diseases. In addition, various disease specific clinical chemistry parameters and other established standard methods may be used.

In some embodiments, the compounds and cells described herein are administered in combination with one or more different pharmaceutical compounds. Generally, therapeutic use of the compounds and cells described herein may be in combination with one or more therapies selected from the group of antibody therapy, chemotherapy, cytokine therapy, dendritic cell therapy, gene therapy, hormone therapy, laser light therapy, radiation therapy or vaccine therapy.

### Methods of Treating Cancer or Viral Infections

Provided herein are methods of treating cancer or viral infections with the antigen binding proteins of the disclosure (e.g., an antigen binding protein comprising a Fab domain which binds a cell surface protein of an immune cell linked to a first and second pMHC binding domain). In certain embodiments, the cancer is caused by a viral infection.

In certain embodiments of the antigen binding protein of the disclosure, the target pMHC binding domain specifically targets an MHC restricted peptide derived of a tumor antigen or a viral antigen.

In one aspect, the disclosure provides a method for killing a target cell comprising a major histocompatibility complex (MHC) presenting a neoantigen, the method comprising: a) contacting a plurality of cells comprising immune cells and the target cell with the antigen binding protein described above, wherein said antigen binding protein specifically binds to the pMHC on the surface of the target cell and to CD3 on the surface of the immune cells; b) forming a specific binding complex through the antigen binding protein interactions with the target cells and the immune cells, thereby activating the immune cells; and c) killing the target cell with the activated immune cells.

In one aspect, the disclosure provides a method of treating cancer comprising the step of administering the antigen binding protein described above to a patient in need thereof.

### Kits

Also contemplated are kits comprising at least one nucleic acid library or antigen binding protein as described herein, typically together with a packaged combination of reagents with instructions. In one embodiment, the kit includes a composition containing an effective amount of said antigen binding protein in unit dosage form. Such kit may comprise a sterile container comprising the composition; non-limiting examples of such containers include, without being limited to, vials, ampoules, bottles, tubes, syringes, blister-packs. In some embodiments, the composition is a pharmaceutical composition and the containers is made of a material suitable for holding medicaments. In one embodiment, the kit may comprise in a first container the antigen binding protein in lyophilized form and a second container with a diluent (e.g., sterile water) for reconstitution or dilution of the antigen binding protein. In some embodiments, said diluent is a pharmaceutically acceptable diluent. In one embodiment, the kit is for diagnostic purposes and the antigen binding protein is formulated for diagnostic applications. In one embodiment, the kit is for therapeutic purposes and the antigen binding protein is formulated for therapeutic applications.

Typically, the kit will further comprise a separate sheet, pamphlet or card supplied in or with the container with instructions for use. If the kit is intended for pharmaceutical use, it may further comprise one or more of the following: information for administering the composition to a subject having a related disease or disorder and a dosage schedule, description of the therapeutic agent, precautions, warnings, indications, counter-indications, overdosage information and/or adverse reactions.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

TABLE 4
- Antigen binding protein amino acid
sequences. CDR sequences are highlighted
in bold underlined text.

| SEQ ID | Com- | |
|---|---|---|
| NO. | pound | Sequence |
| SEQ ID | CDR-1 | >CDR-1_HC |
| NO.: 40 | | EVQLVESGGGSVQPGGSLRLSCAASG |
| | | FTFSTYAMNWVRQAPGKGLEWVGRIRSKA |
| | | NNYATYYADSVKGRFTISRDDSKNTLYLQM |
| | | NSLRAEDTATYYCVRHGNFGDSYVSWFAY |
| | | WGQGTTVTVSSASTKGPSVFPLAPSSKSTSG |
| | | GTAALGCLVKDYFPEPVTVSWNSGALTSGV |
| | | HTFPAVLQSSGLYSLSSVVTVPSSSLGTQTYI |
| | | CNVNHKPSNTKVDKRVEPKSC |
| SEQ ID | | >CDR-1_LC |
| NO.: 41 | | EVQLVESGGGLAQAGGSLRVSCVASGRPFT |
| | | KYAWGWFRQAPGKAREFVATITWDGGKT |
| | | DYADSVKGRFTISKDSAENSIYLQMNSLKPE |
| | | DTAVYYCAADRNYCVGHRCYVRPDDYDY |
| | | WGQGTQVTVSSGGGGSAVVTQEPSLTVSPG |
| | | GTVTLTCGSSTGAVTTSNYANWVQQKPGK |
| | | SPRGLIGGTNKRAPGVPARFSGSLLGGKAA |
| | | LTISGAQPEDEADYYCALWYSNHWVFGGG |
| | | TKLTVLGTVAAPSVFIFPPSDEQLKSGTASV |
| | | VCLLNNFYPREAKVQWKVDNALQSGNSQE |
| | | SVTEQDSKDSTYSLSSTLTLSKADYEKHKVY |
| | | ACEVTHQGLSSPVTKSFNRGEC |
| SEQ ID | CDR-2 | >CDR-2_HC |
| NO.: 42 | | EVQLVESGGGSVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTATYYCVRHGNFGDSYVSWFAYWGQ |
| | | GTTVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSC |
| SEQ ID | | >CDR-2_LC |
| NO.: 43 | | AVVTQEPSLTVSPGGTVTLTCGSSTGAVTTS |
| | | NYANWVQQKPGKSPRGLIGGTNKRAPGVP |
| | | ARFSGSLLGGKAALTISGAQPEDEADYYCA |
| | | LWYSNHWVFGGGTKLTVLGTVAAPSVFIFP |
| | | PSDEQLKSGTASVVCLLNNFYPREAKVQWK |
| | | VDNALQSGNSQESVTEQDSKDSTYSLSSTLT |
| | | LSKADYEKHKVYACEVTHQGLSSPVTKSFN |
| | | RGECGGGGSEVQLVESGGGLAQAGGSLRVS |
| | | CVASGRPFTKYAWGWFRQAPGKAREFVAT |
| | | ITWDGGKTDYADSVKGRFTISKDSAENSIY |
| | | LQMNSLKPEDTAVYYCAADRNYCVGHRC |
| | | YVRPDDYDYWGQGTQVTVSS |
| SEQ ID | CDR-3 | >CDR-3_HC |
| NO.: 44 | | EVQLVESGGGSVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTATYYCVRHGNFGDSYVSWFAYWGQ |
| | | GTTVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLAQAGGSLRVSCVASGRPFTKYAWGW |
| | | FRQAPGKAREFVATITWDGGKTDYADSVK |
| | | GRFTISKDSAENSIYLQMNSLKPEDTAVYYC |
| | | AADRNYCVGHRCYVRPDDYDYWGQGTQ |
| | | VTVSS |
| SEQ ID | | >CDR-3_LC |
| NO.: 45 | | AVVTQEPSLTVSPGGTVTLTCGSSTGAVTTS |
| | | NYANWVQQKPGKSPRGLIGGTNKRAPGVP |
| | | ARFSGSLLGGKAALTISGAQPEDEADYYCA |
| | | LWYSNHWVFGGGTKLTVLGTVAAPSVFIFP |
| | | PSDEQLKSGTASVVCLLNNFYPREAKVQWK |
| | | VDNALQSGNSQESVTEQDSKDSTYSLSSTLT |
| | | LSKADYEKHKVYACEVTHQGLSSPVTKSFN |
| | | RGEC |
| SEQ ID | CDR-4 | >CDR-4_HC |
| NO.: 46 | | EVQLVESGGGLAQAGGSLRVSCVASGRPFT |
| | | KYAWGWFRQAPGKAREFVATITWDGGKT |
| | | DYADSVKGRFTISKDSAENSIYLQMNSLKPE |
| | | DTAVYYCAADRNYCVGHRCYVRPDDYDY |
| | | WGQGTQVTVSSGGGGSEVQLVESGGGSVQ |
| | | PGGSLRLSCAASGFTFSTYAMNWVRQAPGK |
| | | GLEWVGRIRSKANNYATYYADSVKGRFTIS |
| | | RDDSKNTLYLQMNSLRAEDTATYYCVRHG |
| | | NFGDSYVSWFAYWGQGTTVTVSSASTKGP |
| | | SVFPLAPSSKSTSGGTAALGCLVKDYFPEPV |
| | | TVSWNSGALTSGVHTFPAVLQSSGLYSLSSV |
| | | VTVPSSSLGTQTYICNVNHKPSNTKVDKRVE |
| | | PKSC |
| SEQ ID | | >CDR-4_LC |
| NO.: 47 | | AVVTQEPSLTVSPGGTVTLTCGSSTGAVTTS |
| | | NYANWVQQKPGKSPRGLIGGTNKRAPGVP |
| | | ARFSGSLLGGKAALTISGAQPEDEADYYCA |
| | | LWYSNHWVFGGGTKLTVLGTVAAPSVFIFP |
| | | PSDEQLKSGTASVVCLLNNFYPREAKVQWK |
| | | VDNALQSGNSQESVTEQDSKDSTYSLSSTLT |
| | | LSKADYEKHKVYACEVTHQGLSSPVTKSFN |
| | | RGEC |
| SEQ ID | CDR-5 | >CDR-5_HC |
| NO.: 48 | | EVQLVESGGGSVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTATYYCVRHGNFGDSYVSWFAYWGQ |
| | | GTTVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLAQAGGSLRVSCVASGRPFTKYAWGW |
| | | FRQAPGKAREFVATITWDGGKTDYADSVK |
| | | GRFTISKDSAENSIYLQMNSLKPEDTAVYYC |
| | | AADRNYCVGHRCYVRPDDYDYWGQGTQ |
| | | VTVSS> |
| SEQ ID | | >CDR-5_LC |
| NO.: 49 | | EVQLVESGGGLAQAGGSLRVSCVASGRPFT |
| | | KYAWGWFRQAPGKAREFVATITWDGGKT |
| | | DYADSVKGRFTISKDSAENSIYLQMNSLKPE |
| | | DTAVYYCAADRNYCVGHRCYVRPDDYDY |
| | | WGQGTQVTVSSGGGGSAVVTQEPSLTVSPG |
| | | GTVTLTCGSSTGAVTTSNYANWVQQKPGK |
| | | SPRGLIGGTNKRAPGVPARFSGSLLGGKAA |
| | | LTISGAQPEDEADYYCALWYSNHWVFGGG |
| | | TKLTVLGTVAAPSVFIFPPSDEQLKSGTASV |
| | | VCLLNNFYPREAKVQWKVDNALQSGNSQE |
| | | SVTEQDSKDSTYSLSSTLTLSKADYEKHKVY |
| | | ACEVTHQGLSSPVTKSFNRGEC |
| SEQ ID | CDR-6 | >CDR-6_HC |
| NO.: 50 | | EVQLVESGGGSVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTATYYCVRHGNFGDSYVSWFAYWGQ |
| | | GTTVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLAQAGGSLRVSCVASGRPFTKYAWGW |
| | | FRQAPGKAREFVATITWDGGKTDYADSVK |
| | | GRFTISKDSAENSIYLQMNSLKPEDTAVYYC |
| | | AADRNYCVGHRCYVRPDDYDYWGQGTQ |
| | | VTVSS> |
| SEQ ID | | >CDR-6_LC |
| NO.: 51 | | AVVTQEPSLTVSPGGTVTLTCGSSTGAVTTS |
| | | NYANWVQQKPGKSPRGLIGGTNKRAPGVP |
| | | ARFSGSLLGGKAALTISGAQPEDEADYYCA |
| | | LWYSNHWVFGGGTKLTVLGTVAAPSVFIFP |
| | | PSDEQLKSGTASVVCLLNNFYPREAKVQWK |
| | | VDNALQSGNSQESVTEQDSKDSTYSLSSTLT |
| | | LSKADYEKHKVYACEVTHQGLSSPVTKSFN |
| | | RGECGGGGSEVQLVESGGGLAQAGGSLRVS |
| | | CVASGRPFTKYAWGWFRQAPGKAREFVAT |
| | | ITWDGGKTDYADSVKGRFTISKDSAENSIY |
| | | LQMNSLKPEDTAVYYCAADRNYCVGHRC |
| | | YVRPDDYDYWGQGTQVTVSS |
| SEQ ID | CDR-7 | >CDR-7_HC |
| NO.: 52 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATRPSSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTYSAFNLWGQGTSV |
| | | TVSS |
| SEQ ID | | >CDR-7_LC |
| NO.: 53 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGQPKAAPSVT |
| | | LFPPSSEELQANKATLVCLISDFYPGAVTVA |
| | | WKADSSPVKAGVETTTPSKQSNNKYAASSY |
| | | LSLTPEQWKSHRSYSCQVTHEGSTVEKTVA |
| | | PTECS |
| SEQ ID | CDR-8 | >CDR-8_HC |
| NO.: 54 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATRPSSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTYSAFNLWGQGTSV |
| | | TVSS |
| SEQ ID | | >CDR-8_LC |
| NO.: 55 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGQPKAAPSVT |
| | | LFPPSSEELQANKATLVCLISDFYPGAVTVA |
| | | WKADSSPVKAGVETTTPSKQSNNKYAASSY |
| | | LSLTPEQWKSHRSYSCQVTHEGSTVEKTVA |
| | | PTECSGGGGSSYELTQPPSVSVSPGQTASITC |
| | | TADTLSRSYASWYQQKPGQSPVLVIYRDTS |
| | | RPSGIPERFSGSNSGNTATLTISGTQAMDEA |
| | | DYYCATRPSSGSNFQLFGGGTKLTVLGGGG |
| | | GGSGGGGSGGGGSGGGGSESQVLESGGGSV |
| | | QPGGSLRLSCTVSGFSLSNYAMSWVRQAPG |
| | | KGLEYIGIVSSGGTTYYASWAKGRFTISKD |
| | | TSKNTVYLQMNSLRAEDTASYYCAKDLYY |
| | | GPTTYSAFNLWGQGTSVTVSS |
| SEQ ID | CDR-9 | >CDR-9_HC |
| NO.: 56 | | EVQLVESGGGLVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLVQPGGSLRLSCVASGRPFTKYAWGW |
| | | FRQAPGKAREFVATITWDGGKTDYADSVK |
| | | GRFTISKDSAKNSIYLQMNSLRAEDTAVYYC |
| | | AADRNYCVGHRCYVRPDDYDYWGQGTLV |
| | | TVSS |
| SEQ ID | | >CDR-9_LC |
| NO.: 57 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSEVQLVESGGGLVQPGGSLRL |
| | | SCVASGRPFTKYAWGWFRQAPGKAREFVA |
| | | TITWDGGKTDYADSVKGRFTISKDSAKNSI |
| | | YLQMNSLRAEDTAVYYCAADRNYCVGHR |
| | | CYVRPDDYDYWGQGTLVTVS |
| SEQ ID | CDR-10 | >CDR-10_HC |
| NO.: 58 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLVQPGGSLRLSCVASGRPFTKYAWGW |
| | | FRQAPGKAREFVATITWDGGKTDYADSVK |
| | | GRFTISKDSAKNSIYLQMNSLRAEDTAVYYC |
| | | AADRNYCVGHRCYVRPDDYDYWGQGTLV |
| | | TVSS |
| SEQ ID | | >CDR-10_LC |
| NO.: 59 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSEVQLVESGGGLVQPGGSLRL |
| | | SCVASGRPFTKYAWGWFRQAPGKAREFVA |
| | | TITWDGGKTDYADSVKGRFTISKDSAKNSI |
| | | YLQMNSLRAEDTAVYYCAADRNYCVGHR |
| | | CYVRPDDYDYWGQGTLVTVSS |
| SEQ ID | CDR-11 | >CDR-11_HC |
| NO.: 60 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGNSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSEVQLVES |
| | | GGGLVQPGGSLRLSCVASGRPFTKYAWGW |
| | | FRQAPGKAREFVATITWDGGKTDYADSVK |
| | | GRFTISKDSAKNSIYLQMNSLRAEDTAVYYC |
| | | AADRNYCVGHRCYVRPDDYDYWGQGTLV |
| | | TVSS |
| SEQ ID | | >CDR-11_LC |
| NO.: 61 | | QAVVTQEPSLTVSPGGTVTLTCRSSTGAVT |
| | | TSNYANWVQQKPGQAPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSEVQLVESGGGLVQPGGSLRL |
| | | SCVASGRPFTKYAWGWFRQAPGKAREFVA |
| | | TITWDGGKTDYADSVKGRFTISKDSAKNSI |
| | | YLQMNSLRAEDTAVYYCAADRNYCVGHR |
| | | CYVRPDDYDYWGQGTLVTVSS |
| SEQ ID | CDR-12 | >CDR-12_HC |
| NO.: 62 | | EVQLVESGGGLVQPGGSLRLSCAASGFTFST |
| | | YAMNWVRQAPGKGLEWVGRIRSKANNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATSDGSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTYSAFNLWGQGTSV |
| | | TVSS |
| SEQ ID | | >CDR-12_LC |
| NO.: 63 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSSYELTQPPSVSVSPGQTASIT |
| | | CTADTLSRSYASWYQQKPGQSPVLVIYRDT |
| | | SRPSGIPERFSGSNSGNTATLTISGTQAMDEA |
| | | DYYCATSDGSGSNFQLFGGGTKLTVLGGG |
| | | GGGSGGGGSGGGGSGGGGSESQVLESGGGS |
| | | VQPGGSLRLSCTVSGFSLSNYAMSWVRQAP |
| | | GKGLEYIGIVSSGGTTYYASWAKGRFTISK |
| | | DTSKNTVYLQMNSLRAEDTASYYCAKDLY |
| | | YGPTTYSAFNLWGQGTSVTVSS |
| SEQ ID | CDR-13 | >CDR-13_HC |
| NO.: 64 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATSDGSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTYSAFNLWGQGTSV |
| | | TVSS |
| SEQ ID | | >CDR-13_LC |
| NO.: 65 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSSYELTQPPSVSVSPGQTASIT |
| | | CTADTLSRSYASWYQQKPGQSPVLVIYRDT |
| | | SRPSGIPERFSGSNSGNTATLTISGTQAMDEA |
| | | DYYCATSDGSGSNFQLFGGGTKLTVLGGG |
| | | GGGSGGGGSGGGGSGGGGSESQVLESGGGS |
| | | VQPGGSLRLSCTVSGFSLSNYAMSWVRQAP |
| | | GKGLEYIGIVSSGGTTYYASWAKGRFTISK |
| | | DTSKNTVYLQMNSLRAEDTASYYCAKDLY |
| | | YGPTTYSAFNLWGQGTSVTVSS |
| SEQ ID | CDR-14 | >CDR-14_HC |
| NO.: 66 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGNSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATSDGSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTYSAFNLWGQGTSV |
| | | TVSS |
| SEQ ID | | >CDR-14_LC |
| NO.: 67 | | QAVVTQEPSLTVSPGGTVTLTCRSSTGAVT |
| | | TSNYANWVQQKPGQAPRGLIGGTNKRAPG |
| | | VPARFSGSLLGGKAALTISGAQPEDEADYYC |
| | | ALWYSNHWVFGGGTKLTVLGTVAAPSVFIF |
| | | PPSDEQLKSGTASVVCLLNNFYPREAKVQW |
| | | KVDNALQSGNSQESVTEQDSKDSTYSLSSTL |
| | | TLSKADYEKHKVYACEVTHQGLSSPVTKSF |
| | | NRGECGGGGSSYELTQPPSVSVSPGQTASIT |
| | | CTADTLSRSYASWYQQKPGQSPVLVIYRDT |
| | | SRPSGIPERFSGSNSGNTATLTISGTQAMDEA |
| | | DYYCATSDGSGSNFQLFGGGTKLTVLGGG |
| | | GGGSGGGGSGGGGSGGGGSESQVLESGGGS |
| | | VQPGGSLRLSCTVSGFSLSNYAMSWVRQAP |
| | | GKGLEYIGIVSSGGTTYYASWAKGRFTISK |
| | | DTSKNTVYLQMNSLRAEDTASYYCAKDLY |
| | | YGPTTYSAFNLWGQGTSVTVSS |
| SEQ ID | CDR-15 | >CDR-15_HC |
| NO.: 68 | | EVQLVESGGGLVQPGGSLRLSCAASGFTFST |
| | | YAMNWVRQAPGKGLEWVGRIRSKYNNYA |
| | | TYYADSVKGRFTISRDDSKNTLYLQMNSLR |
| | | AEDTAVYYCVRHGNFGDSYVSWFAYWGQ |
| | | GTLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICNV |
| | | NHKPSNTKVDKRVEPKSCGGGGSSYELTQP |
| | | PSVSVSPGQTASITCTADTLSRSYASWYQQK |
| | | PGQSPVLVIYRDTSRPSGIPERFSGSNSGNTA |
| | | TLTISGTQAMDEADYYCATSDGSGSNFQLF |
| | | GGGTKLTVLGGGGGGSGGGGSGGGGSGGG |
| | | GSESQVLESGGGSVQPGGSLRLSCTVSGFSL |
| | | SNYAMSWVRQAPGKGLEYIGIVSSGGTTYY |
| | | ASWAKGRFTISKDTSKNTVYLQMNSLRAED |
| | | TASYYCAKDLYYGPTTQSAFNLWGQGTSV |
| | | TVSS |
| SEQ ID | | >CDR-15_LC |
| NO.: 69 | | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYYC |
| | | ALWYSNHWVFGGGTKLTVLGQPKAAPSVT |
| | | LFPPSSEELQANKATLVCLISDFYPGAVTVA |
| | | WKADSSPVKAGVETTTPSKQSNNKYAASSY |
| | | LSLTPEQWKSHRSYSCQVTHEGSTVEKTVA |
| | | PTECSGGGGSSYELTQPPSVSVSPGQTASITC |
| | | TADTLSRSYASWYQQKPGQSPVLVIYRDTS |
| | | RPSGIPERFSGSNSGNTATLTISGTQAMDEA |
| | | DYYCATSDGSGSNFQLFGGGTKLTVLGGG |
| | | GGGSGGGGSGGGGSGGGGSESQVLESGGGS |
| | | VQPGGSLRLSCTVSGFSLSNYAMSWVRQAP |
| | | GKGLEYIGIVSSGGTTYYASWAKGRFTISK |
| | | DTSKNTVYLQMNSLRAEDTASYYCAKDLY |
| | | YGPTTQSAFNLWGQGTSVTVSS |
| SEQ ID | sTCR | >alpha_chain_sTCR_comparator |
| NO.: 70 | compar- | MANQVEQSPQSLIILEGKNVTLQCNYTVSPF |
| | ator | SNLRWYKQDTGRGPVSLTILDYAINTKSNG |
| | | RYTATLDADTKQSSLHITASQLSDSASYICV |
| | | VNRADGLYIPTFGRGTSLIVHPYIQKPDPAV |
| | | YQLRDSKSSDKSVCLFTDFDSQTNVSQSKDS |
| | | DVYITDKCVLDMRSMDFKSNSAVAWSNKS |
| | | DFACANAFNNSIIPEDT |
| SEQ ID | | >beta_chain_sTCR_comparator |
| NO.: 71 | | MAIQMTQSPSSLSASVGDRVTITCRASQDIR |
| | | NYLNWYQQKPGKAPKLLIYYTSRLESGVPS |
| | | RFSGSGSGTDYTLTISSLQPEDFATYYCQQG |
| | | NTLPWTFGQGTKVEIKGGGGSGGGGSGGGG |
| | | SGGGGSGGGGSEVQLVESGGGLVQPGGSLR |
| | | LSCAASGYSFTGYTMNWVRQAPGKGLEWV |
| | | ALINPYKGVSTYNQKFKDRFTISVDKSKNTA |
| | | YLQMNSLRAEDTAVYYCARSGYYGDSDWY |
| | | FDVWGQGTLVTVSSGGGGSDVKVTQSSRYL |
| | | VKRTGEKVFLECVQDAPLSKMFWYRQDPG |
| | | LGLRLIYFSYDVKLKEKGDIPEGYSVSREKK |
| | | ERFSLILESASTNQTSMYLCASSSDQNSGDP |
| | | YEQYFGPGTRLTVTEDLKNVFPPEVAVFEPS |
| | | EAEISHTQKATLVCLATGFYPDHVELSWWV |
| | | NGKEVHSGVCTDPQPLKEQPALNDSRYALS |
| | | SRLRVSATFWQDPRNHFRCQVQFYGLSEND |
| | | EWTQDRAKPVTQIVSAEAWGRAD |
| SEQ ID | CDR-16 | >CDR-16 |
| NO.: 72 | | EVQLVESGGGSAQAGGSLRVSCVASGRPFT |
| | | KYAWGWFRQAPGKAREFVATITWDGGKTD |
| | | YADSVKGRFTISKDSAENSIYLQMNSLKPED |
| | | TASYYCAADRNYCVGHRCYVRPDDYDYW |
| | | GQGTSVTVSSA |
| SEQ ID | CDR-17 | >CDR-17 |
| NO.: 73 | | EVQLVESGGGLVQPGGSLRLSCVASGRPFTK |
| | | YAWGWFRQAPGKAREFVATITWDGGKTDY |
| | | ADSVKGRFTISKDSAKNSIYLQMNSLRAEDT |
| | | AVYYCAADRNYCVGHRCYVRPDDYDYWG |
| | | QGTLVTVSS |
| SEQ ID | CDR-18 | >CDR-18_HC |
| NO.: 74 | | EVQLVESGGGLVQPGGSLRLSCAASGFNIKD |
| | | TYIHWVRQAPGKGLEWVARIYPTNGYTRYA |
| | | DSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNT |
| | | KVDKRVEPKSC |
| SEQ ID | | >CDR-18_LC |
| NO.: 75 | | DIQMTQSPSSLSASVGDRVTITCRASQDVNT |
| | | AVAWYQQKPGKAPKLLIYSASFLYSGVPSR |
| | | FSGSRSGTDFTLTISSLQPEDFATYYCQQHYT |
| | | TPPTFGQGTKVEIKRTVAAPSVFIFPPSDEQL |
| | | KSGTASVVCLLNNFYPREAKVQWKVDNAL |
| | | QSGNSQESVTEQDSKDSTYSLSSTLTLSKAD |
| | | YEKHKVYACEVTHQGLSSPVTKSFNRGECG |
| | | GGGSQAVVTQEPSLTVSPGGTVTLTCRSSTG |
| | | AVTTSNYANWVQQKPGQAPRGLIGGTNKR |
| | | APGVPARFSGSLLGGKAALTISGAQPEDEAD |
| | | YYCALWYSNHWVFGGGTKLTVLGGGGGSG |
| | | GGGSGGGGSGGGGSEVQLVESGGGSVQPG |
| | | GSLRLSCAASGFTFSTYAMNWVRQAPGKGL |
| | | EWVGRIRSKYNNYATYYADSVKGRFTISRD |
| | | DSKNTLYLQMNSLRAEDTAMYYCVRHGNF |
| | | GNSYVSWFAYWGQGTTVTVSS |
| SEQ ID | CD3 | STYAMN |
| NO.: 76 | CDRH1 | |
| SEQ ID | CD3 | RIRSKANNYATYYADSVKG |
| NO.: 77 | CDRH2 | |
| SEQ ID | CD3 | HGNFGDSYVSWFAY |
| NO.: 78 | CDRH3 | |
| SEQ ID | CD3 | GSSTGAVTTSNYAN |
| NO.: 79 | CDRL1_ | |
| | a | |
| SEQ ID | CD3 | RSSTGAVTTSNYAN |
| NO.: 80 | CDRL1_ | |
| | b | |
| SEQ ID | CD3 | GTNKRAP |
| NO.: 81 | CDRL2 | |
| SEQ ID | CD3 | ALWYSNHWV |
| NO.: 82 | CDRL3 | |
| SEQ ID | VL | AVVTQEPSLTVSPGGTVTLTCRSSTGAVTTS |
| NO.: 83 | CD3_a | NYANWVQQKPGKSPRGLIGGTNKRAPGVP |
| | | ARFSGSLLGGKAALTISGAQPEDEADYYCAL |
| | | WYSNHWVFGGGTKLTVLG |
| SEQ ID | VH | EVQLVESGGGSVQPGGSLRLSCAASGFTFST |
| NO.: 84 | CD3_a | YAMNWVRQAPGKGLEWVGRIRSKFNNYAT |
| | | YYADSVKGRFTISRDDSKNTLYLQMNSLRA |
| | | EDTATYYCVRHGNFGDSYVSWFAYWGQGT |
| | | TVTVSS |
| SEQ ID | VL | AVVTQEPSLTVSPGGTVTLTCGSSTGAVTTS |
| NO.: 85 | CD3_b | NYANWVQQKPGKSPRGLIGGTNKRAPGVP |
| | | ARFSGSLLGGKAALTISGAQPEDEADYYCAL |
| | | WYSNHWVFGGGTKLTVL |
| SEQ ID | VH | EVQLVESGGGSVQPGGSLRLSCAASGFTFST |
| NO.: 86 | CD3_b | YAMNWVRQAPGKGLEWVGRIRSKANNYA |
| | | TYYADSVKGRFTISRDDSKNTLYLQMNSLR |
| | | AEDTATYYCVRHGNFGDSYVSWFAYWGQG |
| | | TTVTVSS |

### EXAMPLES

Intracellular tumor antigens presented as peptides on MHC (pMHC) class I molecules are attractive targets for more tumor-selective immunotherapeutic approaches with promising data already emerging from clinical trials. pMHCs have been targeted by TCR-engineered T cells or soluble recombinant T-cell receptors (TCRs) fused to an anti-CD3 fragment. Naturally occurring cancer reactive TCRs have weak affinity and require substantial affinity enhancements for their cognate pMHC. However, the outcome of this process is difficult to predict and bears the risk for off-target cross reactivities in normal tissues, which may lead to severe adverse events in the clinic.

Here, we describe highly potent antigen binding proteins having a dual pMHC T-cell engager (“TCE”) format with high specificity towards tumor-specific pMHCs utilizing the HLA-A*02:01 restricted MAGE-A4 epitope GVYDGREHTV (SEQ ID NO.: 1). A series of monovalent and bivalent antibody constructs composed of anti-MAGE-A4 binding arms, ranging in affinities from 30 nM to 100 pM, were fused to an anti-CD3 Fab fragment with lower affinity compared to that commonly used for TCR-fusions. The different antibody constructs were evaluated for selective killing of MAGE-A4/HLA-A*02 positive human U2OS osteosarcoma and A375 melanoma cancer cells versus a panel of different MAGE-A4-negative/HLA-A*02-positive human cell lines. Bivalent bispecific antibody variants mediated at least a 7-fold greater degree of cancer cell killing and similarly increased T cell activation compared to their monovalent bispecific counterparts. IC50 values ranged as low as single digit picomolar, while the overall cross reactivity against MAGE-A4-negative/HLA-A*02-positive cells was not substantially affected. These results prove that dual targeting of pMHCs on cancer cells provides selective and efficient T cell-mediated target cell killing and T cell activation, even at very low levels of pMHC on the cell surface, highlighting the pivotal roles played by the affinity of the individual arms, valency, and epitope densities. The benefit of dual pMHC targeting was also tested for other than MAGE-A4/HLA-A*02 pMHCs. T cell engagers specific for two distinct cancer-derived pMHCs unrelated to MAGE-A4 were tested in cytotoxicity assays in mono- and bivalent formats. Alike MAGE-A4 targeting TCEs, dual engagers showed improved cancer cell killing, compared to their monovalent counterparts. The MAGE-A4/HLA-A*02:01-targeting dual pMHC TCE was optimized for CD3 affinity and MAGE-A4/HLA-A*02:01 target affinity to achieve high potency while maintaining specificity by minimizing binding to similar and physiologically relevant non-MAGE-A4 peptides (S1, S16). We analyzed the optimized dual pMHC TCE for potential off-target effects by recognition of similar and physiologically relevant non-MAGE-A4 peptides. T2 cells pulsed with similar peptides and co-cultured with PBMC effector cells showed no significant T cell activation or IFNg release in the presence of the dual pMHC TCE in comparison to MAGE-A4 peptide-pulsed T2 cells. Finally, we compared the potency, cytokine release, and specificity of the dual pMHC TCE against a recombinant TCR fused to an anti-CD3 scFv, a construct that is currently in clinical development. Interestingly, the dual pMHC TCE resulted in a 3-fold more potent cancer cell killing while having significantly lower effect on cytokine production. In conclusion, pMHC targeting with the dual pMHC TCEs described herein is an attractive alternative to soluble affinity-enhanced TCR-based cancer immunotherapies as they facilitate potent tumor targeting without the need for extensive affinity enhancements. The dual pMHC TCEs provided herein show (i) selective and efficient T cell-mediated target cell killing, (ii) effective activation of T-cells and (iii) lower cytokine release than comparator molecule. Dual pMHC targeting with the antigen binding proteins provided herein is highly potent while lower cytokine release may avoid T cell exhaustion, thus providing the promise of more effective and durable anticancer responses.

### Example 1—General Method for Production of Monovalent and Bivalent pMHC-Targeting T Cell Engagers

Bispecific antigen binding proteins as described in the examples below were expressed by transient co-transfection in HEK293-6E cells. Cells were cultured in suspension using polyethylenimine (PEI 40kD linear). HEK293-6E cells were seeded at 1.7×10⁶ cells/mL in Freestyle F17 medium supplemented with 2 mM L-Glutamine. DNA and PEI were added separately to 50 μL medium without supplement. Both fractions were mixed at 1:2.5 DNA:PEI ratio, vortexed and rested for 15 minutes. Cells and DNA/PEI mixture were combined (1 μg DNA/mL cells) and incubated at 37° C., 5% CO₂, 80% RH. After 24 hours, cells were supplemented with Tryptone N1 at 25 μL per mL production volume. After 7 days, cells were harvested by centrifugation and the supernatant was sterile filtered. The antigen binding proteins were purified by an affinity chromatography from the supernatant. Supernatant was loaded on a protein CH column (Thermo Fisher Scientific, #494320005) equilibrated with 6 CV PBS (pH 7.4). After a washing step with the same buffer, protein was eluted from the column by step elution with 100 mM Citric acid (pH 3.0). Fractions with the desired antigen binding protein were immediately neutralized by 1 M Tris Buffer (pH 9.0) at 1:10 ratio. Size exclusion chromatography was performed as an additional purification step. Samples were run on the Superdex 200 10/300 GL column with PBS (pH7.4) as a running buffer. Collected fractions were analyzed by SE-HPLC for monomer content and pooled accordingly. Final protein purity was assessed by SDS-PAGE and SE-HPLC.

### Example 2—General Methods for In Vitro Characterization of the Bispecific pMHC Targeting T Cell Engagers

Affinity characterization of HLA-A2/MAGE-A4×CD3 bispecific antibodies was performed by surface plasmon resonance (SPR). All experiments were conducted using a Biacore™ T200 Device (Cytiva). To determine the kinetic parameters of the binding of the bispecific antibodies to the HLA-A2/MAGE-A4 complex, a streptavidin chip (SAHC30M, XanTec) was coated according to the manufacturer's instructions with 500 RU HLA-A*02:01 in complex with the MAGE-A4 peptide. The resulting affinities presented herein correspond to the measurements performed with the respective monovalent antigen binding proteins. To determine the kinetic parameters of the bispecific antibodies to CD3, a HC30M chip (XanTec) was coated according to the manufacturer's instructions with 400 RU of CD3 heterodimer (Acro Biosystems). Uncoated channels were used for referencing. Data fitting was performed using a 1:1 Langmuir model.

To determine the in vitro cytotoxicity of the bispecific antibodies the Lactate Dehydrogenase (LDH) release assay was performed. Briefly, target cells were co-cultured with effector cells (i.e., PBMCs) at an E:T ratio of 10:1. Solutions of the bispecific antibodies covering a concentration range from 0.001 nM to 15 nM were added to the relevant wells. Cytotoxicity was quantified by colorimetric absorbance measurements of the amount of LDH released from damaged cells into the medium after 48 h. Cytokine release was determined after 24 h. Quantification of IL-2 and IFNg was performed using a respective cytokine ELISA kit (Invitrogen).

Thermal stability of the bispecific antibodies was measured using a differential scanning fluorimentry (DSF), as described in the Protein Thermal Shift manual MAN4461806B from Applied Biosystems (Thermo Fisher).

### Example 3—Generation and Characterization of Various Bispecific Antibody Formats

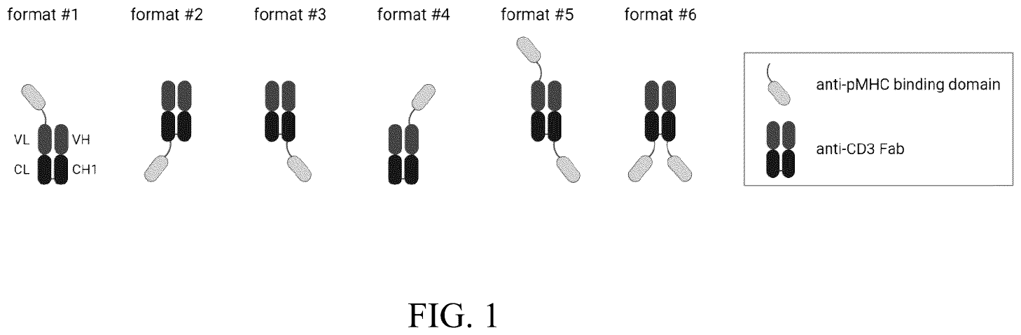

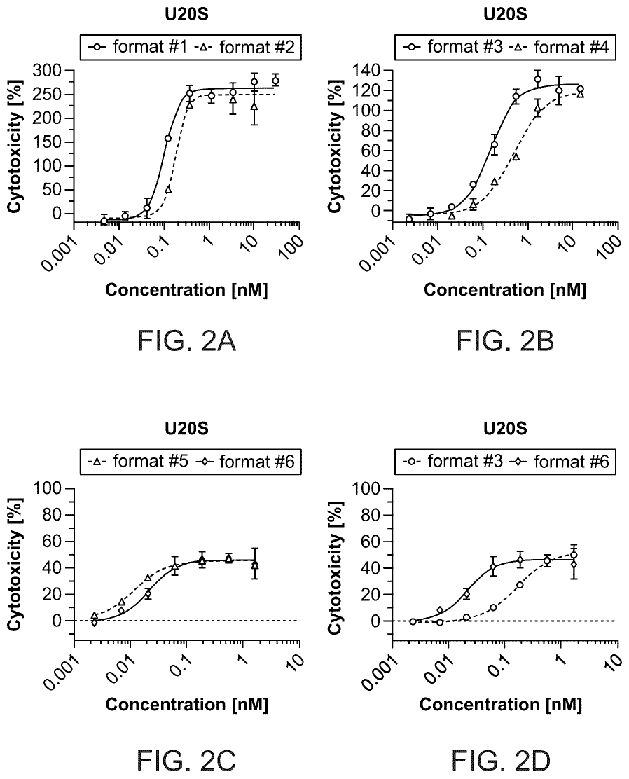

To determine the most optimal format of the bispecific molecule, rotation of the VHH MAGE-A4 binding moiety was performed on the N- and C-terminus of the light chain, and the C- and N-terminus of the heavy chain (formats 1-4 and respectively compounds CDR1, CDR-2, CDR-3 and CDR-4, FIG. 1) of a CD3 binding Fab. Monovalent bispecific T-cell engagers were tested for their affinity to MAGE-A4, as determined by SPR, in vitro potency, as determined by the LDH assay, and thermal stability, as determined by DSF. Results are summarized in Table 5. The respective T cell-mediated cytotoxicity results are shown in FIGS. 2A-B.

TABLE 5
Comparison of affinity, cytotoxicity, thermal stability,
and expression yield of HLA-A2/MAGE-A4-specific monovalent
bispecific antibodies in various Fab-VHH formats.

| | | α HLA- | U20S cell | Melting | Expres- |
|---|---|---|---|---|---|
| | | A2/MAGE- | killing, | temper- | sion |
| | | A4 K_{D} | IC50 | ature | yield |
| Compound | Format | (nM) | (nM) | (° C.) | (mg/L) |
| CDR-1 | Format #1 | 1.5 | 0.10 | 70.9 | 8.2 |
| CDR-2 | Format #2 | 2.6 | 0.18 | 71.3 | 8.2 |
| CDR-3 | Format #3 | 1.9 | 0.24 | 71.3 | 49 |
| CDR-4 | Format #4 | 1.3 | 0.50 | 70.7 | 10.6 |

Comparative analysis of the various monovalent bispecific antibody formats revealed that positioning of the MAGE-A4 binding moiety on the CD3 binding Fab impacted neither the affinity to the MAGE-A4 antigen nor the thermal stability of the construct. However, N-terminal heavy chain fusion of the MAGE-A4 binding arm (i.e., format #4) resulted in reduction of the in vitro efficacy, likely due to steric hindrance of the CD3 binding arm. In addition, a C-terminal fusion of the MAGE-A4 binding arm on the heavy chain of the CD3 binding arm resulted in over a 4-fold increase in the expression yield.

It was hypothesized that a bivalent pMHC-targeting T cell engager could mimic the natural avidity of T cells through the binding of two pMHC molecules on the surface of a single tumor cell (without being bound to theory). Thus, a dual (i.e., bivalent) pMHC-MAGE-A4-targeting T cell engager was compared against a monovalent pMHC-MAGE-A4-targeting T cell engager. For the bivalent bispecific constructs, a C-terminal fusion of the MAGE-A4 targeting VHH on the heavy chain in combination with N- or C-terminal fusion on the light chain were investigated (formats 5 and 6, compounds CDR-5 and CDR-6, respectively, FIG. 1). Comparison of the two bivalent bispecific formats in cytotoxicity assays, thermal stability and expression yield was performed. Results are summarized in Table 6. The respective T cell-mediated cytotoxicity results are shown in FIG. 2C. Formats #5 and #6 showed excellent in vitro efficacy, with IC50 values approximately 10-fold lower compared to the monovalent variants, confirming the superiority of the bivalent MAGE-A4 binding modus over the monovalent. Format #6 showed slightly higher thermal stability and better expression yield compared to format #5.

TABLE 6
Comparison of cytotoxicity, thermal stability and
expression yield of HLA-A2/MAGE-A4-specific bivalent
bispecific antibodies in various formats.

| | | U20S cell | Melting | Expression |
|---|---|---|---|---|
| | | killing, IC50 | temperature | yield |
| Compound | Format | (nM) | (° C.) | (mg/L) |
| CDR-5 | Format | 0.01 | 68.8 | 32.8 |
| | #5 | | | |
| CDR-6 | Format | 0.02 | 71.5 | 35.4 |
| | #6 | | | |

A direct comparison of the monovalent and the bivalent pMHC T cell engager (formats #3 and #6) was performed (FIG. 2D). The bivalent pMHC T cell engager confirmed superior cancer cell killing over its monovalent counterpart.

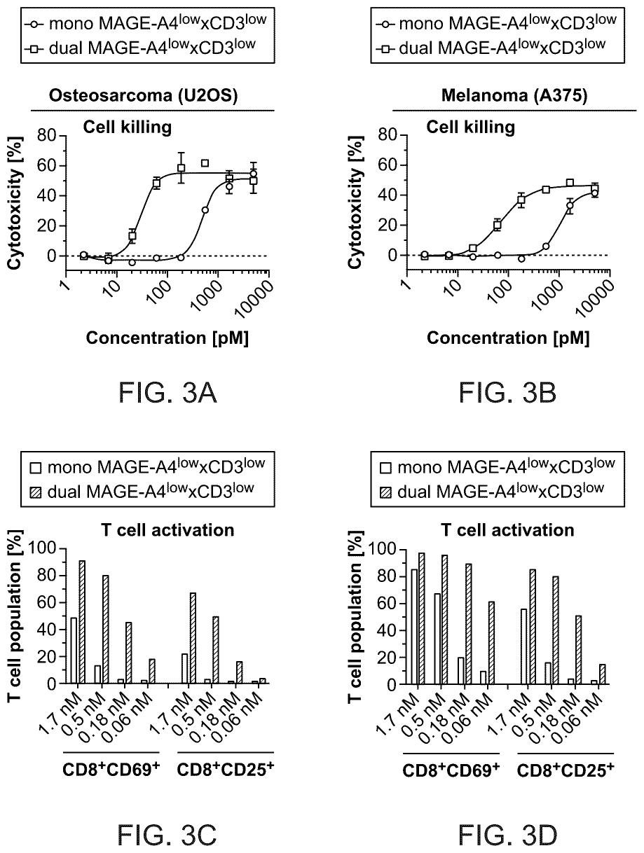

Finally, cytotoxicity in other cancer cell lines and the associated cytokine release profile were compared for the monovalent (compound CDR-3, format #3) and bivalent (compound CDR-6, format #6) pMHC T cell engagers. As shown in FIGS. 3A-3D, percent cancer cell killing was measured in osteosarcoma (U2OS) and melanoma (A375) cells incubated with a dual pMHC-targeting T cell engager or a single pMHC-targeting T cell engager comprising the same MAGE-A4 and CD3-binding antibody fragments. The MAGE-A4 and HLA-A*02 positive cell line U2OS (osteosarcoma) was incubated with human PBMCs at an E:T ratio of 10:1 (FIG. 3A). Similarly, MAGE-A4 and HLA-A*02 positive cell line A375 (melanoma) was incubated with human PBMCs at an E:T ratio of 10:1 (FIG. 3B). Cancer cell killing was measured at various concentrations of the two antigen binding proteins with an LDH release assay after 48 hours. The data shows a 10-fold increase in cancer cell killing potency with a dual pMHC-targeting T cell engager compared to a single pMHC-targeting T cell engager. T cell activation was determined by quantification of CD69 and CD25 markers on the CD8 T cell population after 24 h using flow cytometry (FIGS. 3 C-D), showing T cell activation on the U2OS (FIG. 3C) and the A375 (FIG. 3D) cell line, respectively. The bivalent Fab-(VHH)2 format of the MAGE-A4 targeting TCE shows superior cancer cell killing and T cell activation compared to its monovalent counterpart. Thus, bivalent targeting of antigen positive cancer cells greatly potentiates activity of the pMHC-targeting T-cell engagers. In this example, each antigen binding protein utilized a low affinity anti-CD3 Fab (see Example 4).

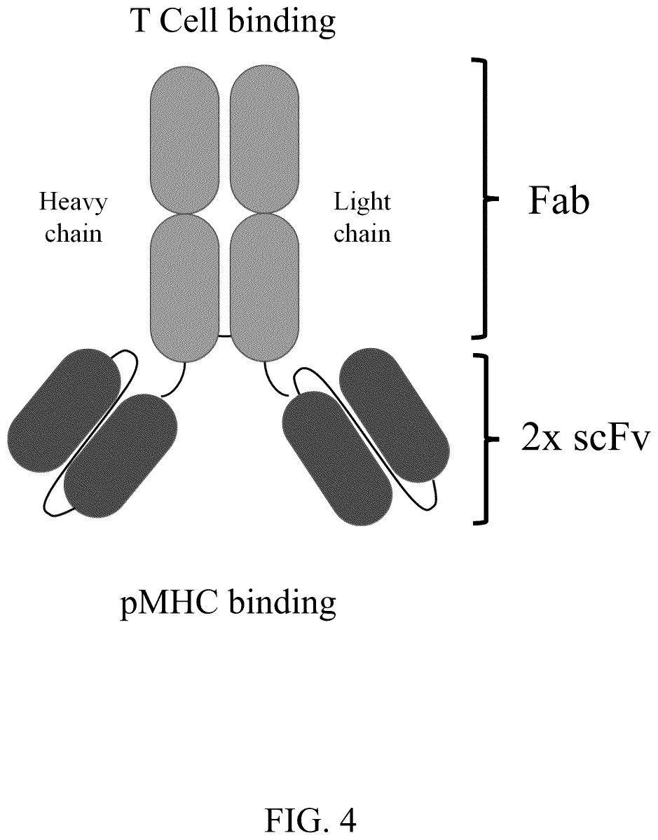

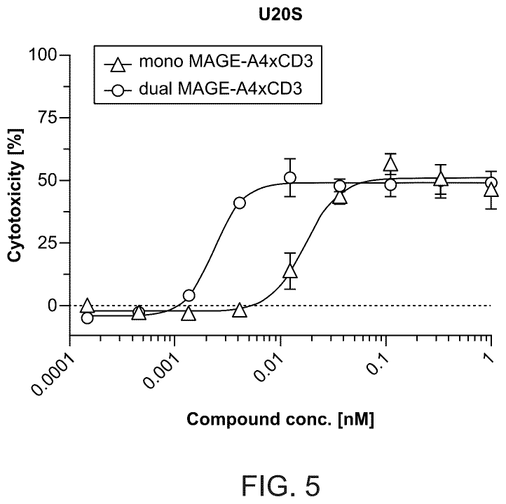

Further investigation of mono- and bivalent pMHC targeting TCEs in formats #3 and #6, where the pMHC binding moieties comprised scFvs was performed. Compounds tested were CDR-7 and CDR-8, respectively. Schematic representation of compound CDR-8, a dual engager with two pMHC-specific binding domains in a scFv format and a Fab domain targeting CD3 as a T cell recruiting domain is depicted in FIG. 4. Dual engager CDR-8 and its monovalent counterpart CDR-7 were tested for efficacy in LDH assay. MAGE-A4-positive HLA-A*02:01-positive osteosarcoma cell line U2OS was incubated with human PBMCs at an E:T ratio of 10:1. Cancer cell killing was measured at various concentrations of the compounds (FIG. 5). Again, data showed superiority of the dual T cell engager over its monovalent counterpart with about a 10-fold increase in cancer cell killing potency.

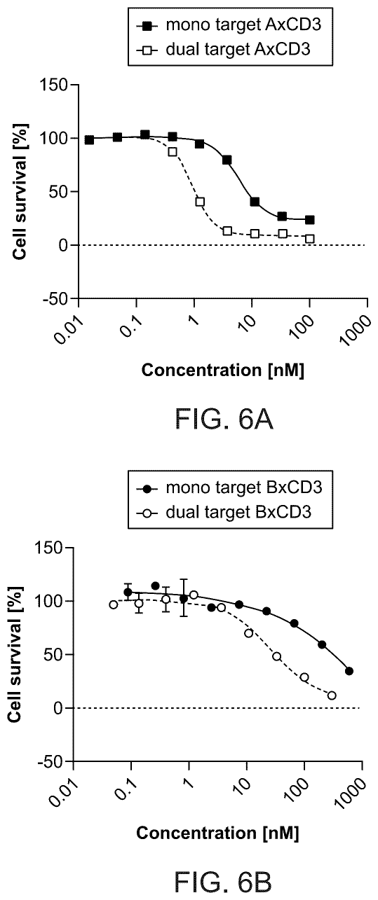

As further proof of concept, the benefit of bivalent targeting of pMHCs was also tested in other cancer cell lines and for other target pMHCs. Two different pMHC targeting T cell engagers targeting two distinct—pMHC antigens, i.e., target A and B, were generated in mono (i.e., Fab-scFv) and bivalent (i.e., Fab-(scFv)2) formats, and tested in cytotoxicity assays. Briefly, lung squamous cell carcinoma (expressing target A) and colorectal adenocarcinoma (expressing target B) cells were incubated with human PBMCs at an E:T ratio of 10:1 and varying concentrations of mono- and bivalent pMHC targeting TCEs. Cytotoxicity was measured after 48 h incubation using the CellTiter-Glo® Luminescent Cell Viability Assay (Promega), according to the manufacturer's instructions. Results are shown in FIGS. 6A-6B. Alike MAGE-A4 targeting TCEs, bivalent pMHC TCEs specific for other unrelated targets showed improved cancer cell killing, compared to their monovalent counterparts.

### Example 4—CD3 Affinity of the Bivalent pMHC-Targeting T Cell Engager Influences the T Cell-Mediated Cytotoxicity and the Corresponding Cytokine Release

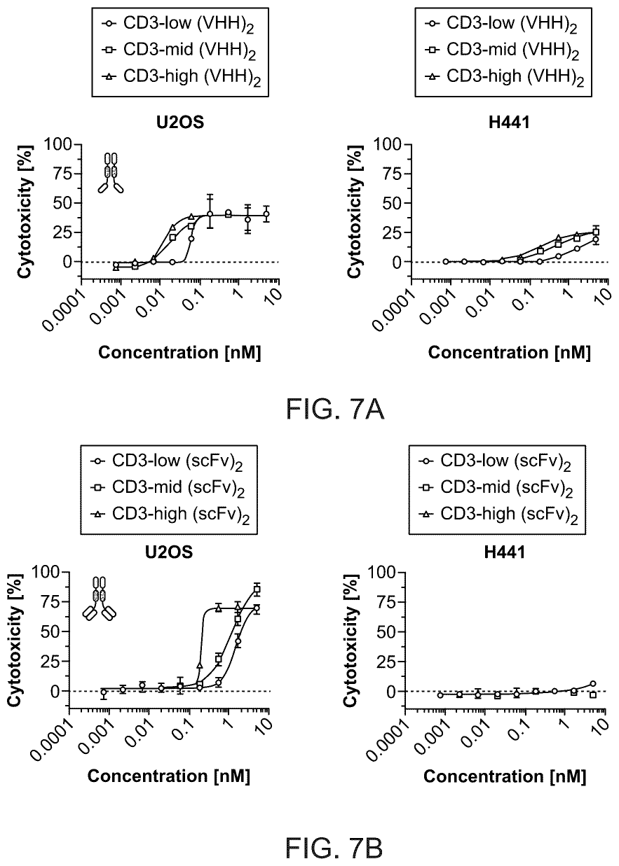

Dual pMHC T cell engagers with MAGE-A4 arms comprising two identical VHHs (FIG. 7A) or scFvs (FIG. 7B) with low (54 nM), mid (11 nM) and high (1.2 nM) CD3 affinity Fabs were tested in the LDH assay on MAGE-A4-positive U2OS cells and MAGE-A4-negative H441 cells. CDR-9, CDR-10 and CDR-11 comprised Fab-(VHH)2 compounds with low, mid and high affinity CD3 binding, respectively. CDR-12, CDR-13 and CDR-14 comprised Fab-(scFv)2 compounds with low, mid and high affinity CD3 binding, respectively. Low affinity CD3 binding lead to lower potency, while high and mid affinity CD3 binding showed increased cytotoxic effects, correlating with the increasing CD3 affinity.

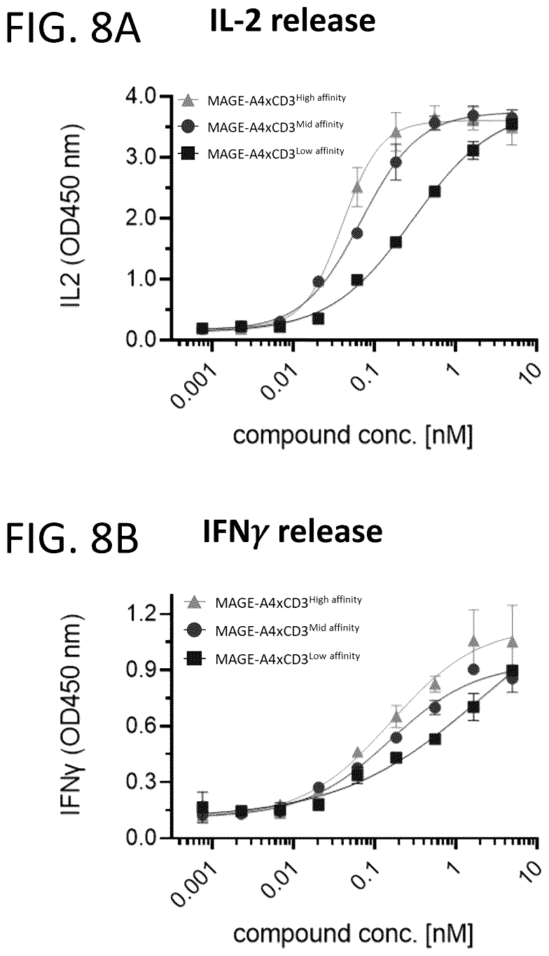

As shown in FIGS. 8A-8B, cytokine release was detected in antigen-positive osteosarcoma cells co-incubated with healthy donor PBMCs (E:T 10:1) and three dual pMHC-targeting T cell engagers in Fab-(VHH)2 format, each with a different level of binding affinity for CD3, i.e., low (CDR-9, 54 nM), mid (CDR-10, 11 nM) and high (CDR-11, 1.2 nM). Cytokines IL-2 and IFN gamma were measured at various concentrations of the three antigen binding proteins after a 24-hour incubation. The cytokines were measured using ELISA. Collectively, the results show that the level of cytokine release and potency can be tuned as necessary by changing the binding affinity of the anti-CD3 binding domain.

### Example 5—Potency of the Bivalent pMHC TCE is Strongly Influenced by the Intrinsic Affinity of the MAGE-A4 Binding Arms

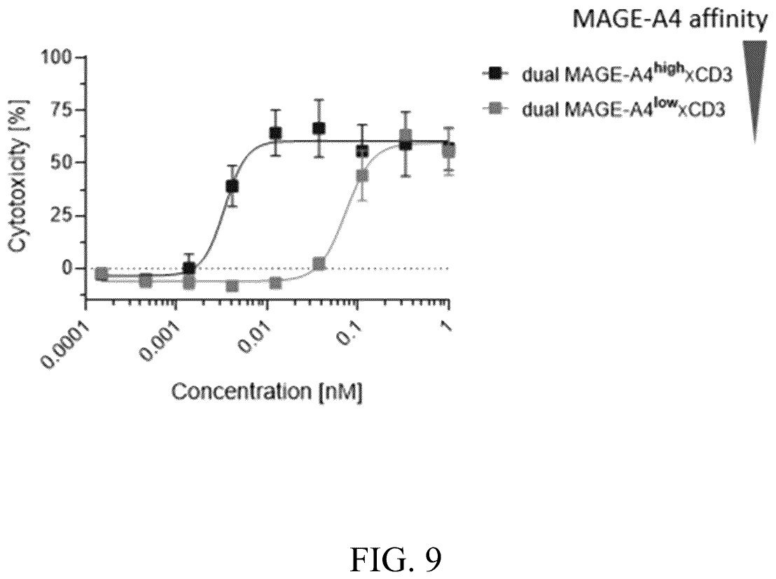

Dual pMHC-targeting T-cell engagers in Fab-(scFv)2 format comprising low (CDR-15) and high (CDR-8) affinity MAGE-A4 binders (KD of 41 nM and 0.1 nM, respectively) were evaluated for cell killing of MAGE-A4 positive U2OS cancer cells upon co-incubation with PBMCs (E:T 10:1). T cell-mediated cytotoxicity was determined by measuring LDH release after 48 h. The results as shown in FIG. 9 confirm that affinity enhancement of the MAGE-A4 binding arms mediates greater degree of cancer killing than the enhancement of the CD3 binding arm.

### Example 6—Dual pMHC TCE Shows High Selectivity Compared to sTCR Comparator

Dual pMHC TCE in Fab-(scFv)2 format (i.e., CDR-8) was analyzed for potential off-target effects by recognition of similar and physiologically relevant non-MAGE-A4 peptides. Applying in silico analysis of peptide sequence similarity combined with mass spectroscopy analysis of eluted HLA peptides, peptide databases and alanine scanning, the specificity of MAGE-A4/MHC-targeting antibodies was previously evaluated (KAMAR PELED et al., 2015). The identified similar peptides (S1, S16) with confirmed human tissue expression were separately loaded on the TAP-deficient T2 cells, which express empty HLA-A*02:01 molecules on the surface, for specificity assessment.

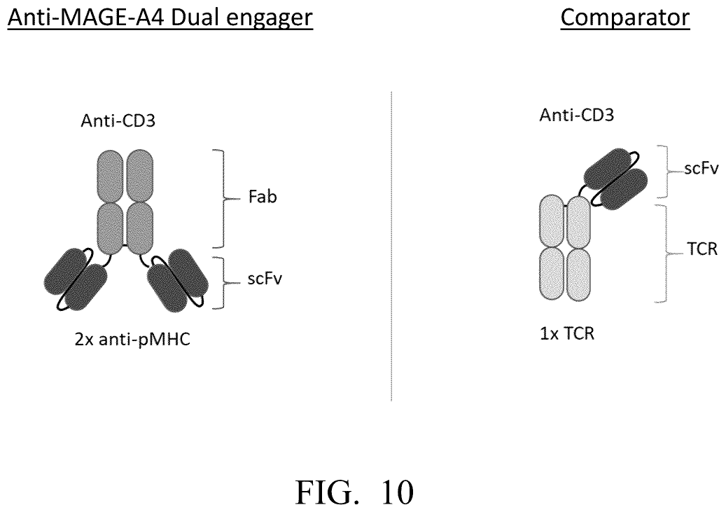

TAP-deficient T2 cells were pulsed with HLA-A*02:01-restricted peptides (MAGE-A4 or similar control peptides, deemed to be presented in relevant human tissues, S1 (GLADGRTHTV, SEQ ID NO.: 89) and S16 (GLYDGPVHEV, SEQ ID NO.: 90)) and co-incubated with PBMCs (E:T 5:1) and 0.1 nM of the dual pMHC-targeting TCE comprising the high affinity MAGE-A4 scFvs and mid affinity CD3 Fabs or an in-house produced clinical stage comparator molecule (sTCRxCD3). The Comparator is composed of a soluble TCR with binding specificity for the same pMHC-MAGE-A4 antigen with an 87 pM K_{D}, linked to an anti-CD3 scFv with a 1 nM K_{D} and therewith similar to the clinical stage IMC-C103C compound. The comparator is monovalent for the target pMHC and CD3, while the dual engager is bivalent for the target pMHC and monovalent for CD3. The comparator molecule and the dual engager are schematically depicted in FIG. 10.

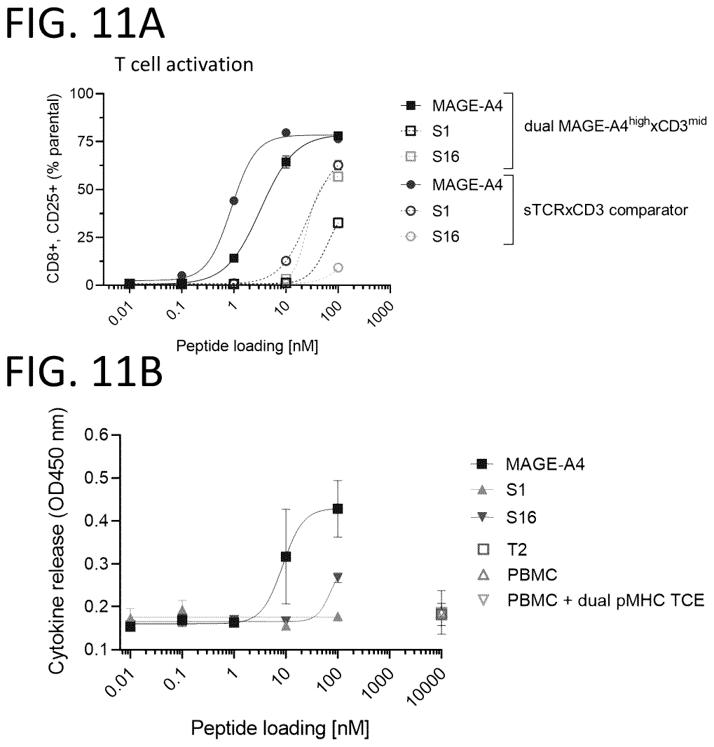

T cell activation was determined by quantification of CD25 markers on the CD8 T cell population after 24 h using flow cytometry, see FIG. 11A. T2 cells were treated as described above, incubated with 0.1 nM dual pMHC-targeting T cell engager comprising high MAGE-A4 and mid CD3 affinity. Cytokine release was determined by quantification of IFN-gamma in the cell supernatants after 24 h using ELISA (results depicted in FIG. 11B). The results show that the dual pMHC-targeting T cell engager (with picomolar affinity for MAGE-A4) elicits considerably lower T cell functional responses for the S1 and S16 off-target peptides than for the MAGE-A4 target peptide. Therefore, the bivalent targeting of MAGE-A4 does not compromise selectivity of the bispecific molecule since the T2 cells pulsed with similar physiologically relevant peptides and co-cultured with PBMC effector cells showed no significant T cell activation or IFNg release in the presence of the dual pMHC TCE in comparison to MAGE-A4 peptide-pulsed T2 cells.

### Example 7—Bivalent pMHC TCE Demonstrates Limited Cross-Reactivity Towards Antigen-Negative Cells In Vitro

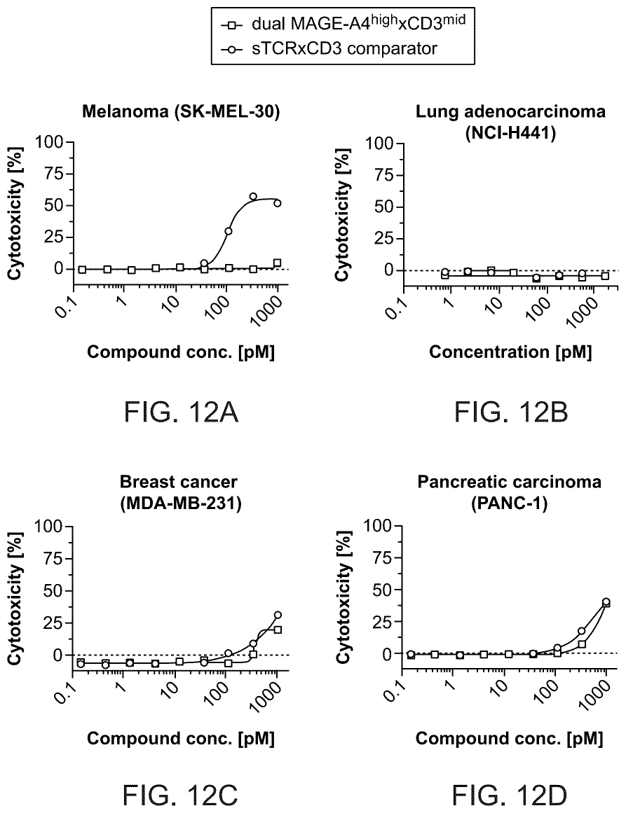

MAGE-A4 negative/HLA-A*02:01 positive cells (SK-MEL-30, NCI-H441, MDA-MB-231, PANC-1) were co-incubated with PBMCs (E:T 10:1) and either dual pMHC-targeting T cell engager in Fab-(scFv)2 format with picomolar MAGE-A4-targeting scFvs and a CD3-targeting Fab having mid CD3 affinity (i.e., CDR-8) or an in-house produced clinical stage comparator molecule as described in example 6. T cell-mediated cytotoxicity was determined by measuring LDH release after 48 h. Results are shown in FIGS. 12A-12D. Accordingly, dual pMHC-targeting T cell engager induces comparable or less cytotoxicity of MAGE-A4 negative/HLA-A*02:01 positive cells than sTCRxCD3 comparator.

### Example 8—Dual pMHC T Cell Engager Shows High Anti-Tumor Cytotoxicity Profile with Limited Cytokine Release

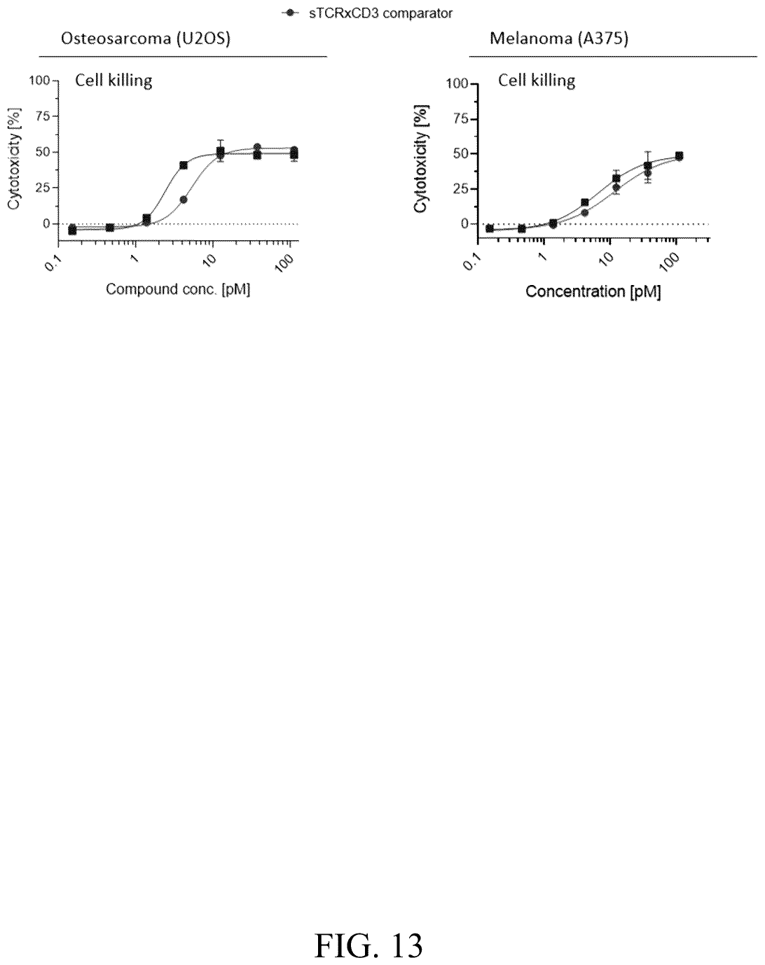

As shown in FIG. 13, percent cancer cell killing in osteosarcoma cells and melanoma cells incubated with the dual pMHC-targeting T cell engager in Fab-(scFv)2 format (i.e., CDR-8) or comparator as described in example 6 was measured. MAGE-A4 & HLA-A*02 positive cell lines A375 (melanoma) and U2OS (osteosarcoma) were incubated with human PBMCs at an E:T ratio of 10:1 for 48 h. Cancer cell killing was measured at various concentrations of the two antigen binding proteins. The data shows that the dual pMHC-targeting TCE more potently mediated killing of both cancer cell lines compared to Comparator. This was true even in the melanoma cell line, which only expresses a low copy number of the target pMHC-MAGE-A4 antigen (about 35 copies per cell).

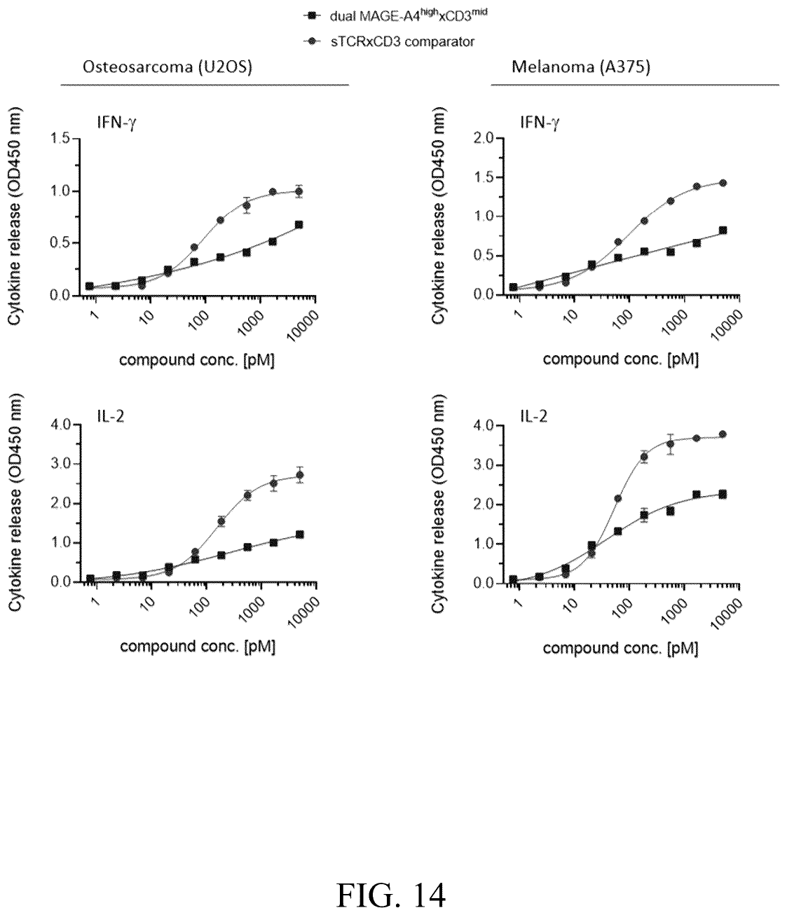

As shown in FIG. 14, cytokine release in osteosarcoma cells and melanoma cells incubated with the dual pMHC-targeting TCE (i.e., CDR-8) or Comparator was measured. Cytokine release was determined by quantification of IFN-gamma and IL-2 in the cell supernatants after 20 h using ELISA. MAGE-A4 & HLA-A*02 positive cell lines A375 (melanoma) and U2OS (osteosarcoma) were incubated with human PBMCs at an E:T ratio of 10:1. Cytokines IL-2 and IFN gamma were measured at various concentrations of the two antigen binding proteins. The data shows that the dual engager induced lower levels of the two pro-inflammatory cytokines, indicating a lower potential for inducing a cytokine storm syndrome.

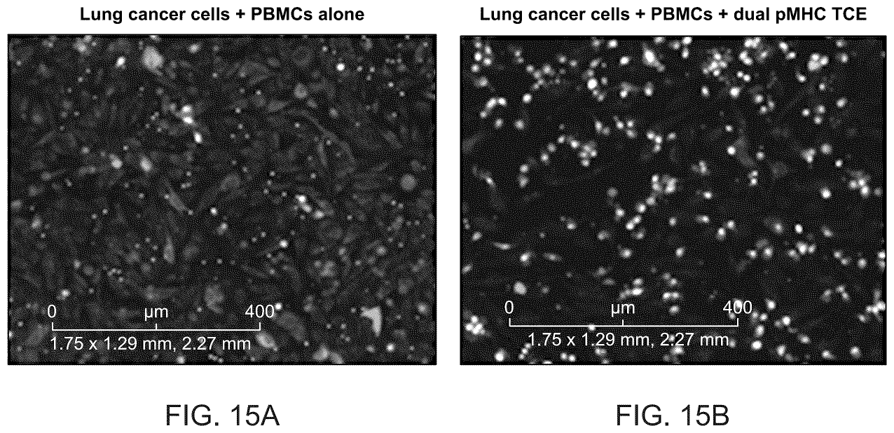

Live cell imaging of MAGE-A4 positive NCI-H1703 lung squamous carcinoma cells co-cultured with human PBMCs in presence of a dual pMHC TCE in Fab-(scFv)2 format (i.e., CDR-8) with specificity for MAGE-A4/HLA-A*02:01 was performed. Lung cancer cells were stained with Cytolight Rapid Red; cell death was revealed with Cytotox Green. As shown in FIGS. 15 A and B, the dual pMHC-targeting TCE elicits highly efficient anti-tumor responses.

### Example 9—Reduced Anti-Drug Antibodies (ADAs) with pMHC-Targeting T Cell Engagers

ADAs may affect the risk profile and efficacy of a biological drug. If neutralizing, they may block the drug's ability to bind to its target. It is therefore a regulatory requirement to test biologic drugs for the binding of anti-drug antibodies and their neutralizing potential. In addition, if the pre-existing Abs recognize the C-terminally located scFvs or sdAbs, clustering of T Cell engagers via binding to the pre-existing antibodies may occur. Such phenomenon could lead to generation of pre-existing ADA:bispecific Ab complexes with clustered free T cell engaging moieties. The presence of such complexes comprising multiple free CD3 binding arms could lead to avidity-driven T cell activation in the absence of cancer cells, which could in turn cause a cytokine release syndrome. The monovalent pMHC-targeting T cell engager was tested for its ability to evade ADA binding compared to sTCR comparator.

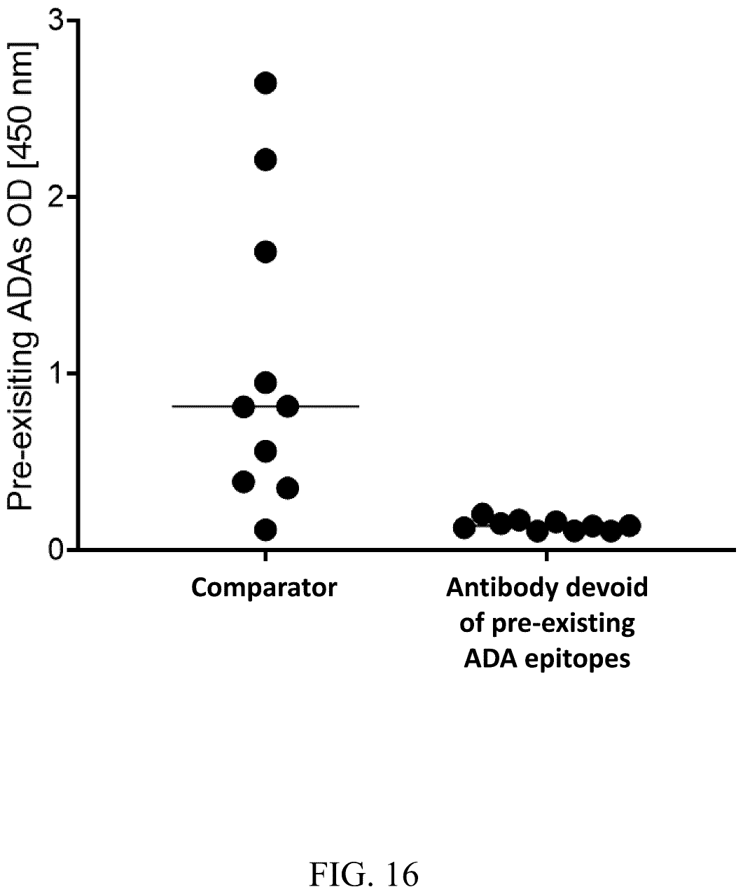

As shown in FIG. 16, pre-existing ADAs were quantified for the comparator, as described above, and the de-immunized sdAb compound CDR-16. Comparator and the de-immunized sdAb were evaluated with serum samples from 10 healthy naïve Caucasian human donors. Pre-existing ADAs were detected by ELISA. The data shows that the de-immunized sdAb was not targeted by ADAs, while Comparator was bound by ADAs.

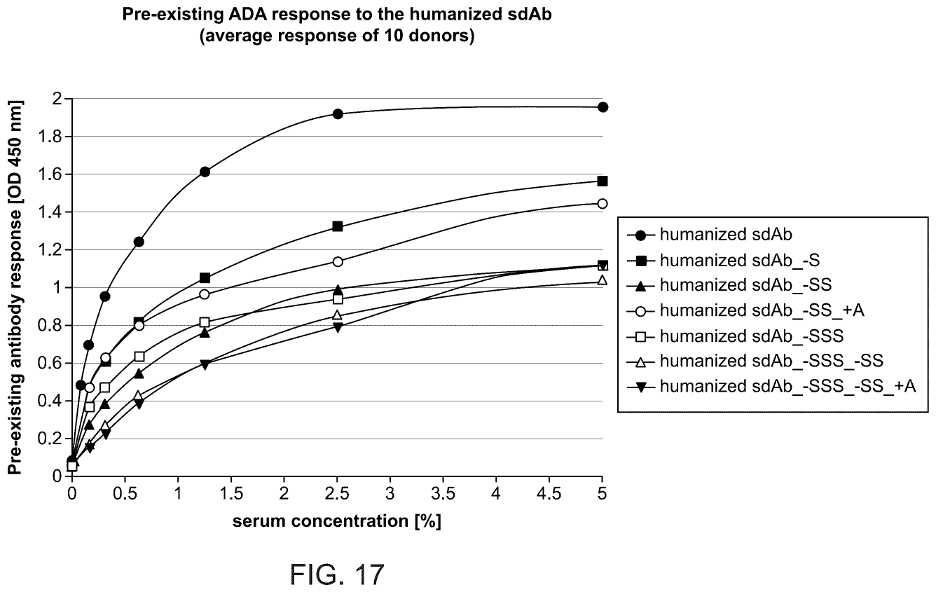

In an effort to reduce ADA engagement with the dual pMHC-targeting T cell engager format, amino acid modifications were generated in a single domain antibody format (sdAb) and an scFv format. As shown in FIG. 17, binding to pre-existing ADAs was quantified in humanized sdAb compound CDR-17 with selected modifications. “+A” corresponds to the addition of an alanine on C-terminus. “−S” corresponds to the deletion of a serine at position 113, according to Kabat numbering. “−SS” corresponds to the deletion of a serine at position 112 and 113, according to Kabat numbering. “SSS” corresponds to the substitution of hydrophobic amino acids at Kabat positions 11, 89, and 108 to serine amino acids. The triple serine substitution “SSS” is further described in WO2009/155725, incorporated herein by reference. The ADA response was measured with an ELISA over different sample serum concentrations. The data demonstrates that the inclusion of any one or more of the above modifications reduces binding to ADAs. The combination of SSS and −SS modifications or SSS, −SS, and A modifications reduced binding to ADA the most.

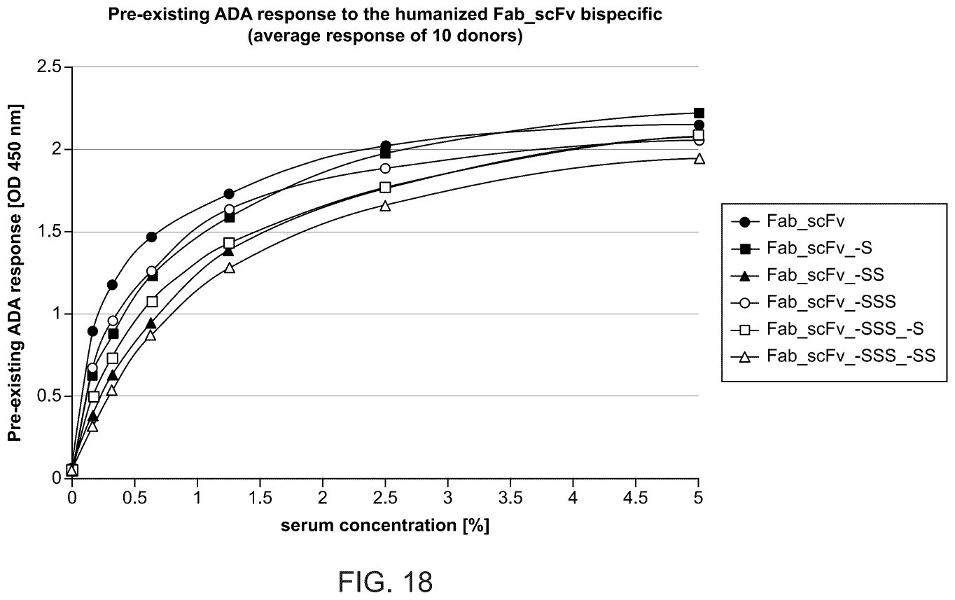

As shown in FIG. 18, binding to pre-existing ADAs was quantified for Fab-scFv antigen binding proteins based on compound CDR-18 with selected modifications on the scFv. “+A” corresponds to the addition of an alanine. “−S” corresponds to the deletion of a serine at position 113, according to Kabat numbering. “−SS” corresponds to the deletion of a serine at position 112 and 113, according to Kabat numbering. “SSS” corresponds to the substitution of hydrophobic amino acids at Kabat positions 11, 89, and 108 to serine amino acids. The ADA response was measured with an ELISA over different sample serum concentrations. The data demonstrates that the inclusion of any one or more of the above modifications reduces binding to ADAs.

1. An antigen binding protein comprising:
a) a single Fab domain which specifically binds to a cell surface protein of an immune cell, the Fab domain comprising a heavy chain and a light chain;
b) at least a first pMHC binding domain operably linked to the heavy chain, wherein the first pMHC binding domain binds to first target peptide-MHC (pMHC) complex; and
c) at least a second pMHC binding domain operably linked to the light chain, wherein the second pMHC binding domain binds to a second target pMHC complex,
wherein antigen binding protein does not comprise an Fc domain.

2. The antigen binding protein of claim 1, wherein:
the Fab domain heavy chain comprises a CHI domain and a VH domain, and 5 amino acids of an antibody hinge region, located at the C-terminus of the CHI domain of the Fab domain, or at least 5 amino acids of the antibody hinge, preferably 5-10 amino acids of the antibody hinge region;
the Fab domain light chain comprises a CL domain and a VL domain;
the first pMHC binding domain is operably linked to the C-terminus of the heavy chain or the N-terminus of the heavy chain;
the second pMHC binding domain is operably linked to the C-terminus of the light chain or the N-terminus of the light chain;
the first target pMHC complex and the second target pMHC complex are the same or are different; and/or
the first and/or second pMHC binding domain is a scFv, a sdAb, scFab, a diabody or a Fab, preferably a scFv or an sdAb.

3-8. (canceled)

9. The antigen binding protein of claim 1, comprising:
1) a first pMHC binding scFv linked to the C-terminus of the Fab domain heavy chain and a second pMHC binding scFv linked to the C-terminus of the Fab domain light chain;
2) a first pMHC binding scFv linked to the N-terminus of the Fab domain heavy chain and a second pMHC binding scFv linked to the N-terminus of the Fab domain light chain;
3) a first pMHC binding scFv linked to the N-terminus of the Fab domain heavy chain and a second pMHC binding scFv linked to the C-terminus of the Fab domain light chain;
4) a first pMHC binding scFv linked to the C-terminus of the Fab domain heavy chain and a second pMHC binding scFv linked to the N-terminus of the Fab domain light chain;
5) a first pMHC binding sdAb linked to the N-terminus of the Fab domain heavy chain and a second pMHC binding sdAb linked to the N-terminus of the Fab domain light chain;
6) a first pMHC binding sdAb linked to the C-terminus of the Fab domain heavy chain and a second pMHC binding sdAb linked to the C-terminus of the Fab domain light chain;
7) a first pMHC binding sdAb linked to the N-terminus of the Fab domain heavy chain and a second pMHC binding sdAb linked to the C-terminus of the Fab domain light chain; or
8) a first pMHC binding sdAb linked to the C-terminus of the Fab domain heavy chain and a second pMHC binding sdAb linked to the N-terminus of the Fab domain light chain.

10. The antigen binding protein of claim 1, wherein the Fab domain comprises a variable heavy chain having a non-polar amino acid at position 11, 89 and/or 108, according to Kabat numbering.

11. The antigen binding protein of claim 1, wherein the first pMHC binding domain and/or the second pMHC binding domain comprise a variable heavy chain having a polar amino acid at position 11, 89 and/or 108, according to Kabat numbering, optionally wherein the polar amino acid is serine (S) and/or threonine (T).

12. The antigen binding protein of claim 11, wherein the variable heavy chain comprises:
leucine (L) or serine (S) at amino acid position 11, according to Kabat numbering;
valine (V), serine (S), or threonine (T) at amino acid position 89, according to Kabat numbering; and/or
leucine (L), serine (S), or threonine (T) amino acid position 108, according to Kabat numbering.

13. (canceled)

14. The antigen binding protein of claim 11, wherein the variable heavy chain comprises serine (S) at amino acid position 11, serine (S) or threonine (T) at amino acid position 89, and serine (S) or threonine (T) at amino acid position 108, according to Kabat numbering.

15. The antigen binding protein of claim 11, wherein the variable heavy chain comprises serine (S) at amino acid position 11, serine (S) at amino acid position 89, and serine (S) at amino acid position 108, according to Kabat numbering.

16. The antigen binding protein of claim 1, wherein the first pMHC binding domain and/or the second pMHC binding domain comprises a variable heavy chain having a serine (S) at position 113 deleted, according to Kabat numbering.

17. (canceled)

18. The antigen binding protein of claim 1, wherein the first pMHC binding domain and/or the second pMHC binding domain comprises a variable heavy chain having a serine (S) at position 112 deleted and a serine (S) at position 113 deleted, according to Kabat numbering.

19. (canceled)

20. The antigen binding protein of claim 18, comprising an S113A, S113G, or S113T substitution, and optionally wherein S112 is deleted, according to Kabat numbering.

21. (canceled)

22. The antigen binding protein of claim 18, comprising an S112A, S112G, or S112T substitution, and optionally wherein S113 is deleted, according to Kabat numbering.

23. (canceled)

24. The antigen binding protein of claim 1, wherein the first and/or second target pMHC binding domain specifically targets an MHC restricted peptide derived of a tumor antigen or a viral antigen.

25. The antigen binding protein of claim 1, wherein the cell surface protein of an immune cell is selected from the group consisting of CD3, TCRα, TCRβ, CD16, NKG2D, CD89, CD64, and CD32a.

26. (canceled)

27. The antigen binding protein of claim 1, wherein the immune cell is selected from the group consisting of a T cell, a B cell, a natural killer (NK) cell, a natural killer T (NKT) cell, a neutrophil cell, a monocyte, and a macrophage.

28. (canceled)

29. The antigen binding protein of claim 1, wherein:
the Fab domain specifically binds to CD3 with a binding affinity (K_{D}) between about 1 nM to about 50 nM, as determined by SPR;
the Fab domain specifically binds to CD3 with an association rate constant kₐ of at least 1×10⁶ M⁻¹s⁻¹ or at least 2×10⁶ M⁻¹s⁻¹, as determined by SPR; and/or
the Fab domain specifically binds to CD3 with a dissociation rate constant k_{d} of at least 2×10⁻³ s⁻¹, or at least 3×10⁻³ s⁻¹ or at least 4×10⁻³ s⁻¹, as determined by SPR,
preferably wherein the association rate constant kₐ and/or the dissociation rate constant k_{d} are equivalent or similar for both CD3-heterodimers CD3εγ (epsilon/gamma) and CD3εδ (epsilon/delta).

30-35. (canceled)

36. The antigen binding protein of claim 1, wherein:
the first pMHC binding domain and/or the second pMHC binding domain binds the target pMHC complex with a binding affinity (K_{D}) of about 100 pM to about 20 nM, as determined by SPR;
the first pMHC binding domain and/or the second pMHC binding domain binds the target pMHC complex with an association rate constant kₐ of the pMHC binding domain is between about 0.5×10⁶ M⁻¹s⁻¹ to about 3×10⁶ M⁻¹s⁻¹, as determined by SPR, preferably wherein the association rate constant kₐ is as at least 0.5×10⁶ M⁻¹s⁻¹, at least 1×10⁶ M⁻¹s⁻¹, at least 2×10⁶ M⁻¹s⁻¹ or at least 3×10⁶ M⁻¹s⁻¹ as determined by SPR; and/or
the first pMHC binding domain and/or the second pMHC binding domain binds the target pMHC complex with a dissociation rate constant k_{d} of the pMHC binding domain is between about 1×10⁻² to about 1×10⁻⁵s⁻¹, as determined by SPR, preferably wherein the dissociation rate constant k_{d} is at least 2×10⁻³ s⁻¹, at least 4×10⁻³ s⁻¹, at least 6×10⁻³ s⁻¹, at least 8×10⁻³ s⁻¹, at least 2×10⁻⁴ s⁻¹ at least 4×10⁻⁴ s⁻¹, at least 6×10⁻⁴ s⁻¹ or at least 8×10⁻⁴ s⁻¹, as determined by SPR.

37-42. (canceled)

43. An antigen binding protein comprising:
a) a single Fab domain which specifically binds CD3 on a T cell, the Fab domain comprising a heavy chain and a light chain;
b) at least a first pMHC binding domain operably linked to the C-terminus of the heavy chain, wherein the first pMHC binding domain binds to first target peptide-MHC (pMHC) complex; and
c) at least a second pMHC binding domain operably linked to the C-terminus of the light chain, wherein the second pMHC binding domain binds to a second target pMHC complex, wherein antigen binding protein does not comprise an Fc domain.

44. A method for killing a target cell comprising a major histocompatibility complex (MHC) presenting a neoantigen, the method comprising:
a) contacting a plurality of cells comprising immune cells and the target cell with the antigen binding protein of claim 1, wherein said antigen binding protein specifically binds to the pMHC on the surface of the target cell and to CD3 on the surface of the immune cells;
b) forming a specific binding complex through the antigen binding protein interactions with the target cells and the immune cells, thereby activating the immune cells; and
c) killing the target cell with the activated immune cells.

45. (canceled)

46. A method of treating cancer comprising the step of administering the antigen binding protein of claim 1 to a patient in need thereof.

47. A nucleic acid encoding the antigen binding protein of claim 1.

48. An expression vector comprising the nucleic acid of claim 47.

49. A host cell population comprising the expression vector of claim 48.

50. A kit comprising the antigen binding protein of claim 1.

51. A method of manufacturing an antigen binding protein, comprising the steps of:
(i) cultivating the host cell of claim 49 under conditions allowing expression of the antigen binding protein;
(ii) recovering the antigen binding protein; and optionally
(iii) further purifying and/or modifying and/or formulating the antigen binding protein.

52. (canceled)

53. The antigen binding protein of claim 2, wherein said at least 5 amino acids of the antibody hinge is or comprises EPKSC, optionally followed by a GGGGS (SEQ ID NO.: 88) linker.

54. The antigen binding protein of claim 2, wherein said first pNMC binding domain is linked to the to the C-terminal end of the Fab CH1 domain via a linker selected from the group consisting of EPKSCGGGGS (SEQ ID NO.: 101), EPKSCDKTHT (SEQ ID NO.: 100), EPKSCDKTHTGGGGS (SEQ ID NO.: 102), DKTHT (SEQ ID NO.: 103), DKTHTGGGGS (SEQ ID NO.: 104) and GGGGSGGGGS (SEQ ID NO.: 105).

55. The antigen binding protein of claim 2, wherein said second pNMC binding domain is linked to the CL domain via a GGGGS (SEQ ID NO.:88) linker.

56. The antigen binding protein of claim 43, wherein the first pMHC binding domain is a scFv or a sdAb.

57. The antigen binding protein of claim 43, wherein the second pMHC binding domain is a scFv or a sdAb.

58. The antigen binding protein of claim 43, wherein the first and the second pMHC binding domains target the same pMHC complex, preferably bind to the same target epitope.

59. The antigen binding protein of claim 43, being a bispecific Fab-scFv2.

60. The antigen binding protein of claim 43, consisting of:
a) the single Fab domain;
b) the first pMHC binding domain; and
c) the second pMHC binding domain,
wherein the first pMHC binding domain and the second pMHC binding domain are identical.

61. The antigen binding protein of claim 43, consisting of:
a) the single Fab domain;
b) the first pMHC binding domain; and
c) the second pMHC binding domain,
wherein the first pMHC binding domain and the second pMHC binding domain are identical,
wherein said first pMHC binding domain is a scFv linked to the C-terminus of the Fab heavy chain via a EPKSCGGGGS (SEQ ID NO.: 101) linker, and
wherein said second pMHC binding domain is a scFv linked to the C-terminus of the Fab light chain via a GGGGS (SEQ ID NO.:88) linker.


SEQUENCE LISTING (106 sequences)

SEQ ID NO: 1 - protein, 10 aa, synthetic construct
GVYDGREHTV

SEQ ID NO: 2 - protein, 6 aa, synthetic construct
SNYAMS

SEQ ID NO: 3 - protein, 16 aa, synthetic construct
IVSSGGTTYYADSVKG

SEQ ID NO: 4 - protein, 15 aa, synthetic construct
DLYYGPNTDYSAANL

SEQ ID NO: 5 - protein, 11 aa, synthetic construct
TADTLSRSYAS

SEQ ID NO: 6 - protein, 7 aa, synthetic construct
RDTSRPS

SEQ ID NO: 7 - protein, 12 aa, synthetic construct
ATRPSSGSNFQA

SEQ ID NO: 8 - protein, 6 aa, synthetic construct
SNYAMS

SEQ ID NO: 9 - protein, 16 aa, synthetic construct
IVSSGGTTYYADSVKG

SEQ ID NO: 10 - protein, 14 aa, synthetic construct
DLYYGPSTYFVANL

SEQ ID NO: 11 - protein, 11 aa, synthetic construct
TADTLSRSYAS

SEQ ID NO: 12 - protein, 7 aa, synthetic construct
RDTSRPS

SEQ ID NO: 13 - protein, 12 aa, synthetic construct
ATRPSSGSNFQL

SEQ ID NO: 14 - protein, 6 aa, unidentified
SNYAMS

SEQ ID NO: 15 - protein, 16 aa, synthetic construct
IVSSGGTTYYASWAKG

SEQ ID NO: 16 - protein, 14 aa, synthetic construct
DLYYGPTTYSAANL

SEQ ID NO: 17 - protein, 11 aa, synthetic construct
TADTLSRSYAS

SEQ ID NO: 18 - protein, 7 aa, synthetic construct
RDTSRPS

SEQ ID NO: 19 - protein, 12 aa, synthetic construct
ATRDFSGSNFQL

SEQ ID NO: 20 - protein, 6 aa, synthetic construct
SNYAMS

SEQ ID NO: 21 - protein, 16 aa, synthetic construct
IVSSGGTTYYASWAKG

SEQ ID NO: 22 - protein, 14 aa, synthetic construct
DLYYGPTTYSAFNL

SEQ ID NO: 23 - protein, 11 aa, synthetic construct
TADTLSRSYAS

SEQ ID NO: 24 - protein, 7 aa, synthetic construct
RDTSRPS

SEQ ID NO: 25 - protein, 12 aa, synthetic construct
ATRPSSGSNFQA

SEQ ID NO: 26 - protein, 6 aa, synthetic construct
SNYAMS

SEQ ID NO: 27 - protein, 16 aa, synthetic construct
  VAR_SEQ 12: note = corresponds to amino acid S or D
  VAR_SEQ 13: note = corresponds to amino acid W or S
  VAR_SEQ 14: note = corresponds to amino acid A or V
IVSSGGTTYYAXXXKG

SEQ ID NO: 28 - protein, 15 aa, synthetic construct
  VAR_SEQ 7: note = corresponds to amino acid T, N, or S
  VAR_SEQ 9: note = corresponds to amino acid D or is absent
  VAR_SEQ 11: note = corresponds to amino acid S or F
  VAR_SEQ 12: note = corresponds to amino acid A or V
  VAR_SEQ 13: note = corresponds to amino acid F or A
DLYYGPXTXYXXXNL

SEQ ID NO: 29 - protein, 11 aa, synthetic construct
TADTLSRSYAS

SEQ ID NO: 30 - protein, 7 aa, synthetic construct
RDTSRPS

SEQ ID NO: 31 - protein, 12 aa, synthetic construct
  VAR_SEQ 3: note = corresponds to amino acid S or R
  VAR_SEQ 4: note = corresponds to amino acid D or P
  VAR_SEQ 5: note = corresponds to amino acid G, S, or F
  VAR_SEQ 12: note = corresponds to amino acid L or A
ATXXXSGSNFQX

SEQ ID NO: 32 - protein, 123 aa, synthetic construct
EVQLLESGGGSVQPGGSLRLSCTVSGFSLSNYAMSWVRQAPGKGLEWIGIVSSGGTTYYADSVKGRFTISRDNSKNTVYLQMNSLRAEDTASYYCAKDLYYGPNTDYSAANLWGQGTSVTVSS

SEQ ID NO: 33 - protein, 110 aa, synthetic construct
QSVLTQDPAVSVALGQTVRITCTADTLSRSYASWYQQKPGQAPVLVIYRDTSRPSGIPDRFSGSSSGNTASLTITGAQAEDEADYYCATRPSSGSNFQAFGGGTKLTVLG

SEQ ID NO: 34 - protein, 122 aa, synthetic construct
EVQLLESGGGSVQPGGSLRLSCTVSGFSLSNYAMSWVRQAPGKCLEWIGIVSSGGTTYYADSVKGRFTISRDNSKNTVYLQMNSLRAEDTASYYCAKDLYYGPSTYFVANLWGQGTSVTVSS

SEQ ID NO: 35 - protein, 110 aa, synthetic construct
QSVLTQDPAVSVALGQTVRITCTADTLSRSYASWYQQKPGQAPVLVIYRDTSRPSGIPDRFSGSSSGNTASLTITGAQAEDEADYYCATRPSSGSNFQLFGCGTKLTVLG

SEQ ID NO: 36 - protein, 122 aa, synthetic construct
EVQLLESGGGSVQPGGSLRLSCTVSGFSLSNYAMSWVRQAPGKCLEWIGIVSSGGTTYYASWAKGRFTISKDTSKNTVYLQMNSLRAEDTASYYCAKDLYYGPTTYSAANLWGQGTSVTVSS

SEQ ID NO: 37 - protein, 110 aa, synthetic construct
SYELTQPPSVSVSPGQTASITCTADTLSRSYASWYQQKPGQSPVLVIYRDTSRPSGIPERFSGSNSGNTATLTISGTQAMDEADYYCATRDFSGSNFQLFGCGTKLTVLG

SEQ ID NO: 38 - protein, 122 aa, synthetic construct
EVQLLESGGGSVQPGGSLRLSCTVSGFSLSNYAMSWVRQAPGKGLEYIGIVSSGGTTYYASWAKGRFTISRDNSKNTVYLQMNSLRAEDTASYYCAKDLYYGPTTYSAFNLWGQGTSVTVSS

SEQ ID NO: 39 - protein, 110 aa, synthetic construct
SYELTQPPSVSVSPGQTASITCTADTLSRSYASWYQQKPGQSPVLVIYRDTSRPSGIPERFSGSNSGNTATLTISGTQAMDEADYYCATRPSSGSNFQAFGGGTKLTVLG

SEQ ID NO: 40 - protein, 228 aa, synthetic construct
EVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 41 - protein, 348 aa, synthetic construct
EVQLVESGGGLAQAGGSLRVSCVASGRPFTKYAWGWFRQAPGKAREFVATITWDGGKTDYADSVKGRFTISKDSAENSIYLQMNSLKPEDTAVYYCAADRNYCVGHRCYVRPDDYDYWGQGTQVTVSSGGGGSAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 228 aa, synthetic construct; identical to SEQ ID NO: 40

SEQ ID NO: 43 - protein, 348 aa, synthetic construct
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLAQAGGSLRVSCVASGRPFTKYAWGWFRQAPGKAREFVATITWDGGKTDYADSVKGRFTISKDSAENSIYLQMNSLKPEDTAVYYCAADRNYCVGHRCYVRPDDYDYWGQGTQVTVSS

SEQ ID NO: 44 - protein, 361 aa, synthetic construct
EVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEVQLVESGGGLAQAGGSLRVSCVASGRPFTKYAWGWFRQAPGKAREFVATITWDGGKTDYADSVKGRFTISKDSAENSIYLQMNSLKPEDTAVYYCAADRNYCVGHRCYVRPDDYDYWGQGTQVTVSS

SEQ ID NO: 45 - protein, 215 aa, synthetic construct
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 361 aa, synthetic construct
EVQLVESGGGLAQAGGSLRVSCVASGRPFTKYAWGWFRQAPGKAREFVATITWDGGKTDYADSVKGRFTISKDSAENSIYLQMNSLKPEDTAVYYCAADRNYCVGHRCYVRPDDYDYWGQGTQVTVSSGGGGSEVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 47 - protein, 215 aa, synthetic construct; identical to SEQ ID NO: 45

SEQ ID NO: 48 - protein, 361 aa, synthetic construct; identical to SEQ ID NO: 44

SEQ ID NO: 49 - protein, 348 aa, synthetic construct; identical to SEQ ID NO: 41

SEQ ID NO: 50 - protein, 361 aa, synthetic construct; identical to SEQ ID NO: 44

SEQ ID NO: 51 - protein, 348 aa, synthetic construct; identical to SEQ ID NO: 43

SEQ ID NO: 52 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSYELTQPPSVSVSPGQTASITCTADTLSRSYASWYQQKPGQSPVLVIYRDTSRPSGIPERFSGSNSGNTATLTISGTQAMDEADYYCATRPSSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 53 - protein, 215 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - protein, 486 aa, synthetic construct; identical to SEQ ID NO: 52

SEQ ID NO: 55 - protein, 473 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSSYELTQPPSVSVSPGQTASITCTADTLSRSYASWYQQKPGQSPVLVIYRDTSRPSGIPERFSGSNSGNTATLTISGTQAMDEADYYCATRPSSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 56 - protein, 361 aa, synthetic construct
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 57 - protein, 348 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVS

SEQ ID NO: 58 - protein, 361 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 59 - protein, 349 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 60 - protein, 361 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 61 - protein, 349 aa, synthetic construct
QAVVTQEPSLTVSPGGTVTLTCRSSTGAVTTSNYANWVQQKPGQAPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 62 - protein, 486 aa, synthetic construct
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 63 - protein, 474 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 64 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 65 - protein, 474 aa, synthetic construct; identical to SEQ ID NO: 63

SEQ ID NO: 66 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 67 - protein, 474 aa, synthetic construct
QAVVTQEPSLTVSPGGTVTLTCRSSTGAVTTSNYANWVQQKPGQAPRGLIGGTNKRAPGVPARFSGSLLGGKAALTISGAQPEDEADYYCALWYSNHWVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTYSAFNLWGQGTSVTVSS

SEQ ID NO: 68 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTQSAFNLWGQGTSVTVSS

SEQ ID NO: 69 - protein, 473 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSSYELTQPPSVSVSPGQTASITCTADTLSRSYASWYQQKPGQSPVLVIYRDTSRPSGIPERFSGSNSGNTATLTISGTQAMDEADYYCATSDGSGSNFQLFGGGTKLTVLGGGGGGSGGGGSGGGGSGGGGSESQVLESGGGSVQPGGSLRLSCTVSGFSLSNYAMSWVRQAPGKGLEYIGIVSSGGTTYYASWAKGRFTISKDTSKNTVYLQMNSLRAEDTASYYCAKDLYYGPTTQSAFNLWGQGTSVTVSS

SEQ ID NO: 70 - protein, 200 aa, synthetic construct
MANQVEQSPQSLIILEGKNVTLQCNYTVSPFSNLRWYKQDTGRGPVSLTILDYAINTKSNGRYTATLDADTKQSSLHITASQLSDSASYICVVNRADGLYIPTFGRGTSLIVHPYIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 71 - protein, 506 aa, synthetic construct
MAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDVKVTQSSRYLVKRTGEKVFLECVQDAPLSKMFWYRQDPGLGLRLIYFSYDVKLKEKGDIPEGYSVSREKKERFSLILESASTNQTSMYLCASSSDQNSGDPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 72 - protein, 129 aa, synthetic construct
EVQLVESGGGSAQAGGSLRVSCVASGRPFTKYAWGWFRQAPGKAREFVATITWDGGKTDYADSVKGRFTISKDSAENSIYLQMNSLKPEDTASYYCAADRNYCVGHRCYVRPDDYDYWGQGTSVTVSSA

SEQ ID NO: 73 - protein, 128 aa, synthetic construct
EVQLVESGGGLVQPGGSLRLSCVASGRPFTKYAWGWFRQAPGKAREFVATITWDGGKTDYADSVKGRFTISKDSAKNSIYLQMNSLRAEDTAVYYCAADRNYCVGHRCYVRPDDYDYWGQGTLVTVSS

SEQ ID NO: 74 - protein, 223 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 75 - protein, 474 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSQAVVTQEPSLTVSPGGTVTLTCRSSTGAVTTSNYANWVQQKPGQAPRGLIGGTNKRAPGVPARFSGSLLGGKAALTISGAQPEDEADYYCALWYSNHWVFGGGTKLTVLGGGGGSGGGGSGGGGSGGGGSEVQLVESGGGSVQPGGSLRLSCAASGFTFSTYAMNWVRQAPGKGLEWVGRIRSKYNNYATYYADSVKGRFTISRDDSKNTLYLQMNSLRAEDTAMYYCVRHGNFGNSYVSWFAYWGQGTTVTVSS

SEQ ID NO: 76 - protein, 6 aa, synthetic construct
STYAMN

SEQ ID NO: 77 - protein, 19 aa, synthetic construct
RIRSKANNYATYYADSVKG

SEQ ID NO: 78 - protein, 14 aa, synthetic construct
HGNFGDSYVSWFAY

SEQ ID NO: 79 - protein, 14 aa, synthetic construct
GSSTGAVTTSNYAN

SEQ ID NO: 80 - protein, 14 aa, synthetic construct
RSSTGAVTTSNYAN

SEQ ID NO: 81 - protein, 7 aa, synthetic construct
GTNKRAP

SEQ ID NO: 82 - protein, 9 aa, synthetic construct
ALWYSNHWV

SEQ ID NO: 83 - protein, 109 aa, synthetic construct
AVVTQEPSLTVSPGGTVTLTCRSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 84 - protein, 125 aa, synthetic construct
EVQLVESGGGSVQPGGSLRLSCAASGFTFSTYAMNWVRQAPGKGLEWVGRIRSKFNNYATYYADSVKGRFTISRDDSKNTLYLQMNSLRAEDTATYYCVRHGNFGDSYVSWFAYWGQGTTVTVSS

SEQ ID NO: 85 - protein, 108 aa, synthetic construct
AVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 86 - protein, 125 aa, synthetic construct
EVQLVESGGGSVQPGGSLRLSCAASGFTFSTYAMNWVRQAPGKGLEWVGRIRSKANNYATYYADSVKGRFTISRDDSKNTLYLQMNSLRAEDTATYYCVRHGNFGDSYVSWFAYWGQGTTVTVSS

SEQ ID NO: 87 - protein, 5 aa, synthetic construct
EPKSC

SEQ ID NO: 88 - protein, 5 aa, synthetic construct
GGGGS

SEQ ID NO: 89 - protein, 10 aa, synthetic construct
GLADGRTHTV

SEQ ID NO: 90 - protein, 10 aa, synthetic construct
GLYDGPVHEV

SEQ ID NO: 91 - protein, 5 aa, synthetic construct
DGGGS

SEQ ID NO: 92 - protein, 5 aa, synthetic construct
TGEKP

SEQ ID NO: 93 - protein, 4 aa, synthetic construct
GGRR

SEQ ID NO: 94 - protein, 14 aa, synthetic construct
EGKSSGSGSESKVD

SEQ ID NO: 95 - protein, 18 aa, synthetic construct
KESGSVSSEQLAQFRSLD

SEQ ID NO: 96 - protein, 8 aa, synthetic construct
GGRRGGGS

SEQ ID NO: 97 - protein, 9 aa, synthetic construct
LRQRDGERP

SEQ ID NO: 98 - protein, 12 aa, synthetic construct
LRQKDGGGSERP

SEQ ID NO: 99 - protein, 18 aa, synthetic construct
GSTSGSGKPGSGEGSTKG

SEQ ID NO: 100 - protein, 10 aa, synthetic construct
EPKSCDKTHT

SEQ ID NO: 101 - protein, 10 aa, synthetic construct
EPKSCGGGGS

SEQ ID NO: 102 - protein, 15 aa, synthetic construct
EPKSCDKTHTGGGGS

SEQ ID NO: 103 - protein, 5 aa, synthetic construct
DKTHT

SEQ ID NO: 104 - protein, 10 aa, synthetic construct
DKTHTGGGGS

SEQ ID NO: 105 - protein, 10 aa, synthetic construct
GGGGSGGGGS

SEQ ID NO: 106 - protein, 20 aa, synthetic construct
GGGGSGGGGSGGGGSGGGGS